(19) United States
(12) Patent Application Publication
(10) Pub. No.: US 2023/0348995 A1
(43) Pub. Date: Nov. 2, 2023

(54) DIAGNOSTIC AND THERAPEUTIC METHODS FOR LYMPHOMA

(71) Applicant: Genentech, Inc., South San Francisco, CA (US)

(72) Inventors: Joseph Nathaniel PAULSON, San Francisco, CA (US); Aikaterini CHATZI, San Francisco, CA (US)

(21) Appl. No.: 18/163,819

(22) Filed: Feb. 2, 2023

Related U.S. Application Data
(63) Continuation of application No. PCT/US21/44404, filed on Aug. 3, 2021.
(60) Provisional application No. 63/060,598, filed on Aug. 3, 2020.

(51) Int. Cl. C12Q 1/6886; C07K 16/28

(52) U.S. Cl. CPC C12Q 1/6886; C07K 16/2887; C12Q 2600/106; C12Q 2600/158

(57) ABSTRACT

The present invention provides diagnostic methods, therapeutic methods, and compositions for the treatment of lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). The invention is based, at least in part, on the discovery that macrophage biomarkers and Th2 biomarkers are useful in methods of identifying, diagnosing, or predicting the therapeutic efficacy of treatment with an anti-CD20 antibody (e.g., obinutuzumab or rituximab).

### SEQUENCE LISTING

The instant application contains a Sequence Listing which has been submitted electronically in XML format and is hereby incorporated by reference in its entirety. Said XML copy, created on Jan. 26, 2023 is named 50474_212003_Sequence_Listing_01_26_23.xml and is 26,274 bytes in size.

### FIELD OF THE INVENTION

The present invention is directed to diagnostic and therapeutic methods for the treatment of lymphoma using an anti-CD20 antibody. Also provided are related assays and kits.

### BACKGROUND OF THE INVENTION

In the U.S., lymphoma is projected to cause approximately 20,000 deaths and account for approximately 85,000 new cancer cases in 2020. Although there have been significant advances in the treatment of lymphoma, the 5-year survival rate is only approximately 73%.

Diffuse large B-cell lymphoma (DLBCL) is the most common type of aggressive non-Hodgkin lymphoma (NHL). Immunochemotherapy with a combination of the type I anti-CD20 monoclonal antibody, rituximab, plus cyclophosphamide, doxorubicin, vincristine, and prednisone (CHOP) is standard-of-care treatment for previously untreated patients who present with advanced-stage disease. Although first-line treatment for DLBCL is potentially curative, many patients do not respond or eventually relapse.

Therefore, there is an unmet need for improved diagnostic and therapeutic methods for the treatment of DLBCL patients.

### SUMMARY OF THE INVENTION

The present invention relates to using macrophage biomarkers or Th2 biomarkers in methods of identifying, diagnosing, or predicting the therapeutic efficacy of treating lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with diagnostic methods, therapeutic methods, and compositions for the treatment of an anti-CD20 antibody (e.g., a type II (e.g., obinutuzumab) or a type I (e.g., rituximab) anti-CD20 antibody).

In a first aspect, the invention features a method of identifying, diagnosing, and/or predicting whether a patient having a lymphoma may benefit from a treatment comprising an anti-CD20 antibody, the method comprising measuring a macrophage biomarker in a sample from the patient, wherein an amount or level of the macrophage biomarker in the sample that is above a reference macrophage biomarker amount or level identifies, diagnoses, and/or predicts the patient as one who may benefit from a treatment comprising an anti-CD20 antibody.

In a second aspect, the invention features a method of selecting a therapy for a patient having a lymphoma, the method comprising measuring a macrophage biomarker in a sample from the patient, wherein an amount or level of the macrophage biomarker in the sample that is above a reference macrophage biomarker amount or level identifies the patient as one who may benefit from a treatment comprising an anti-CD20 antibody.

In some embodiments of the first or second aspects, the patient has a macrophage biomarker in the sample that is above a reference macrophage biomarker amount or level, and the method further comprises administering to the patient an effective amount of an anti-CD20 antibody.

In a third aspect, the invention features a method of treating a patient having a lymphoma, the method comprising: (a) measuring a macrophage biomarker in a sample from the patient, wherein the amount or level of the macrophage biomarker in the sample is above a reference macrophage biomarker amount or level, and (b) administering an effective amount of an anti-CD20 antibody to the patient based on the macrophage biomarker measured in step (a).

In a fourth aspect, the invention features a method of treating a patient having a lymphoma, the method comprising administering to the patient an effective amount of an anti-CD20 antibody, wherein prior to treatment the amount or level of a macrophage biomarker in a sample from the patient has been determined to be above a reference macrophage biomarker amount or level.

In a fifth aspect, the invention features a method of treating a patient having a lymphoma and having an amount or level of a macrophage biomarker in a sample from the patient that is above a reference macrophage biomarker amount or level comprising administering to the patient an effective amount of an anti-CD20 antibody.

In some embodiments of any of the first, second, third, fourth, and fifth aspects, the reference macrophage biomarker amount or level is a pre-assigned macrophage biomarker amount or level.

In some embodiments of any of the first, second, third, fourth, and fifth aspects, the reference macrophage biomarker amount or level is an amount or level of a macrophage biomarker in a reference population. In some embodiments, the amount or level of the macrophage biomarker in a reference population is a median amount or level of the macrophage biomarker of the reference population. In some embodiments, the reference macrophage biomarker amount or level is an amount or level of a macrophage biomarker that is at the 25th percentile of the reference population. In some embodiments, the reference macrophage biomarker amount or level is an amount or level of a macrophage biomarker that is at the 50th percentile of the reference population. In some embodiments, the reference macrophage biomarker amount or level is an amount or level of a macrophage biomarker that is at the 75th percentile of the reference population. In some embodiments, the reference population is a population of patients having the lymphoma. In some embodiments, the population of patients having the lymphoma was previously treated with an anti-CD20 antibody. In some embodiments, the reference macrophage biomarker amount or level is the amount or level of macrophage biomarker of the reference population prior to initiating treatment with the anti-CD20 antibody. In some embodiments, the reference macrophage biomarker amount or level significantly separates the reference population into a first set of patients who have benefitted from the treatment with the anti-CD20 antibody and a second set of patients who have not benefitted from the treatment with the anti-CD20 antibody.

In some embodiments of any of the first, second, third, fourth, and fifth aspects, the reference macrophage biomarker amount or level is an amount of macrophages as measured by gene expression. In some embodiments, the amount of macrophages is between about 0% to about 30.7%.

In some embodiments of the first or second aspects, the benefit is an extension of progression-free survival (PFS).

In some embodiments of the first or second aspects, the benefit is an increase in overall survival (OS).

In some embodiments of any of the third, fourth, and fifth aspects, the method further comprises achieving an improvement of PFS or OS.

In some embodiments of any of the first, second, third, fourth, and fifth aspects, the macrophage biomarker is an average of M1 macrophage gene signature set scores of one or more M1 macrophage gene signature sets. In some embodiments, each M1 macrophage gene signature set score is an average of the expression level of one or more genes of an M1 macrophage gene signature set. In some embodiments, each M1 macrophage gene signature set score is an average of the normalized expression level of one or more genes of an M1 macrophage gene signature set. In some embodiments, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB31L1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some embodiments of any of the first, second, third, fourth, and fifth aspects, the macrophage biomarker is an average of macrophage gene signature set scores of one or more macrophage gene signature sets. In some embodiments, each macrophage gene signature set score is an average of the expression level of one or more genes of a macrophage gene signature set. In some embodiments, each macrophage gene signature set score is an average of the normalized expression level of one or more genes of a macrophage gene signature set. In some embodiments, the one or more macrophage gene signature sets are any of the macrophage gene signature sets in Table 2.

In some embodiments of any of the first, second, third, fourth, and fifth aspects, the macrophage biomarker is a gene expression value. In some embodiments, the gene expression value is a median gene expression value. In some embodiments, the gene expression value is measured using a gene signature matrix. In some embodiments, the gene signature matrix comprises the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1. In some embodiments, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some embodiments, the gene signature matrix is used to determine a number of macrophages. In some embodiments, the gene signature matrix is used to determine a number of M1 macrophages.

In some embodiments of any of the first, second, third, fourth, and fifth aspects, the macrophage biomarker is an amount of M1 macrophages. In some embodiments, the amount of M1 macrophages is measured directly or indirectly. In some embodiments, the amount of M1 macrophages is measured directly using flow cytometry, spatial transcriptomics, spatial proteomics, or combination thereof. In some embodiments, the amount of M1 macrophages is measured indirectly using nucleic acid or protein. In some embodiments, the nucleic acid is measured using RNA-seq, RT-qPCR, qPCR, multiplex qPCR or RT-qPCR, microarray analysis, SAGE, MassARRAY technique, ISH, or a combination thereof. In some embodiments, the amount of M1 macrophages is measured using a marker gene approach or a deconvolution approach. In some embodiments, the marker gene approach uses xCell. In some embodiments, the deconvolution approach uses quanTlseq.

In some embodiments of any of the first, second, third, fourth, and fifth aspects, the macrophage biomarker in the sample from the patient is measured using nucleic acid or protein. In some embodiments, the macrophage biomarker in the sample from the patient is determined using a nucleic acid expression level. In some embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some embodiments, the nucleic acid expression level is an mRNA expression level. In some embodiments, the mRNA expression level is determined by RNA-seq.

In some embodiments of any of the first, second, third, fourth, and fifth aspects, the sample is a tissue sample, tumor sample, whole blood sample, a plasma sample, a serum sample, or a combination thereof. In some embodiments, the sample is a tissue sample. In some embodiments, the tissue sample is a tumor tissue sample. In some embodiments, the tumor tissue sample contains tumor cells, tumor-infiltrating immune cells, stromal cells, normal adjacent tissue (NAT) cells, or a combination thereof. In some embodiments, the tumor tissue sample is a biopsy. In some embodiments, the sample is an archival sample, a fresh sample, or a frozen sample.

In some embodiments of any of the first, second, third, fourth, and fifth aspects, the lymphoma is an indolent lymphoma.

In some embodiments of any of the first, second, third, fourth, and fifth aspects, the lymphoma is a B-cell lymphoma. In some embodiments, the B-cell lymphoma is a germinal center derived B-cell lymphoma. In some embodiments, the B-cell lymphoma is a non-Hodgkin lymphoma (NHL). In some embodiments of any of the first, second, third, fourth, and fifth aspects, the lymphoma is a diffuse large B-cell lymphoma (DLBCL), a follicular lymphoma (FL), a chronic lymphocytic leukemia (CLL), or a marginal zone lymphoma (MZL). In some embodiments, the lymphoma is a DLBCL. In some embodiments, the DLBCL is a germinal-center B-cell-like (GCB) or activated B-cell-like (ABC) cell-of-origin subgroup of DLBCL. In some embodiments of any of the first, second, third, fourth, and fifth aspects, the lymphoma is a CD20-positive lymphoma. In some embodiments of any of the first, second, third, fourth, and fifth aspects, the anti-CD20 antibody is a type I anti-CD20 antibody or a type II anti-CD20 antibody. In some embodiments, the anti-CD20 antibody is a type II anti-CD20 antibody. In some embodiments, the type II anti-CD20 antibody comprises the following complementarity determining regions (CDRs): (a) a CDR-H1 with an amino acid sequence of SEQ ID NO: 1; (b) a CDR-H2 with an amino acid sequence of SEQ ID NO: 2; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises a VH domain comprising an amino acid sequence of SEQ ID NO: 7 and a VL domain comprising an amino acid sequence of SEQ ID NO: 8. In some embodiments, the type II anti-CD20 antibody comprises the following CDRs: (a) a CDR-H1 with an amino acid sequence of SEQ ID NO: 27; (b) a CDR-H2 with an amino acid sequence of SEQ ID NO: 28; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises a VH domain comprising an amino acid sequence of SEQ ID NO: 7 and a VL domain comprising an amino acid sequence of SEQ ID NO: 8. In some embodiments, the type II anti-CD20 antibody is obinutuzumab. In some embodiments, the anti-CD20 antibody is a type I anti-CD20 antibody. In some embodiments, the type I anti-CD20 antibody comprises the following CDRs: (a) a CDR-H1 with an amino acid sequence of SEQ ID NO: 11; (b) a CDR-H2 with an amino acid sequence of SEQ ID NO: 12; (c) a CDR-H3 with an amino acid sequence of SEQ ID NO: 13; (d) a CDR-L1 with an amino acid sequence of SEQ ID NO: 14; (e) a CDR-L2 with an amino acid sequence of SEQ ID NO: 15; and (f) a CDR-L3 with an amino acid sequence of SEQ ID NO: 16. In some embodiments, the type I anti-CD20 antibody comprises a VH domain comprising an amino acid sequence of SEQ ID NO: 25 and a VL domain comprising an amino acid sequence of SEQ ID NO: 26. In some embodiments, the type I anti-CD20 antibody is rituximab.

In some embodiments of any of the third, fourth, and fifth aspects, the method further comprises administering to the patient an effective amount of an additional therapeutic agent. In some embodiments, the additional therapeutic agent is one or more of a chemotherapeutic agent, an anti-neoplastic agent, a growth inhibitory agent, an anti-angiogenic agent, a radiation therapy, a cytotoxic agent, or a combination thereof. In some embodiments, the additional therapeutic agent is a chemotherapeutic agent. In some embodiments, the chemotherapeutic agent is cyclophosphamide, doxorubicin, vincristine, or prednisone. In some embodiments, the chemotherapeutic agent is cyclophosphamide, doxorubicin, vincristine, and prednisone.

In some embodiments of any of the first, second, third, fourth, and fifth aspects, the patient has not been previously treated for the lymphoma.

In some embodiments of any of the first, second, third, fourth, and fifth aspects, the patient has not been previously administered an anti-CD20 antibody.

In a sixth aspect, the invention features a use of an anti-CD20 antibody for treating a patient having an amount or level of a macrophage biomarker in a sample from the patient that is above a reference macrophage biomarker amount or level in the manufacture of a medicament for the treatment of a lymphoma.

In some embodiments of the sixth aspect, the reference macrophage biomarker amount or level is a pre-assigned macrophage biomarker amount or level.

In some embodiments of the sixth aspect, the reference macrophage biomarker amount or level is an amount or level of a macrophage biomarker in a reference population. In some embodiments, the amount or level of the macrophage biomarker in a reference population is a median amount or level of the macrophage biomarker of the reference population. In some embodiments, the reference macrophage biomarker amount or level is an amount or level of a macrophage biomarker that is at the 25th percentile of the reference population. In some embodiments, the reference macrophage biomarker amount or level is an amount or level of a macrophage biomarker that is at the 50th percentile of the reference population. In some embodiments, the reference macrophage biomarker amount or level is an amount or level of a macrophage biomarker that is at the 75th percentile of the reference population. In some embodiments, the reference population is a population of patients having the lymphoma. In some embodiments, the population of patients having the lymphoma was previously treated with an anti-CD20 antibody. In some embodiments, the reference macrophage biomarker amount or level is the amount or level of macrophage biomarker of the reference population prior to initiating treatment with the anti-CD20 antibody. In some embodiments, the reference macrophage biomarker amount or level significantly separates the reference population into a first set of patients who have benefitted from the treatment with the anti-CD20 antibody and a second set of patients who have not benefitted from the treatment with the anti-CD20 antibody. In some embodiments, the amount of macrophages is between about 0% to about 30.7%.

In some embodiments of the sixth aspect, the treatment achieves an improvement of PFS or OS.

In some embodiments of the sixth aspect, the macrophage biomarker is an average of M1 macrophage gene signature set scores of one or more M1 macrophage gene signature sets. In some embodiments, each M1 macrophage gene signature set score is an average of the expression level of one or more genes of an M1 macrophage gene signature set. In some embodiments, each M1 macrophage gene signature set score is an average of the normalized expression level of one or more genes of an M1 macrophage gene signature set. In some embodiments, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some embodiments of the sixth aspect, the macrophage biomarker is an average of macrophage gene signature set scores of one or more macrophage gene signature sets. In some embodiments, each macrophage gene signature set score is an average of the expression level of one or more genes of a macrophage gene signature set. In some embodiments, each macrophage gene signature set score is an average of the normalized expression level of one or more genes of a macrophage gene signature set. In some embodiments, the one or more macrophage gene signature sets are any of the macrophage gene signature sets in Table 2.

In some embodiments of the sixth aspect, the macrophage biomarker is a gene expression value. In some embodiments, the gene expression value is a median gene expression value. In some embodiments, the gene expression value is measured using a gene signature matrix. In some embodiments, the gene signature matrix comprises the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1. In some embodiments, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some embodiments, the gene signature matrix is used to determine a number of macrophages. In some embodiments, the gene signature matrix is used to determine a number of M1 macrophages.

In some embodiments of the sixth aspect, the macrophage biomarker is an amount of M1 macrophages. In some embodiments, the amount of M1 macrophages is measured directly or indirectly. In some embodiments, the amount of M1 macrophages is measured directly using flow cytometry, spatial transcriptomics, spatial proteomics, or combination thereof. In some embodiments, the amount of M1 macrophages is measured indirectly using nucleic acid or protein. In some embodiments, the nucleic acid is measured using RNA-seq, RT-qPCR, qPCR, multiplex qPCR or RT-qPCR, microarray analysis, SAGE, MassARRAY technique, ISH, or a combination thereof. In some embodiments, the amount of M1 macrophages is measured using a marker gene approach or a deconvolution approach. In some embodiments, the marker gene approach uses xCell. In some embodiments, the deconvolution approach uses quanTlseq.

In some embodiments of the sixth aspect, the macrophage biomarker in the sample from the patient is measured using nucleic acid or protein. In some embodiments, the macrophage biomarker in the sample from the patient is determined using a nucleic acid expression level. In some embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some embodiments, the nucleic acid expression level is an mRNA expression level. In some embodiments, the mRNA expression level is determined by RNA-seq.

In some embodiments of the sixth aspect, the sample is a tissue sample, tumor sample, whole blood sample, a plasma sample, a serum sample, or a combination thereof. In some embodiments, the sample is a tissue sample. In some embodiments, the tissue sample is a tumor tissue sample. In some embodiments, the tumor tissue sample contains tumor cells, tumor-infiltrating immune cells, stromal cells, NAT cells, or a combination thereof. In some embodiments, the tumor tissue sample is a biopsy. In some embodiments, the sample is an archival sample, a fresh sample, or a frozen sample.

In some embodiments of the sixth aspect, the lymphoma is an indolent lymphoma.

In some embodiments of the sixth aspect, the lymphoma is a B-cell lymphoma. In some embodiments, the B-cell lymphoma is an NHL.

In some embodiments of the sixth aspect, the lymphoma is a DLBCL, an FL, a CLL, or an MZL. In some embodiments, the lymphoma is a DLBCL. In some embodiments, the DLBCL is a GCB or ABC cell-of-origin subgroup of DLBCL.

In some embodiments of the sixth aspect, the lymphoma is a CD20-positive lymphoma.

In some embodiments of the sixth aspect, the anti-CD20 antibody is a type I anti-CD20 antibody or a type II anti-CD20 antibody. In some embodiments, the anti-CD20 antibody is a type II anti-CD20 antibody. In some embodiments, the type II anti-CD20 antibody comprises the following complementarity determining regions (CDRs): (a) a CDR-H1 with an amino acid sequence of SEQ ID NO: 1; (b) a CDR-H2 with an amino acid sequence of SEQ ID NO: 2; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises the following CDRs: (a) a CDR-H1 with an amino acid sequence of SEQ ID NO: 27; (b) a CDR-H2 with an amino acid sequence of SEQ ID NO: 28; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises a VH domain comprising an amino acid sequence of SEQ ID NO: 7 and a VL domain comprising an amino acid sequence of SEQ ID NO: 8. In some embodiments, the type II anti-CD20 antibody is obinutuzumab. In some embodiments, the anti-CD20 antibody is a type I anti-CD20 antibody. In some embodiments, the type I anti-CD20 antibody comprises the following CDRs: (a) a CDR-H1 with an amino acid sequence of SEQ ID NO: 11; (b) a CDR-H2 with an amino acid sequence of SEQ ID NO: 12; (c) a CDR-H3 with an amino acid sequence of SEQ ID NO: 13; (d) a CDR-L1 with an amino acid sequence of SEQ ID NO: 14; (e) a CDR-L2 with an amino acid sequence of SEQ ID NO: 15; and (f) a CDR-L3 with an amino acid sequence of SEQ ID NO: 16. In some embodiments, the type I anti-CD20 antibody comprises a VH domain comprising an amino acid sequence of SEQ ID NO: 25 and a VL domain comprising an amino acid sequence of SEQ ID NO: 26. In some embodiments, the type I anti-CD20 antibody is rituximab.

In some embodiments of the sixth aspect, the medicament is to be administered to the patient in combination with an effective amount of an additional therapeutic agent. In some embodiments, the additional therapeutic agent is one or more of a chemotherapeutic agent, an anti-neoplastic agent, a growth inhibitory agent, an anti-angiogenic agent, a radiation therapy, a cytotoxic agent, or a combination thereof. In some embodiments, the additional therapeutic agent is a chemotherapeutic agent. In some embodiments, the chemotherapeutic agent is cyclophosphamide, doxorubicin, vincristine, or prednisone. In some embodiments, the chemotherapeutic agent is cyclophosphamide, doxorubicin, vincristine, and prednisone.

In some embodiments of the sixth aspect, the patient has not been previously treated for the lymphoma.

In some embodiments of the sixth aspect, the patient has not been previously administered an anti-CD20 antibody.

In a seventh aspect, the invention features an anti-CD20 antibody for use in the treatment of a patient having a lymphoma and having an amount or level of a macrophage biomarker in a sample from the patient that is above a reference macrophage biomarker amount or level. In some embodiments, the reference macrophage biomarker amount or level is a pre-assigned macrophage biomarker amount or level.

In some embodiments of the seventh aspect, the reference macrophage biomarker amount or level is an amount or level of a macrophage biomarker in a reference population. In some embodiments, the amount or level of the macrophage biomarker in a reference population is a median amount or level of the macrophage biomarker of the reference population. In some embodiments, the reference macrophage biomarker amount or level is an amount or level of a macrophage biomarker that is at the 25th percentile of the reference population. In some embodiments, the reference macrophage biomarker amount or level is an amount or level of a macrophage biomarker that is at the 50th percentile of the reference population. In some embodiments, the reference macrophage biomarker amount or level is an amount or level of a macrophage biomarker that is at the 75th percentile of the reference population. In some embodiments, the reference population is a population of patients having the lymphoma. In some embodiments, the population of patients having the lymphoma was previously treated with an anti-CD20 antibody. In some embodiments, the reference macrophage biomarker amount or level is the amount or level of macrophage biomarker of the reference population prior to initiating treatment with the anti-CD20 antibody. In some embodiments, the reference macrophage biomarker amount or level significantly separates the reference population into a first set of patients who have benefitted from the treatment with the anti-CD20 antibody and a second set of patients who have not benefitted from the treatment with the anti-CD20 antibody. In some embodiments of the seventh aspect, the reference macrophage biomarker amount or level is an amount of macrophages as measured by gene expression. In some embodiments, the amount of macrophages is between about 0% to about 30.7%.

In some embodiments of the seventh aspect, the treatment achieves an improvement of PFS or OS. In some embodiments of the seventh aspect, the macrophage biomarker is an average of M1 macrophage gene signature set scores of one or more M1 macrophage gene signature sets. In some embodiments, each M1 macrophage gene signature set score is an average of the expression level of one or more genes of an M1 macrophage gene signature set. In some embodiments, each M1 macrophage gene signature set score is an average of the normalized expression level of one or more genes of an M1 macrophage gene signature set. In some embodiments, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1 L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB31L1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some embodiments of the seventh aspect, the macrophage biomarker is an average of macrophage gene signature set scores of one or more macrophage gene signature sets. In some embodiments, each macrophage gene signature set score is an average of the expression level of one or more genes of a macrophage gene signature set. In some embodiments, each macrophage gene signature set score is an average of the normalized expression level of one or more genes of a macrophage gene signature set. In some embodiments, the one or more macrophage gene signature sets are any of the macrophage gene signature sets in Table 2.

In some embodiments of the seventh aspect, the macrophage biomarker is a gene expression value. In some embodiments, the gene expression value is a median gene expression value. In some embodiments, the gene expression value is measured using a gene signature matrix. In some embodiments, the gene signature matrix comprises the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1. In some embodiments, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1 PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some embodiments, the gene signature matrix is used to determine a number of macrophages. In some embodiments, the gene signature matrix is used to determine a number of M1 macrophages.

In some embodiments of the seventh aspect, the macrophage biomarker is an amount of M1 macrophages. In some embodiments, the amount of M1 macrophages is measured directly or indirectly. In some embodiments, the amount of M1 macrophages is measured directly using flow cytometry, spatial transcriptomics, spatial proteomics, or combination thereof. In some embodiments, the amount of M1 macrophages is measured indirectly using nucleic acid or protein. In some embodiments, the nucleic acid is measured using RNA-seq, RT-qPCR, qPCR, multiplex qPCR or RT-qPCR, microarray analysis, SAGE, MassARRAY technique, ISH, or a combination thereof. In some embodiments, the amount of M1 macrophages is measured using a marker gene approach or a deconvolution approach. In some embodiments, the marker gene approach uses xCell. In some embodiments, the deconvolution approach uses quanTlseq.

In some embodiments of the seventh aspect, the macrophage biomarker in the sample from the patient is measured using nucleic acid or protein. In some embodiments, the macrophage biomarker in the sample from the patient is determined using a nucleic acid expression level. In some embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some embodiments, the nucleic acid expression level is an mRNA expression level. In some embodiments, the mRNA expression level is determined by RNA-seq.

In some embodiments of the seventh aspect, the sample is a tissue sample, tumor sample, whole blood sample, a plasma sample, a serum sample, or a combination thereof. In some embodiments, the sample is a tissue sample. In some embodiments, the tissue sample is a tumor tissue sample. In some embodiments, the tumor tissue sample contains tumor cells, tumor-infiltrating immune cells, stromal cells, NAT cells, or a combination thereof. In some embodiments, the tumor tissue sample is a biopsy. In some embodiments, the sample is an archival sample, a fresh sample, or a frozen sample.

In some embodiments of the seventh aspect, the lymphoma is an indolent lymphoma.

In some embodiments of the seventh aspect, the lymphoma is a B-cell lymphoma. In some embodiments, the B-cell lymphoma is an NHL.

In some embodiments of the seventh aspect, the lymphoma is a DLBCL, an FL, a CLL, or an MZL. In some embodiments, the lymphoma is a DLBCL. In some embodiments, the DLBCL is a GCB or ABC cell-of-origin subgroup of DLBCL.

In some embodiments of the seventh aspect, the lymphoma is a CD20-positive lymphoma.

In some embodiments of the seventh aspect, the anti-CD20 antibody is a type I anti-CD20 antibody or a type II anti-CD20 antibody. In some embodiments, the anti-CD20 antibody is a type II anti-CD20 antibody. In some embodiments, the type II anti-CD20 antibody comprises the following complementarity determining regions (CDRs): (a) a CDR-H1 with an amino acid sequence of SEQ ID NO: 1; (b) a CDR-H2 with an amino acid sequence of SEQ ID NO: 2; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises the following CDRs: (a) a CDR-H1 with an amino acid sequence of SEQ ID NO: 27; (b) a CDR-H2 with an amino acid sequence of SEQ ID NO: 28; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises a VH domain comprising an amino acid sequence of SEQ ID NO: 7 and a VL domain comprising an amino acid sequence of SEQ ID NO: 8. In some embodiments, the type II anti-CD20 antibody is obinutuzumab. In some embodiments, the anti-CD20 antibody is a type I anti-CD20 antibody. In some embodiments, the type I anti-CD20 antibody comprises the following CDRs: (a) a CDR-H1 with an amino acid sequence of SEQ ID NO: 11; (b) a CDR-H2 with an amino acid sequence of SEQ ID NO: 12; (c) a CDR-H3 with an amino acid sequence of SEQ ID NO: 13; (d) a CDR-L1 with an amino acid sequence of SEQ ID NO: 14; (e) a CDR-L2 with an amino acid sequence of SEQ ID NO: 15; and (f) a CDR-L3 with an amino acid sequence of SEQ ID NO: 16. In some embodiments, the type I anti-CD20 antibody comprises a VH domain comprising an amino acid sequence of SEQ ID NO: 25 and a VL domain comprising an amino acid sequence of SEQ ID NO: 26. In some embodiments, the type I anti-CD20 antibody is rituximab.

In some embodiments of the seventh aspect, the treatment further comprises use of an effective amount of an additional therapeutic agent. In some embodiments, the additional therapeutic agent is one or more of a chemotherapeutic agent, an anti-neoplastic agent, a growth inhibitory agent, an anti-angiogenic agent, a radiation therapy, a cytotoxic agent, or a combination thereof. In some embodiments, the additional therapeutic agent is a chemotherapeutic agent. In some embodiments, the chemotherapeutic agent is cyclophosphamide, doxorubicin, vincristine, or prednisone. In some embodiments, the chemotherapeutic agent is cyclophosphamide, doxorubicin, vincristine, and prednisone.

In some embodiments of the seventh aspect, the patient has not been previously treated for the lymphoma.

In some embodiments of the seventh aspect, the patient has not been previously administered an anti-CD20 antibody.

In an eighth aspect, the invention features a method of identifying, diagnosing, and/or predicting whether a patient having a lymphoma may benefit from a treatment comprising an anti-CD20 antibody, the method comprising measuring a Th2 biomarker in a sample from the patient, wherein an amount or level of the Th2 biomarker in the sample that is above a reference Th2 biomarker amount or level identifies, diagnoses, and/or predicts the patient as one who may benefit from a treatment comprising an anti-CD20 antibody.

In a ninth aspect, the invention features a method of selecting a therapy for a patient having a lymphoma, the method comprising measuring a Th2 biomarker in a sample from the patient, wherein an amount or level of the Th2 biomarker in the sample that is above a reference Th2 biomarker amount or level identifies the patient as one who may benefit from a treatment comprising an anti-CD20 antibody.

In some embodiments of the eighth or ninth aspect, the patient has a Th2 biomarker in the sample that is above a reference Th2 biomarker amount or level, and the method further comprises administering to the patient an effective amount of an anti-CD20 antibody.

In a tenth aspect, the invention features a method of treating a patient having a lymphoma, the method comprising: (a) measuring a Th2 biomarker in a sample from the patient, wherein the amount or level of the Th2 biomarker in the sample is above a reference Th2 biomarker amount or level, and (b) administering an effective amount of an anti-CD20 antibody to the patient based on the Th2 biomarker measured in step (a).

In an eleventh aspect, the invention features a method of treating a patient having a lymphoma, the method comprising administering to the patient an effective amount of an anti-CD20 antibody, wherein prior to treatment the amount or level of a Th2 biomarker in a sample from the patient has been determined to be above a reference Th2 biomarker amount or level.

In a twelfth aspect, the invention features a method of treating a patient having a lymphoma and having an amount or level of a Th2 biomarker in a sample from the patient that is above a reference Th2 biomarker amount or level comprising administering to the patient an effective amount of an anti-CD20 antibody.

In some embodiments of any of the eighth, ninth, tenth, eleventh, and twelfth aspects, the reference Th2 biomarker amount or level is a pre-assigned Th2 biomarker amount or level.

In some embodiments of any of the eighth, ninth, tenth, eleventh, and twelfth aspects, the reference Th2 biomarker amount or level is an amount or level of a Th2 biomarker in a reference population. In some embodiments, the amount or level of the Th2 biomarker in a reference population is a median amount or level of the Th2 biomarker of the reference population. In some embodiments, the reference Th2 biomarker amount or level is an amount or level of a Th2 biomarker that is at the 25th percentile of the reference population. In some embodiments, the reference Th2 biomarker amount or level is an amount or level of a Th2 biomarker that is at the 50th percentile of the reference population. In some embodiments, the reference Th2 biomarker amount or level is an amount or level of a Th2 biomarker that is at the 75th percentile of the reference population. In some embodiments, the reference population is a population of patients having the lymphoma. In some embodiments, the population of patients having the lymphoma was previously treated with an anti-CD20 antibody. In some embodiments, the reference Th2 biomarker amount or level is the amount or level of Th2 biomarker of the reference population prior to initiating treatment with the anti-CD20 antibody. In some embodiments, the reference Th2 biomarker amount or level significantly separates the reference population into a first set of patients who have benefitted from the treatment with the anti-CD20 antibody and a second set of patients who have not benefitted from the treatment with the anti-CD20 antibody.

In some embodiments of any of the eighth, ninth, tenth, eleventh, and twelfth aspects, the reference Th2 biomarker amount or level is an amount of Th2 cells as measured by gene expression.

In some embodiments of the eighth or ninth aspect, the benefit is an extension of PFS.

In some embodiments of the eighth or ninth aspect, the benefit is an increase in OS.

In some embodiments of any of the tenth, eleventh, and twelfth aspects, comprising achieving an improvement of PFS or OS.

In some embodiments of any of the eighth, ninth, tenth, eleventh, and twelfth aspects, the Th2 biomarker is an average of a Th2 gene signature set scores of one or more Th2 gene signature sets. In some embodiments, each Th2 gene signature set score is an average of the expression level of one or more genes of a Th2 gene signature set. In some embodiments, each Th2 gene signature set score is an average of the normalized expression level of one or more genes of a Th2 gene signature set. In some embodiments, the one or more Th2 gene signature sets are any of the Th2 gene signature sets in Table 5.

In some embodiments of any of the eighth, ninth, tenth, eleventh, and twelfth aspects, the Th2 biomarker is an amount of Th2 cells. In some embodiments, the amount of Th2 cells is measured directly or indirectly. In some embodiments, the amount of Th2 cells is measured directly using flow cytometry, spatial transcriptomics, spatial proteomics, or combination thereof. In some embodiments, the amount of Th2 cells is measured indirectly using nucleic acid or protein. In some embodiments, the nucleic acid is measured using RNA-seq, RT-qPCR, qPCR, multiplex qPCR or RT-qPCR, microarray analysis, SAGE, MassARRAY technique, ISH, or a combination thereof. In some embodiments, the amount of Th2 cells is measured using a marker gene approach or a deconvolution approach. In some embodiments, the marker gene approach uses xCell. In some embodiments, the deconvolution approach uses quanTlseq.

In some embodiments of any of the eighth, ninth, tenth, eleventh, and twelfth aspects, the Th2 biomarker in the sample from the patient is measured using nucleic acid or protein. In some embodiments, the Th2 biomarker in the sample from the patient is determined using a nucleic acid expression level. In some embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some embodiments, the nucleic acid expression level is an mRNA expression level. In some embodiments, the mRNA expression level is determined by RNA-seq.

In some embodiments of any of the eighth, ninth, tenth, eleventh, and twelfth aspects, the sample is a tissue sample, tumor sample, whole blood sample, a plasma sample, a serum sample, or a combination thereof. In some embodiments, the sample is a tissue sample. In some embodiments, the tissue sample is a tumor tissue sample. In some embodiments, the tumor tissue sample contains tumor cells, tumor-infiltrating immune cells, stromal cells, NAT cells, or a combination thereof. In some embodiments, the tumor tissue sample is a biopsy. In some embodiments, the sample is an archival sample, a fresh sample, or a frozen sample.

In some embodiments of any of the eighth, ninth, tenth, eleventh, and twelfth aspects, the lymphoma is an indolent lymphoma.

In some embodiments of any of the eighth, ninth, tenth, eleventh, and twelfth aspects, the lymphoma is a B-cell lymphoma. In some embodiments, the B-cell lymphoma is a germinal center derived B-cell lymphoma. In some embodiments, the B-cell lymphoma is an NHL.

In some embodiments of any of the eighth, ninth, tenth, eleventh, and twelfth aspects, the lymphoma is a DLBCL, an FL, a CLL, or an MZL. In some embodiments, the lymphoma is a DLBCL. In some embodiments, the DLBCL is a GCB or ABC cell-of-origin subgroup of DLBCL.

In some embodiments of any of the eighth, ninth, tenth, eleventh, and twelfth aspects, the lymphoma is a CD20-positive lymphoma.

In some embodiments of any of the eighth, ninth, tenth, eleventh, and twelfth aspects, the anti-CD20 antibody is a type I anti-CD20 antibody or a type II anti-CD20 antibody. In some embodiments, the anti-CD20 antibody is a type II anti-CD20 antibody. In some embodiments, the type II anti-CD20 antibody comprises the following complementarity determining regions (CDRs): (a) a CDR-H1 with an amino acid sequence of SEQ ID NO: 1; (b) a CDR-H2 with an amino acid sequence of SEQ ID NO: 2; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises the following complementarity determining regions (CDRs): (a) a CDR-H1 with an amino acid sequence of SEQ ID NO: 27; (b) a CDR-H2 with an amino acid sequence of SEQ ID NO: 28; (c) a CDR-H3 with an amino acid sequence of SEQ ID NO: 3; (d) a CDR-L1 with an amino acid sequence of SEQ ID NO: 4; (e) a CDR-L2 with an amino acid sequence of SEQ ID NO: 5; and (f) a CDR-L3 with an amino acid sequence of SEQ ID NO: 6. In some embodiments, the type II anti-CD20 antibody comprises a VH domain comprising an amino acid sequence of SEQ ID NO: 7 and a VL domain comprising an amino acid sequence of SEQ ID NO: 8. In some embodiments, the type II anti-CD20 antibody is obinutuzumab. In some embodiments, the anti-CD20 antibody is a type I anti-CD20 antibody. In some embodiments, the type I anti-CD20 antibody comprises the following CDRs: (a) a CDR-H1 with an amino acid sequence of SEQ ID NO: 11; (b) a CDR-H2 with an amino acid sequence of SEQ ID NO: 12; (c) a CDR-H3 with an amino acid sequence of SEQ ID NO: 13; (d) a CDR-L1 with an amino acid sequence of SEQ ID NO: 14; (e) a CDR-L2 with an amino acid sequence of SEQ ID NO: 15; and (f) a CDR-L3 with an amino acid sequence of SEQ ID NO: 16. In some embodiments, the type I anti-CD20 antibody comprises a VH domain comprising an amino acid sequence of SEQ ID NO: 25 and a VL domain comprising an amino acid sequence of SEQ ID NO: 26. In some embodiments, the type I anti-CD20 antibody is rituximab.

In some embodiments of any of the tenth, eleventh, and twelfth aspects, further comprising administering to the patient an effective amount of an additional therapeutic agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing the study design of a global, open-label, randomized, Phase III clinical trial (NCT01287741) in first-line (1L) DLBCL patients. G-CHOP is obinutuzumab (G) plus cyclophosphamide, doxorubicin, vincristine, and prednisone (CHOP) and R-CHOP is rituximab (R) plus CHOP.

FIG. 2 is a pair of graphs showing improved survival in marker gene derived macrophage biomarker dichotomized patient subgroups. Investigator progression-free survival (PFS) (left) and overall survival (OS) (right) Kaplan-Meier Curves are shown for estimated high/low M1 macrophage signature samples leveraging xCell. Black denotes high M1 group whereas gray is low.

FIG. 3 is a table showing hazard ratios in marker gene derived (xCell) and deconvolution derived (Quantiseq) biomarker dichotomized patient subgroups. Biomarker cell subpopulations include macrophage, M1 macrophage, M2 macrophage, mast cell, and memory B cell. Univariate and multivariate forest plots are shown for macrophage high/low groups including hazard ratio and 95% confidence intervals. Left column are the associated statistics on investigator-assessed PFS. Right column are the associated statistics on OS.

FIG. 4 is a graph showing the association of lymphoma marker gene scores and PFS in xCell.

FIG. 5 is a graph showing the M1 macrophage composition present in patient samples as estimated by quanTlseq. Each bar represents the number of patient samples (y-axis) that have a M1 macrophage composition within a range (x-axis). The dashed line indicates the median value (0.03346) of M1 macrophage composition.

FIG. 6 is a heatmap showing immune deconvolution scores (scaled by column) generated by quanTlseq on 604 de novo DLBCL biopsies from patients treated with R/G-CHOP.

FIG. 7 is a pair of graphs showing improved survival in deconvolution derived macrophage biomarker dichotomized patient subgroups. Investigator PFS (left) and OS (right) Kaplan-Meier Curves are shown for estimated high/low M1 macrophage signature samples leveraging quanTlseq. Black denotes high M1 group whereas gray is low.

FIG. 8 is a graph showing the association of lymphoma infiltrating deconvolution scores and PFS in quanTlseq.

FIG. 9 is a graph showing PFS in patients with high versus low M1 macrophage enrichment based on quanTlseq.

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.” In some embodiments, “about” may refer to ±15%, ±10%, ±5%, or ±1% as understood by a person of skill in the art.

The “amount,” “level,” or “expression level,” used herein interchangeably, of a biomarker is a detectable level in a biological sample (e.g., a blood sample or a biopsy). “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., post-translational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who may be likely to respond to, or benefit from, a particular therapy (e.g., a therapy comprising one or more dosing cycles of an anti-CD20 antibody (e.g., obinutuzumab or rituximab)).

The presence and/or expression level/amount of various biomarkers described herein in a sample (e.g., a blood sample or a biopsy) can be analyzed by a number of methodologies, many of which are known in the art and understood by the skilled artisan, including, but not limited to, immunohistochemistry (“IHC”), western blot analysis, immunoprecipitation, molecular binding assays, ELISA, ELIFA, flow cytometry, fluorescence activated cell sorting (“FACS”), spatial transcriptomics, spatial proteomics, MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The meaning of “Type I” and “Type II” anti-CD20 antibodies are well known in the art. In general, anti-CD20 monoclonal antibodies fall into two distinct categories based on their mechanism of action in eradicating lymphoma cells. “Type I” anti-CD20 antibodies primarily utilize complement to kill target cells, while “Type II” anti-CD20 antibodies operate by different mechanisms, primarily apoptosis. Rituximab (see, e.g., U.S. Pat. No. 5,736,137, which is incorporated herein by reference in its entirety) and 1F5 are examples of Type I anti-CD20 antibodies, whereas obinutuzumab (see, e.g., WO 2005/044859 and U.S. Patent Publication No. 2005/0123546, which are incorporated by reference herein in their entirety) and B1 are examples of a Type II antibody. See, e.g., Cragg (Blood 103(7), 2004, 2738-2743); Teeling (Blood 104(6), 2004, 1793-1800); EP2380910 and WO 2005/044859, the entire contents of which are hereby incorporated by reference.

As used herein, “administering” is meant a method of giving a dosage of a compound (e.g., an anti-CD20 antibody (e.g., obinutuzumab or rituximab)), or a composition (e.g., a pharmaceutical composition, e.g., a pharmaceutical composition including an anti-CD20 antibody (e.g., obinutuzumab or rituximab)) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., an anti-CD20 antibody (e.g., obinutuzumab or rituximab)) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual. The co-administration can be simultaneous or sequential in either order, wherein preferably there is a time period while both or all active agents simultaneously exert their biological activities. Said antibody and said further agent(s) are co-administered either simultaneously or sequentially (e.g., intravenous (i.v.)), for example through a continuous infusion. When both therapeutic agents are co-administered sequentially the dose administered either on the same day in two separate administrations, or one of the agents may be administered on day 1 and the second may be co-administered on day 2 to day 7, preferably on day 2 to 4. Thus, in one embodiment, the term “sequentially” means within (about) 7 days after the dose of the first component, preferably within (about) 4 days after the dose of the first component; and the term “simultaneously” means at the same time. The term “coadministration” with respect to the maintenance doses of the antibody and/or further agent(s) means that the maintenance doses can be either co-administered simultaneously, if the treatment cycle is appropriate for both drugs, e.g., every week or the further agent is, e.g., administered, e.g., every first to third day and said antibody is administered every week. Or the maintenance doses are co-administered sequentially, either within one or within several days. In a preferred embodiment, the anti-CD20 antibodies described herein (e.g., obinutuzumab, rituximab, or their functional equivalents) may be administered in combination with a chemotherapy, for example with a CHOP chemotherapy or with variants of a CHOP chemotherapy (e.g., a CHOEP chemotherapy, a CHOP-14 chemotherapy or an ACVBP chemotherapy (see, for example, the examples and also EP-B1 2380910, WO 2005/044859 and Scott, 2014 and 2015, loc. cit.)). Therefore, in a preferred embodiment, the additional chemotherapeutic agents to be co-administered are selected from the group consisting of cyclophosphamide, hydroxydaunorubicin, oncovein, prednisone or prednisolone and, optionally, etoposide.

A “disorder” or “disease” is any condition that would benefit from treatment including, but not limited to, disorders that are associated with some degree of abnormal cell proliferation, e.g., cancer, e.g., lymphoma.

The terms “cancer” and “cancerous” refer to or describe the physiological condition in mammals that is typically characterized by unregulated cell growth. Examples of cancer include, but are not limited to, lymphoma, carcinoma, blastoma, sarcoma, and leukemia or lymphoid malignancies. More particular examples of such cancers include, but are not limited to, multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), and associated metastases. In some embodiments, the cancer is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some embodiments, the lymphoma is an indolent lymphoma. In some embodiments, the lymphoma is a B-cell lymphoma. In some embodiments, the B-cell lymphoma is a germinal center derived B-cell lymphoma. In some embodiments, the B-cell lymphoma is an NHL. In some embodiments, the lymphoma is a diffuse large B-cell lymphoma (DLBCL), a follicular lymphoma (FL), a chronic lymphocytic leukemia (CLL), or a marginal zone lymphoma (MZL). In some embodiments, the lymphoma is a DLBCL. In some embodiments, the DLBCL is a germinal-center B-cell-like (GCB) or activated B-cell-like (ABC) cell-of-origin subgroup of DLBCL. In some embodiments, the lymphoma is a CD20-positive lymphoma. In some embodiments, the lymphoma is a marginal zone lymphoma (e.g., an extranodal, nodal, or splenic marginal zone lymphoma).

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

As used herein, “metastasis” is meant the spread of cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). Examples of anti-cancer therapeutic agents include, but are limited to, e.g., immunomodulatory agents, or an agent that increases or activates one or more immune co-stimulatory receptors, chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention. In some embodiments, the anti-cancer therapy includes cyclophosphamide, doxorubicin, vincristine, and prednisone (CHOP) or variants thereof (e.g., a CHOEP chemotherapy, a CHOP-14 chemotherapy or an ACVBP chemotherapy (see, for example, the examples and also EP-B1 2380910, WO 2005/044859 and Scott, 2014 and 2015, loc. cit.))

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate , salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1| and calicheamicin ω1| (Angew Chem. Intl. Ed. Engl. 1994 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene , 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds described include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti—interleukin-12 (ABT-874/J695, Wyeth Research and Abbott Laboratories) which is a recombinant exclusively human-sequence, full-length IgG1 A antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to

EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29): 30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos: 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl], dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, 1131, 1125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone (prednisone); and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-CD20 antibody (e.g., obinutuzumab or rituximab)), or a composition (e.g., pharmaceutical composition) thereof, is at least the minimum amount required to achieve the desired therapeutic result, such as a measurable increase in overall survival or progression-free survival of a particular disease or disorder (e.g., lymphoma, e.g., a B-cell lymphoma, e.g., a on-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), increase from baseline in functional assessment of cancer therapy-lymphoma (FACT-Lym) subscale score, decreasing the dose of other medications required to treat the disease, enhancing effect of another medication such as via targeting, delaying the progression of the disease (e.g., progression-free survival, delay of unequivocal clinical progression (e.g., cancer-related pain progression, deterioration in Eastern Cooperative Group Oncology Group (ECOG) Performance Status (PS) (e.g., how the disease affects the daily living abilities of the patient), and/or initiation of next systemic anti-cancer therapy, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with an anti-CD20 antibody (e.g., obinutuzumab or rituximab).

“Individual response” or “response” can be assessed using any endpoint indicating a benefit to the subject, including, without limitation, (1) inhibition, to some extent, of disease progression (e.g., progression of cancer, e.g., lymphoma) including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)); (6) increase or extend in the length of survival, including overall survival and progression-free survival; and/or (9) decreased mortality at a given point of time following treatment.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the time from entry into a study to death from any cause. As used herein, “overall survival rate” refers to the percentage of subjects in a group who are alive after a particular duration of time, e.g., six months, 1 year, or 5 years from the time of diagnosis or treatment.

As used herein, “complete response” or “CR” refers to disappearance of all evidence of disease. As used herein, “partial response” or “PR” refers to a measurable alleviation of symptoms, diminishment of any direct or indirect pathological consequences of the lymphoma, decrease in the rate of disease progression, amelioration or palliation of the disease state, or prevention of metastasis that does not eliminate all evidence of disease.

As used herein, “progression-free survival” (PFS) refers to the length of time during and after treatment during which the disease being treated (e.g., cancer, e.g., e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) does not get worse (e.g., lymphoma progression or death as a result of any cause). PFS may include the amount of time patients have experienced a complete response or a partial response, as well as the amount of time patients have experienced stable disease.

As used herein “extending survival” refers to increasing overall survival or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

The skilled person is readily able to decide whether a given clinical outcome is improved in accordance with the invention (e.g., improved as compared to a treatment without an anti-CD20 antibody (e.g., obinutuzumab or rituximab)). For example, “improved” in this context means that the clinical outcome (resulting from the treatment with the anti-CD20 antibody (e.g., obinutuzumab/a functional equivalent of obinutuzumab or rituximab), particularly in combination with a chemotherapy, particularly in combination with a CHOP chemotherapy) is at least 3% higher, at least 5% higher, at least 7% higher, at least 10% higher, at least 15% higher, at least 20% higher, at least 25% higher, at least 30% higher, at least 40% higher, at least 50% higher, at least 75% higher, at least 100% higher, or at least 120% higher, as compared to the clinical outcome resulting from a comparable treatment without an anti-CD20 antibody (e.g., obinutuzumab or rituximab), particularly in combination with a chemotherapy, particularly in combination with a CHOP chemotherapy.

The time at which the clinical outcome/clinical endpoint is assessed can readily be determined by the skilled person. In principle, it is determined at a timepoint when the difference in the clinical outcome/clinical endpoint between the two treatments (e.g., obinutuzumab treatment vs. rituximab treatment) becomes evident. This time may, for example, be at least 1 month, at least 2 months, at least 3 months, at least 6 months, at least 12 months, at least 18 months, at least 24 months, at least 30 months, at least 36 months, at least 42 months, or at least 48 months, after the beginning of the treatment.

As used herein, “delaying progression” of a disorder or disease means to defer, hinder, slow, retard, stabilize, and/or postpone development of the disease or disorder (e.g., cancer, e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). This delay can be of varying lengths of time, depending on the history of the disease and/or subject being treated. As is evident to one skilled in the art, a sufficient or significant delay can, in effect, encompass prevention, in that the subject does not develop the disease.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the presence or size of metastases, or the size of the primary tumor.

As used herein, “subject” or “individual” is meant a mammal, including, but not limited to, a human or non-human mammal, such as a bovine, equine, canine, ovine, or feline. In some embodiments, the subject is a human. Patients are also humans herein.

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, macrophages (e.g., M1 macrophages or M2 macrophages), monocytes, T lymphocytes (such as CD8+ T lymphocytes and/or CD4+ T lymphocytes), B lymphocytes, or other bone marrow-lineage cells, including granulocytes (e.g., neutrophils, eosinophils, and basophils), dendritic cells (e.g., interdigitating dendritic cells), histiocytes, and natural killer cells.

The term “biomarker” as used herein refers to an indicator, e.g., predictive, diagnostic, and/or prognostic, which can be detected in a sample (e.g., a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample), a blood sample, or a biopsy). The biomarker may serve as an indicator of a particular subtype of a disease or disorder (e.g., a diffuse large B-cell lymphoma (e.g., a germinal-center B-cell-like or activated B-cell-like diffuse large B-cell lymphoma)) characterized by certain, molecular, pathological, histological, and/or clinical features. In some aspects, a biomarker is a gene (e.g., any of the genes described herein). Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers. In some embodiments, the biomarker is a gene expression value. In some embodiments, the biomarker is a M1 macrophage gene signature set score. In some embodiments, the biomarker is a cell (e.g., an immune cell, e.g., a macrophage, e.g., an M1 macrophage or an M2 macrophage). In some embodiments, the biomarker is an amount of macrophages. In some embodiments, the biomarker is an amount of M1 macrophages.

The term “macrophage biomarker” as used herein refers to a biomarker that indicates an amount, level, characteristic, or phenotype of macrophages within a sample (e.g., a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample), a blood sample, or a biopsy). In some aspects, a macrophage biomarker is a gene (e.g., any of the genes described herein). In some aspects, a macrophage biomarker is a polypeptide, polynucleotide (e.g., DNA, and/or RNA), polynucleotide copy number alteration (e.g., DNA copy number), polypeptide and polynucleotide modification (e.g., posttranslational modification), carbohydrate, and/or glycolipid-based molecular marker. In some embodiments, the macrophage biomarker is a gene expression value, which can be reflective of one or more genes (e.g., one or more of the genes described herein). In some embodiments, the macrophage biomarker is a M1 macrophage gene signature set score. In some embodiments, the macrophage biomarker is a cell (e.g., an immune cell, e.g., a macrophage, e.g., an M1 macrophage or an M2 macrophage). In some embodiments, the macrophage biomarker is an amount of macrophages (e.g., an amount of M1 macrophages or an amount of M2 macrophages). In some embodiments, the macrophage biomarker is an amount of M1 macrophages.

The term “Th2 biomarker” as used herein refers to a biomarker that indicates an amount, level, characteristic, or phenotype of CD4+Type 2 helper T cells within a sample (e.g., a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample), a blood sample, or a biopsy). In some aspects, a Th2 biomarker is a gene. In some aspects, a Th2 biomarker is a polypeptide, polynucleotide (e.g., DNA, and/or RNA), polynucleotide copy number alteration (e.g., DNA copy number), polypeptide and polynucleotide modification (e.g., posttranslational modification), carbohydrate, and/or glycolipid-based molecular marker. In some aspects, a Th2 biomarker is a gene expression value, which can be reflective of one or more genes. In some aspects, a Th2 biomarker is a gene signature set score. In some embodiments, a Th2 biomarker is a cell (e.g., an immune cell, e.g., a T cell, e.g., a helper T cell, e.g., a Th2 cell). In some embodiments, a Th2 biomarker is an amount of T cells (e.g., an amount of Th2 cells). In some embodiments, a Th2 biomarker is an amount of Th2 cells.

A biomarker is “predictive” in accordance with the invention if it can be used to identify a patient defined herein (optionally in combination with one or more other biomarkers), e.g., a patient that responds to treatment with an anti-CD20 antibody (e.g., obinutuzumab or rituximab) (particularly in combination with a chemotherapy, particularly in combination with a CHOP chemotherapy). In some embodiments, a biomarker is predictive if the treatment effect differs between the biomarker-defined subgroups of patients. It is preferred in this context, that the predictive biomarker(s) is (are) the biomarker(s) as defined herein elsewhere. Particular examples of predictive biomarkers to be assessed in the context of the invention are the macrophage biomarkers described herein.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)2, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, β, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” as used herein refers to each of the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13: 37-45 (2000); Johnson and Wu, in Methods in Molecular Biology 248: 1-25 (Lo, ed., Human Press, Totowa, NJ, 2003). Indeed, naturally occurring camelid antibodies consisting of a heavy chain only are functional and stable in the absence of light chain. See, e.g., Hamers-Casterman et al., Nature 363: 446-448 (1993); Sheriff et al., Nature Struct. Biol. 3: 733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196: 901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. “Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab', F(ab')2 and Fv fragments; diabodies; linear antibodies (see U.S. Pat. No. 5,641,870, Example 2; Zapata et al., Protein Eng. 8(10): 1057-1062 (1995)); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies described comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors, FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. (see M. Daĕron, Annu. Rev. Immunol. 15: 203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., CD20). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described in the following.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227: 381 (1991); Marks et al., J. Mol. Biol., 222: 581 (1991). Also available for the preparation of human monoclonal antibodies are methods described in Cole et al., Monoclonal Antibodies and Cancer Therapy, Alan R. Liss, p. 77 (1985); Boerner et aL, J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103: 3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321: 522-525 (1986); Riechmann et al., Nature 332: 323-329 (1988); and Presta, Curr. Op. Struct. Biol. 2: 593-596 (1992). See also, for example, Vaswani and Hamilton, Ann. Allergy, Asthma & Immunol. 1: 105-115 (1998); Harris, Biochem. Soc. Transactions 23: 1035-1038 (1995); Hurle and Gross, Curr. Op. Biotech. 5: 428-433 (1994); and U.S. Pat. Nos. 6,982,321 and 7,087,409.

The term “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848: 79-87 (2007). In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256: 495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 1⁰⁻4M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The phrase “substantially reduced” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{D} values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., K_{D} values). The difference between said two values is, for example, less than about 50%, less than about 40%, less than about 30%, less than about 20%, and/or less than about 10% as a function of the reference/comparator value.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
   where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample (e.g., a biopsy or a blood sample) obtained from a subject of interest that would be expected or is known to contain the cellular and/or molecular entity that is to be characterized. In some embodiments, the sample is a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample). Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof. Samples may be fresh or may be processed (e.g., frozen, fixed, or formalin-fixed, paraffin-embedded (FFPE)) for storage.

By “tissue sample” or “cell sample” is meant a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a diseased tissue/organ. The tissue sample may contain compounds which are not naturally intermixed with the tissue in nature such as preservatives, anticoagulants, buffers, fixatives, wax, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

In general, as used in the context of the present invention, a non-limiting example of a “control” is preferably a “non-responder” control, for example a sample/cell/tissue obtained from one or more patients that do not suffer from the particular lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) as defined herein (non-“patient defined herein”) and that are known to be not advantageously responsive to an anti-CD20 antibody (e.g., obinutuzumab or rituximab) (in particular in combination with a chemotherapy, more particular in combination with a CHOP chemotherapy) in accordance with the invention. Another example for a “non-responder” control is a cell line/sample/cell/tissue that shows no improved response to an anti-CD20 antibody (e.g., obinutuzumab or rituximab) (particularly in combination with a chemotherapy, particularly in combination with a CHOP chemotherapy) in an ex-vivo test. Another non-limiting example of a “control” is an “internal standard”, for example purified or synthetically produced proteins, peptides, DNA and/or RNA, or a mixture thereof, where the amount of each protein/peptide/DNA/RNA is gauged by using the “non-responder” control described herein. In principle, the patient to be treated in the context of the invention is envisaged to be a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) patient. In other words, the patient is a patient with/suffering from lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). Accordingly, it is particularly envisaged that also the patient defined with respect to any of the aspects/embodiments is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) patient and a patient with/suffering from lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), respectively. However, it is not necessarily required that a given patient is diagnosed as being a lymphoma patient, for example prior to (or after) the determination/identification/diagnosis of being a patient as defined herein, in particular as defined in one or more of the aspects/embodiments. It is, however, preferred that the patient to be treated in accordance with the invention is, in a first step, diagnosed as being a lymphoma patient and, in a second step, determined/identified/diagnosed as being a patient defined herein, in particular a patient as defined in one or more of the aspects/embodiments. In principle, in accordance with the invention, a given patient may, in a first step, also be determined/identified/diagnosed as being a patient defined herein, and, in a second step, diagnosed as being a lymphoma patient. However, the latter option is less preferred and, as mentioned, the (foregoing or subsequent) step of diagnosing whether the patient to be treated is a lymphoma (e.g., DLBCL) patient may also be omitted.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

An “article of manufacture” is any manufacture (e.g., a package or container) or kit comprising at least one reagent, e.g., a medicament for treatment of a disease or disorder (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), and a package insert. In certain embodiments, the manufacture or kit is promoted, distributed, or sold as a unit for performing the methods described herein.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

### III. Diagnostic Methods and Assays

Provided herein are methods and assays for identifying, diagnosing, and/or predicting whether a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) may benefit from a treatment comprising an anti-CD20 antibody (e.g., obinutuzumab or rituximab). The methods and assays described herein are based on the finding that an amount or level of a macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) or a Th2 biomarker (e.g., an amount of Th2 cells) in a sample (e.g., a tissue sample, e.g., a tumor tissue sample, such as a biopsy) from the patient may be used to identify, diagnose, and/or predict the patient as one who may benefit from the treatment comprising an anti-CD20 antibody (e.g., obinutuzumab or rituximab). Any of the methods provided herein may further include administering to the patient an anti-CD20 antibody (e.g., obinutuzumab or rituximab).

### A. Macrophage Biomarkers

In particular instances, the methods and assays provided herein may be used to determine the amount or level of a macrophage biomarker. Various diagnostic methods based on a determination of the amount or level of the macrophage biomarker are further described below.

In one aspect, provided herein are methods for identifying, diagnosing, and/or predicting whether a patient having a lymphoma may benefit from a treatment comprising an anti-CD20 antibody, the method including measuring a macrophage biomarker in a sample from the patient, wherein an amount or level of the macrophage biomarker in the sample that is above a reference macrophage biomarker amount or level identifies, diagnoses, and/or predicts the patient as one who may benefit from a treatment comprising an anti-CD20 antibody. In some instances, the methods further comprise administering an anti-CD20 antibody. Alternatively, an amount or level of the macrophage biomarker in the sample that is below a reference macrophage biomarker amount or level identifies, diagnoses, and/or predicts the patient as one who may not benefit from a treatment comprising an anti-CD20 antibody.

In another aspect, provided herein are methods for selecting a therapy for a patient having a lymphoma, the method including measuring a macrophage biomarker in a sample from the patient, wherein an amount or level of the macrophage biomarker in the sample that is above a reference macrophage biomarker amount or level identifies the patient as one who may benefit from a treatment comprising an anti-CD20 antibody. In some instances, the methods further comprise administering an anti-CD20 antibody. Alternatively, an amount or level of the macrophage biomarker in the sample that is below a reference macrophage biomarker amount or level identifies the patient as one who may not benefit from a treatment comprising an anti-CD20 antibody.

### (i) Increased Macrophage Biomarker A

n amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is above a reference macrophage biomarker amount or level may identify, diagnose, and/or predict the patient as one who may benefit from a treatment comprising an anti-CD20 antibody (e.g., obinutuzumab or rituximab).

For example, in some instances, an amount or level of the macrophage biomarker in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85th percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the macrophage biomarker in the reference population identifies the individual as one who may benefit from a treatment including an anti-CD20 antibody (e.g., obinutuzumab or rituximab).

In some instances, an amount or level of the macrophage biomarker in the sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the macrophage biomarker in the reference population identifies the individual as one who may benefit from a treatment including an anti-CD20 antibody (e.g., obinutuzumab or rituximab). For example, in some instances, an amount or level of the macrophage biomarker in the sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who may benefit from a treatment including an anti-CD20 antibody (e.g., obinutuzumab or rituximab).

In some instances, an amount or level of the macrophage biomarker in the sample that is in about the top 80th percentile (i.e., equal to, or higher than, the 20% prevalence level) of the reference population identifies the individual as one who may benefit from a treatment including an anti-CD20 antibody (e.g., obinutuzumab or rituximab). In some instances, an amount or level of the macrophage biomarker in the sample that is in about the top 75th percentile (i.e., equal to, or higher than, the 25% prevalence level) of the reference population identifies the individual as one who may benefit from a treatment including an anti-CD20 antibody (e.g., obinutuzumab or rituximab). In some instances, an amount or level of the macrophage biomarker in the sample that is in about the top 50th percentile (i.e., equal to, or higher than, the 50% prevalence level) of the reference population identifies the individual as one who may benefit from a treatment including an anti-CD20 antibody (e.g., obinutuzumab or rituximab). In some instances, an amount or level of the macrophage biomarker in the sample that is in about the top 25th percentile (i.e., equal to, or higher than, the 75% prevalence level) of the reference population identifies the individual as one who may benefit from a treatment including an anti-CD20 antibody (e.g., obinutuzumab or rituximab). In some instances, an amount or level of the macrophage biomarker in the sample that is in about the top 20th percentile (i.e., equal to, or higher than, the 80% prevalence level) of the reference population identifies the individual as one who may benefit from a treatment including an anti-CD20 antibody (e.g., obinutuzumab or rituximab).

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Decreased Macrophage Biomarker

An amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is below a reference macrophage biomarker amount or level may identify, diagnose, and/or predict the patient as one who may not benefit from a treatment comprising an anti-CD20 antibody (e.g., obinutuzumab or rituximab).

In some instances, an amount or level of the macrophage biomarker in the sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence 5 level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population identifies the individual as one who is less likely to benefit from a treatment including an anti-CD20 antibody (e.g., obinutuzumab or rituximab).

In some instances, an amount or level of the macrophage biomarker in the sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population identifies the individual as one who is less likely to benefit from a treatment including an anti-CD20 antibody (e.g., obinutuzumab or rituximab). For example, in some instances, an amount or level of the macrophage biomarker in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who is less likely to benefit from a treatment including an anti-CD20 antibody (e.g., obinutuzumab or rituximab).

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (iii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%.

In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes two or more (e.g., two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iv) Indications

The methods described herein are useful for predicting the therapeutic response of an individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) to treatment with an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for identifying, diagnosing, and/or predicting whether a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) may benefit from a treatment comprising an anti-CD20 antibody (e.g., obinutuzumab or rituximab), the method including measuring a macrophage biomarker in a sample from the patient, wherein an amount or level of the macrophage biomarker in the sample that is above a reference macrophage biomarker amount or level identifies, diagnoses, and/or predicts the patient as one who may benefit from a treatment comprising an anti-CD20 antibody. Alternatively, an amount or level of the macrophage biomarker in the sample that is below a reference macrophage biomarker amount or level identifies, diagnoses, and/or predicts the patient as one who may not benefit from a treatment comprising an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (v) Treatment Benefits

A patient who benefits from receiving treatment with an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in overall survival (OS), progression-free survival (PFS), complete response (CR), partial response (PR), or a combination thereof. In some instances, an amount or level of a macrophage biomarker that is above a reference macrophage biomarker amount or level (e.g., an amount or level of a macrophage biomarker in a reference population) identifies the individual as one who may benefit from a treatment including an anti-CD20 antibody (e.g., obinutuzumab or rituximab), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, an amount or level of a macrophage biomarker that is above a reference macrophage biomarker amount or level (e.g., an amount or level of a macrophage biomarker in a reference population) identifies the individual as one who may benefit from a treatment including an anti-CD20 antibody (e.g., obinutuzumab or rituximab), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### B. Th2 Biomarkers

In particular instances, the methods and assays provided herein may be used to determine the amount or level of a Th2 biomarker. Various diagnostic methods based on a determination of the amount or level of the Th2 biomarker are further described below.

In one aspect, provided herein are methods for identifying, diagnosing, and/or predicting whether a patient having a lymphoma may benefit from a treatment comprising an anti-CD20 antibody, the method including measuring a Th2 biomarker in a sample from the patient, wherein an amount or level of the Th2 biomarker in the sample that is above a reference Th2 biomarker amount or level identifies, diagnoses, and/or predicts the patient as one who may benefit from a treatment comprising an anti-CD20 antibody. In some instances, the methods further comprise administering an anti-CD20 antibody. Alternatively, an amount or level of the Th2 biomarker in the sample that is below a reference Th2 biomarker amount or level identifies, diagnoses, and/or predicts the patient as one who may not benefit from a treatment comprising an anti-CD20 antibody.

In another aspect, provided herein are methods for selecting a therapy for a patient having a lymphoma, the method including measuring a Th2 biomarker in a sample from the patient, wherein an amount or level of the Th2 biomarker in the sample that is above a reference Th2 biomarker amount or level identifies the patient as one who may benefit from a treatment comprising an anti-CD20 antibody. In some instances, the methods further comprise administering an anti-CD20 antibody. Alternatively, an amount or level of the Th2 biomarker in the sample that is below a reference Th2 biomarker amount or level identifies the patient as one who may not benefit from a treatment comprising an anti-CD20 antibody.

### (i) Increased Th2 Biomarker

An amount or level of the Th2 biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is above a reference Th2 biomarker amount or level may identify, diagnose, and/or predict the patient as one who may benefit from a treatment comprising an anti-CD20 antibody (e.g., obinutuzumab or rituximab).

For example, in some instances, an amount or level of the Th2 biomarker in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the Th2 biomarker in the reference population identifies the individual as one who may benefit from a treatment including an anti-CD20 antibody (e.g., obinutuzumab or rituximab).

In some instances, an amount or level of the Th2 biomarker in the sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the Th2 biomarker in the reference population identifies the individual as one who may benefit from a treatment including an anti-CD20 antibody (e.g., obinutuzumab or rituximab). For example, in some instances, an amount or level of the Th2 biomarker in the sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who may benefit from a treatment including an anti-CD20 antibody (e.g., obinutuzumab or rituximab).

In some instances, an amount or level of the Th2 biomarker in the sample that is in about the top 80th percentile (i.e., equal to, or higher than, the 20% prevalence level) of the reference population identifies the individual as one who may benefit from a treatment including an anti-CD20 antibody (e.g., obinutuzumab or rituximab). In some instances, an amount or level of the Th2 biomarker in the sample that is in about the top 75th percentile (i.e., equal to, or higher than, the 25% prevalence level) of the reference population identifies the individual as one who may benefit from a treatment including an anti-CD20 antibody (e.g., obinutuzumab or rituximab). In some instances, an amount or level of the Th2 biomarker in the sample that is in about the top 50th percentile (i.e., equal to, or higher than, the 50% prevalence level) of the reference population identifies the individual as one who may benefit from a treatment including an anti-CD20 antibody (e.g., obinutuzumab or rituximab). In some instances, an amount or level of the Th2 biomarker in the sample that is in about the top 25th percentile (i.e., equal to, or higher than, the 75% prevalence level) of the reference population identifies the individual as one who may benefit from a treatment including an anti-CD20 antibody (e.g., obinutuzumab or rituximab). In some instances, an amount or level of the Th2 biomarker in the sample that is in about the top 20th percentile (i.e., equal to, or higher than, the 80% prevalence level) of the reference population identifies the individual as one who may benefit from a treatment including an anti-CD20 antibody (e.g., obinutuzumab or rituximab).

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to the amount or level of the Th2 biomarker in a reference sample, reference cell, reference tissue, control sample, control cell, or control tissue. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the Th2 biomarker in a reference sample, reference cell, reference tissue, control sample, control cell, or control tissue. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the Th2 biomarker in a reference sample, reference cell, reference tissue, control sample, control cell, or control tissue.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to a pre-assigned amount or level of the Th2 biomarker. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the Th2 biomarker. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the Th2 biomarker.

### (ii) Reference Th2 Biomarker

The reference Th2 biomarker amount or level can be a pre-assigned Th2 biomarker amount or level. In some instances, the amount or level of the Th2 biomarker in a reference population is a median amount or level of the Th2 biomarker of the reference population. In some instances, the amount or level of the Th2 biomarker in a reference population is a mean amount or level of the Th2 biomarker of the reference population.

In some instances, the pre-assigned Th2 biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19% , 20% , 21%, 22%, 23%, 24% , 25% , 26%, 27%, 28% , 29% , 30% , 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 0%.

The reference amount or level of the Th2 biomarker described herein may be based on the amount or level of the Th2 biomarker in a reference population. In some instances, the reference Th2 biomarker described herein is an amount or level of the Th2 biomarker in a reference population that includes two or more (e.g., two or more, three or more, four or more, or five or more) subsets of patients.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the Th2 biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the Th2 biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference Th2 biomarker may be an amount or level of the Th2 biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iii) Indications

The methods described herein are useful for predicting the therapeutic response of an individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) to treatment with an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for identifying, diagnosing, and/or predicting whether a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) may benefit from a treatment comprising an anti-CD20 antibody (e.g., obinutuzumab or rituximab), the method including measuring a Th2 biomarker in a sample from the patient, wherein an amount or level of the Th2 biomarker in the sample that is above a reference Th2 biomarker amount or level identifies, diagnoses, and/or predicts the patient as one who may benefit from a treatment comprising an anti-CD20 antibody. Alternatively, an amount or level of the Th2 biomarker in the sample that is below a reference Th2 biomarker amount or level identifies, diagnoses, and/or predicts the patient as one who may not benefit from a treatment comprising an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (iv) Treatment Benefits

A patient who benefits from receiving treatment with an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in overall survival (OS), progression-free survival (PFS), complete response (CR), partial response (PR), or a combination thereof.

In some instances, an amount or level of a Th2 biomarker that is above a reference Th2 biomarker amount or level (e.g., an amount or level of a Th2 biomarker in a reference population) identifies the individual as one who may benefit from a treatment including an anti-CD20 antibody (e.g., obinutuzumab or rituximab), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, an amount or level of a Th2 biomarker that is above a reference Th2 biomarker amount or level (e.g., an amount or level of a Th2 biomarker in a reference population) identifies the individual as one who may benefit from a treatment including an anti-CD20 antibody (e.g., obinutuzumab or rituximab), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### C. Determination of Macrophage Biomarkers

### (i) Detection Methods

The macrophage biomarkers described herein may be based on an amount or expression level of a nucleic acid (e.g., an mRNA), a protein, or a cell (e.g., a macrophage, e.g., an M1 macrophage). Presence and/or expression levels/amount of the genes described herein (see, e.g., Tables 1-4) can be determined qualitatively and/or quantitatively based on any suitable criterion known in the art, including but not limited to DNA, mRNA, cDNA, proteins, protein fragments, and/or gene copy number. Presence and/or levels/amounts of the cells described herein can be determined qualitatively and/or quantitatively based on any suitable criterion known in the art, including but not limited to microscopy, cytometry, DNA, mRNA, cDNA, proteins, protein fragments, and/or gene copy number.

TABLE 1
Exemplary M1 macrophage gene signature sets

| Gene | |
|---|---|
| signature | |
| set | Genes |
| 1 | ACP2, ABCD1, C1QA, FDX1, CCL22, CD163, SCAMP2, ADAMDEC1, ARL8B, and HAMP |
| 2 | ACP2, ABCD1, FDX1, CCL8, CCL22, CD163, ADAMDEC1, TREM2, and HAMP |
| 3 | ACP2, ADRA2B, ALCAM, ABCD1, ATOX1, ATP6V0C, ATP6V1E1, BLVRA, C1QA, CD48, |
| | CD63, CLCN7, TPP1, CLTC, CCR1, CMKLR1, SLC31A1, COX5B, FCER1G, FDX1, FOLR2, |
| | FPR3, FTL, HEXB, HK3, IL10, IL12B, ITGAE, LAIR1, CXCL9, MMP19, NARS, NDUFS2, |
| | P2RX7, PDCL, MAPK13, PTGIR, PTPRA, RELA, CCL7, CCL8, CCL19, CCL22, SRC, STX4, |
| | TCEB1, TFRC, AGPS, MARCO, SNX3, CD84, USP14, ITGB1BP1, ATP6V1F, TRIP4, CD163, |
| | CIAO1, WTAP, ARHGEF11, ABI1, SCAMP2, ACTR2, BCAP31, ZMPSTE24, BCKDK, |
| | EXOC5, STIP1, UQCR11, SDS, LILRB4, OGFR, TFEC, FKBP15, DNAJC13, TDRD7, STX12, |
| | IL17RA, ABTB2, FAM32A, SIGLEC7, SIGLEC9, ADAMDEC1, CECR5, SLC25A24, NRBP1, |
| | MS4A4A, TREM2, OTUD4, PQLC2, HAUS2, ARL8B, NECAP2, WDR11, ZC3H15, CCDC47, |
| | UTP3, MRS2, HAMP, MRPL40, VPS33A, CORO7, LIMD2, TMX1, DOT1L, ADO, and ADCK2 |
| 4 | ACP2, ADRA2B, ALCAM, TSPO, C3AR1, DAGLA, CALR, CHIT1, CYBB, CYC1, CYP19A1, |
| | DLAT, FCER1G, GP1BA, GPD1, IFNAR1, IL10, KCNJ5, KIFC3, MT2A, MYBPH, MYH11, |
| | MYO7A, P2RX7, PRDX1, RAB3IL1, RNH1, MRPL12, CCL1, CCL7, CCL8, CCL24, SRC, VIM, |
| | RRP1, MARCO, S1PR2, AP1M2, ACTR3, LILRB1, AFG3L2, SDS, LILRB4, EMILIN1, VSIG4, |
| | HSPB7, COQ2, ADAMDEC1, CECR5, WSB2, SLAMF8, DNASE2B, CLPB, MFSD7, and |
| | ADCK2 |
| 5 | ACP2, ADCY3, ADRA2B, ALCAM, TSPO, C1QA, C1QB, C3AR1, DAGLA, CD63, CHIT1, |
| | CMKLR1, SLC31A1, CSF1, CSF1R, CYBB, CYC1, CYP19A1, FANCE, FCER1G, FDX1, |
| | FPR3, FTL, GP1BA, GPD1, HEXB, IL10, KCNJ1, KCNJ5, KIFC3, LAMP1, MMP19, MSR1, |
| | MT2A, MYBPH, MYO7A, P2RX7, PRDX1, RAB3IL1, MRPL12, CCL1, CCL7, CCL8, CCL18, |
| | CCL19, CCL24, SLC6A12, SPR, SRC, RRP1, MARCO, PKD2L1, S1PR2, CD163, LONP1, |
| | AP1M2, IGSF6, LILRB1, SDS, LILRB4, EMILIN1, VSIG4, TFEC, PHLDB1, CYFIP1, FKBP15, |
| | NCAPH, MYOF, HSPB7, ADAMDEC1, GLRX2, NDUFAF1, SPG21, MS4A4A, ATP6V1D, |
| | ATP6V1H, TREM2, PQLC2, TMEM70, PLEKHB2, TMEM33, SLAMF8, HAMP, DNASE2B, |
| | MYOZ1, LONRF3, CLPB, MFSD7, and ADCK2 |
| 6 | ACP2, ADCY3, ADRA2B, ALCAM, ABCD1, ANXA2, ATP6V1A, C1QA, C1QB, C3AR1, |
| | DAGLA, CD80, CD63, CHIT1, CMKLR1, SLC31A1, CSF1, CSF1R, CYBB, CYC1, CYP19A1, |
| | FANCE, FDX1, FPR2, FPR3, GPD1, HEXB, KCNJ1, KCNJ5, KIFC3, MMP19, MSR1, MT2A, |
| | MYBPH, P2RX7, MAPK13, S100A11, CCL1, CCL7, CCL8, CCL18, CCL19, CCL22, CCL24, |
| | SLC1A2, SLC6A12, SLC11A1, SIGLEC1, SRC, TIE1, MARCO, HYAL2, CD163, LONP1, |
| | IGSF6, LILRB1, CD300C, SDS, LILRB4, EMILIN1, VSIG4, PHLDB1, NCAPH, CLEC4E, |
| | MYOF, HSPB7, ADAMDEC1, GLRX2, MS4A4A, ATP6V1H, TREM2, TMEM70, TMEM33, |
| | KCNK13, SLAMF8, HAMP, DNASE2B, MYOZ1, MFSD7, ADO, ADCK2, and TBC1D16 |

TABLE 2
Exemplary macrophage gene signature sets

| Gene | |
|---|---|
| signature | |
| set | Genes |
| 1 | ACP2, ATOX1, ATP6V0C, ATP6V1E1, C1QA, CD9, CD48, CLCN7, CCR1, CRYBB1, CYBB, |
| | FCER1G, FDX1, FOLR2, FPR3, FTL, HEXA, HEXB, KCNJ5, LAIR1, M6PR, MDH1, NUBP1, |
| | P2RX7, RAC1, CCL8, CCL22, STX4, TCEB1, TYROBP, VAMP8, MARCO, CD84, |
| | ATP6V0E1, ATP6V1F, CD163, CIAO1, LY86, ARHGEF11, HS3ST2, ARPC4, ATP6AP2, |
| | BAIAP2, PRDX3, ERP29, LILRB5, SDS, LILRB4, VSIG4, FKBP15, ZZZ3, SIGLEC7, |
| | SIGLEC9, ADAMDEC1, ORMDL2, COMMD9, SPG21, MS4A4A, UBE2D4, TRAPPC2L, |
| | TREM2, TMEM70, ARL8B, TMEM126B, SLAMF8, C12orf4, HAMP, GUF1, and YIF1B |
| 2 | ACP2, ATOX1, ATP6V0C, ATP6V1E1, C1QA, CD9, CD48, CD63, CLCN7, TPP1, CCR1, |
| | CMKLR1, SLC31A1, COX5B, COX7B, COX8A, CRYBB1, CYBA, CYBB, FCER1G, FDX1, |
| | FOLR2, FPR3, FTL, GLB1, HEXA, HEXB, KCNJ5, LAIR1, LAMP1, NUBP1, NDUFB3, |
| | NDUFS3, NDUFS6, P2RX7, PCMT1, MAPK13, PSME1, RB1, CCL8, CCL22, SDHD, SUMO3, |
| | SNX2, TCEB1, TYROBP, UQCRC2, VAMP8, MARCO, SNX3, CD84, ATP6V0E1, ITGB1BP1, |
| | S1PR2, ATP6V1F, CD163, COX5A, CIAO1, LY86, ARHGEF11, HS3ST2, SCAMP2, ARPC4, |
| | ATP6AP2, BAIAP2, PRDX3, ERP29, UQCR11, LILRB5, SDS, LILRB4, VSIG4, STAB1, |
| | FKBP15, DNAJC13, CLEC5A, ZZZ3, SIGLEC7, SIGLEC9, ADAMDEC1, ORMDL2, |
| | COMMD9, UQCR10, SPG21, MS4A4A, UBE2D4, TRAPPC2L, TREM2, KCTD5, PQLC2, |
| | COMMD8, TMEM70, ARL8B, SLC38A7, NOP10, WDR11, TMEM126B, TMEM9B, SLAMF8, |
| | C12orf4, MRS2, HAMP, DNASE2B, GUF1, MS4A6A, MRPL40, PPCS, PMFBP1, YIF1B, |
| | ADCK2, and HIGD2A |
| 3 | ACP2, ATOX1, ATP6V0C, ATP6V1E1, C1QA, CD9, CD48, CD63, CLCN7, CCR1, COX8A, |
| | CRYBB1, CYBB, FCER1G, FDX1, FOLR2, FPR3, FTL, HEXA, HEXB, KCNJ5, LAIR1, |
| | LAMP1, NDUFB3, NDUFS6, P2RX7, MAPK13, CCL8, CCL22, SNX2, TCEB1, TYROBP, |
| | UQCRC2, VAMP8, MARCO, SNX3, CD84, ATP6V0E1, ITGB1BP1, ATP6V1F, CD163, |
| | COX5A, CIAO1, LY86, ARHGEF11, HS3ST2, SCAMP2, ARPC4, ATP6AP2, ERP29, |
| | UQCR11, LILRB5, SDS, LILRB4, VSIG4, STAB1, FKBP15, DNAJC13, CLEC5A, ZZZ3, |
| | SIGLEC7, SIGLEC9, ADAMDEC1, ORMDL2, COMMD9, SPG21, MS4A4A, UBE2D4, |
| | TREM2, KCTD5, TMEM70, ARL8B, NOP10, WDR11, TMEM126B, SLAMF8, C12orf4, HAMP, |
| | GUF1, MS4A6A, MRPL40, PPCS, PMFBP1, YIF1B, and ADCK2 |
| 4 | ACP2, CHIT1, CSF1, CYP19A1, FDX1, HK3, MSR1, CCL22, SLC6A12, CD84, SDS, VSIG4, |
| | CLEC5A, ADAMDEC1, HAMP, DNASE2B, and MYOZ1 |
| 5 | ACP2, ADCY3, ALCAM, ABCD1, BPI, CHIT1, CYP19A1, HK3, KCNJ1, KCNMB1, CXCL9, |
| | MMP8, MSR1, CCL1, CCL7, CCL22, SLC1A2, SLC6A12, CD84, CD163, SDS, LILRB4, |
| | VSIG4, FKBP15, NCAPH, CLEC5A, ADAMDEC1, ATP6V1H, PQLC2, SLAMF8, HAMP, |
| | DNASE2B, MYOZ1, LONRF3, and MFSD7 |
| 6 | ACP2, ADCY3, ALCAM, ABCD1, ATP6V1A, ATP6V0A1, BPI, CHIT1, SLC31A1, CSF1, |
| | CYBB, CYP19A1, FDX1, FGR, HK3, ITGAX, KCNJ1, KCNMB1, CXCL9, MMP8, MMP19, |
| | MSR1, P2RX7, MAPK13, CCL1, CCL7, CCL22, SLC1A2, SLC6A12, SLC11A1, STX4, NUMB, |
| | MARCO, CD164, CD84, CD163, CIR1, ARHGEF11, BCAP31, ATP6AP2, SDS, LILRB4, |
| | VSIG4, FKBP15, NCAPH, CLEC5A, ADAMDEC1, ATP6V1H, PQLC2, CCDC88A, PCDHB11, |
| | SLAMF8, HAMP, DNASE2B, MYOZ1, LONRF3, and MFSD7 |
| 7 | ACADVL, ACP2, ARSB, ATP6V1A, ATP6V0C, ATP6V1C1, CD63, CETN2, CCR1, SLC31A1, |
| | COX5B, COX15, CYBB, DBI, ECHS1, FDX1, GRB2, HADHB, HEXA, HEXB, HK3, HMGCL, |
| | ITGAX, KIFC3, TNPO1, LAIR1, LAMP1, MGST3, MSR1, NARS, NDUFA8, NDUFB6, |
| | NDUFS8, PRDX1, PEX14, MAPK13, PSMD10, PTPN12, PEX19, QDPR, RAB1A, RAB5C, |
| | RALA, RENBP, CLIP1, CCL7, CCL18, SDHB, SRC, STX4, TCEB1, MLX, TRAF3, NSMAF, |
| | AGPS, MARCO, SNX4, ATP6V1F, LONP1, GSTO1, BAG3, CIR1, FEZ2, PDCD6IP, SNUPN, |
| | BCAP31, STAM2, IGSF6, ZMPSTE24, TMEM147, VTI1B, TGOLN2, SPIN1, LILRB4, |
| | EMILIN1, VSIG4, EFR3A, FKBP15, CLEC5A, IBTK, NPTN, ATP2C1, SIGLEC9, ADAMDEC1, |
| | CNIH4, GLRX2, DERA, NDUFAF1, HSD17B12, ZDHHC3, TNFRSF12A, MS4A4A, ATP6V1D, |
| | ATP6V1H, TMBIM4, STYXL1, BTBD1, NUDT9, TMEM33, NOP10, TMEM127, ACTR10, |
| | KCMF1, TULP4, C12orf4, RTN4, MKL2, HAMP, DNASE2B, KLHL12, NSUN3, ELOVL1, |
| | SLC30A5, MAPKAP1, LONRF3, TCEAL4, CHD9, TM2D1, MFSD7, G6PC3, TBC1D16, and |
| | ZDHHC24 |
| 8 | ACP2, ARSB, ATP6V1A, ATP6V0C, ATP6V1C1, CD63, CETN2, CCR1, SLC31A1, COX5B, |
| | COX15, CYBB, DBI, ECHS1, FDX1, HEXB, LAIR1, LAMP1, MGST3, MSR1, NDUFA8, |
| | NDUFS8, PRDX1, MAPK13, PSMD10, PTPN12, PEX19, RAB1A, RALA, RENBP, CCL7, |
| | SDHB, STX4, TCEB1, MLX, TRAF3, NSMAF, AGPS, MARCO, ATP6V1F, GSTO1, BCAP31, |
| | IGSF6, TMEM147, VTI1B, LILRB4, EMILIN1, VSIG4, EFR3A, CLEC5A, NPTN, ATP2C1, |
| | SIGLEC9, ADAMDEC1, CNIH4, GLRX2, DERA, NDUFAF1, ZDHHC3, TNFRSF12A, |
| | MS4A4A, ATP6V1D, ATP6V1H, NUDT9, TMEM33, C12orf4, RTN4, HAMP, DNASE2B, |
| | ELOVL1, SLC30A5, MAPKAP1, LONRF3, MFSD7, G6PC3, TBC1D16, and ZDHHC24 |
| 9 | ACADVL, ACP2, ARSB, ATP6V1A, ATP6V0C, ATP6V1C1, CD63, CETN2, CCR1, SLC31A1, |
| | COX5B, COX15, CYBB, DBI, ECHS1, FDX1, GRB2, HADHB, HCCS, HEXA, HEXB, HK3, |
| | HMGCL, ITGAX, TNPO1, LAIR1, LAMP1, MGST3, MSR1, NDUFA8, NDUFS8, PRDX1, |
| | MAPK13, PSMD10, PTPN12, PEX19, QDPR, RAB1A, RALA, RENBP, CLIP1, CCL7, SDHB, |
| | SRC, STX4, TCEB1, MLX, TRAF3, UQCRC2, USF2, NSMAF, AGPS, MARCO, SNX4, |
| | ATP6V1F, GSTO1, PDCD6IP, BCAP31, STAM2, IGSF6, ZMPSTE24, CEPT1, TMEM147, |
| | VTI1B, TGOLN2, SPIN1, LILRB4, TMEM115, EMILIN1, VSIG4, EFR3A, FKBP15, CLEC5A, |
| | NPTN, ATP2C1, TRAPPC3, SIGLEC9, ADAMDEC1, CNIH4, SLC25A24, GLRX2, DERA, |
| | NDUFAF1, ZDHHC3, TNFRSF12A, MS4A4A, ATP6V1D, ATP6V1H, BTBD1, NUDT9, |
| | TMEM33, TMEM127, ACTR10, KCMF1, TULP4, C12orf4, RTN4, MKL2, HAMP, DNASE2B, |
| | NSUN3, ELOVL1, SLC30A5, C7orf25, MAPKAP1, PANK3, LONRF3, TM2D1, MFSD7, |
| | SETD3, G6PC3, TBC1D16, and ZDHHC24 |
| 10 | ACP2, ADCY3, ABCD1, ATP6V1A, CHIT1, CLCN7, TPP1, SLC31A1, COX5B, CYP19A1, |
| | FDX1, HEXA, HK3, KCNJ1, M6PR, MSR1, SLC6A12, CD164, CD84, LONP1, ATP6AP2, |
| | TGOLN2, SDS, LILRB4, VSIG4, FKBP15, CLEC5A, ADAMDEC1, ATP6V1H, STX18, PQLC2, |
| | DNASE2B, MYOZ1, SLC30A5, LONRF3, MFSD7, and LDHAL6B |
| 11 | ACP2, ADCY3, ALCAM, ABCD1, ARSB, ATOX1, ATP6V1A, ATP6V0C, ATP6V1E1, |
| | ATP6V0A1, BPI, TSPO, CD63, CHIT1, CLCN7, TPP1, SLC31A1, COX5B, CSF1, CYBB, |
| | CYP19A1, FDX1, HTT, HEXA, HK3, ITGAX, KCNJ1, LAMP1, M6PR, MMP19, MSR1, |
| | MTHFR, P2RX7, MAPK13, PTPRA, RABGGTA, SLC1A2, SLC6A12, STX4, TCEB1, NUMB, |
| | CD164, CD84, LONP1, CPNE6, CIR1, TTLL4, ARHGEF11, BCAP31, ATP6AP2, TGOLN2, |
| | SDS, LILRB4, VSIG4, FKBP15, CLEC5A, IL17RA, ADAMDEC1, NRBP1, SH3GLB1, |
| | ATP6V1H, STX18, PQLC2, TMEM33, SLC38A7, VPS53, CCDC88A, SLAMF8, HAMP, |
| | DNASE2B, MYOZ1, MTMR14, SLC30A5, MUL1, C12orf49, LONRF3, MFSD7, LDHAL6B, and |
| | ZDHHC24 |
| 12 | ACP2, CHIT1, CSF1, CYP19A1, FDX1, HK3, MSR1, CCL22, SLC6A12, CD84, SDS, VSIG4, |
| | CLEC5A, ADAMDEC1, HAMP, DNASE2B, and MYOZ1 |
| 13 | APOE, ATG7, BCAT1, CCL7, CD163, CD68, CD84, CHI3L1, CHIT1, CLEC5A, COL8A2, |
| | COLEC12, CTSK, CXCL5, CYBB, DNASE2B, EMP1, FDX1, FN1, GM2A, GPC4, KAL1, |
| | MARCO, ME1, MS4A4A, MSR1, PCOLCE2, PTGDS, RAI14, SCARB2, SCG5, SGMS1, and |
| | SULT1C2 |
| 14 | AIF1, CCL1, CCL14, CCL23, CCL26, CD300LB, CNR1, CNR2, EIF1, EIF4A1, FPR1, FPR2, |
| | FRAT2, GPR27, GPR77, RNASE2, MS4A2, BASP1, IGSF6, HK3, VNN1, FES, NPL, FZD2, |
| | FAM198B, HNMT, SLC15A3, CD4, TXNDC3, FRMD4A, CRYBB1, HRH1, and WNT5B |
| 15 | FUCA1, MMP9, LGMN, HS3ST2, TM4SF19, CLEC5A, GPNMB, C11orf45, CD68, and CYBB |
| 16 | CD163, CD14, CSF1R, C1QC, VSIG4, C1QA, FCER1G, F13A1, TYROBP, MSR1, C1QB, |
| | MS4A4A, FPR1, S100A9, IGSF6, LILRB4, FPR3, SIGLEC1, LILRA1, LYZ, HK3, SLC11A1, |
| | CSF3R, CD300E, PILRA, FCGR3A, AIF1, SIGLEC9, FCGR1C, OLR1, TLR2, LILRB2, |
| | C5AR1, FCGR1A, MS4A6A, C3AR1, HCK, IL4I1, LST1, LILRA5, CSTA, IFI30, CD68, |
| | TBXAS1, FCGR1B, LILRA6, CXCL16, NCF2, RAB20, MS4A7, NLRP3, LRRC25, ADAP2, |
| | SPP1, CCR1, TNFSF13, RASSF4, SERPINA1, MAFB, IL18, FGL2, SIRPB1, CLEC4A, |
| | MNDA, FCGR2A, CLEC7A, SLAMF8, SLC7A7, ITGAX, BCL2A1, PLAUR, SLCO2B1, PLBD1, |
| | APOC1, RNF144B, SLC31A2, PTAFR, NINJ1, ITGAM, CPVL, PLIN2, C1orf162, FTL, LIPA, |
| | CD86, GLUL, FGR, GK, TYMP, GPX1, NPL, and ACSL1 |

TABLE 3
Exemplary 153-gene signature matrix

| | | M1 | M2 | | |
|---|---|---|---|---|---|
| Gene | B cells | Macrophages | Macrophages | Monocytes | Neutrophils |
| ABCB4 | 22.7138212901797 | 2.23179950051687 | 1.97155445562159 | 0.105994738308192 | 0 |
| ADAM28 | 138.122317697073 | 44.549173431875 | 4.86154374716257 | 6.15419538796811 | 6.92361545168478 |
| ADAM6 | 311.524313608549 | 0.185500319951345 | 0.387193475322115 | 0.0309434181627899 | 0.0313699237303561 |
| AFF3 | 74.3798680534155 | 1.41947594130957 | 1.79918141862698 | 2.90259697885655 | 1.79219821564678 |
| AKAP2 | 90.5380895821784 | 17.5227274563533 | 3.32632182691764 | 0.221090667019584 | 0 |
| ARHGAP24 | 48.923113469842 | 10.4272519545846 | 2.52236669354933 | 17.6398417143727 | 3.12847520545414 |
| BANK1 | 301.24413442937 | 4.00037342190056 | 7.12162949184432 | 2.43007530175162 | 0.0467090734061819 |
| BCL11A | 162.35746258436 | 2.02454979826139 | 9.5209120731403 | 21.7927458929775 | 1.74970688550885 |
| BLK | 529.702738900353 | 0.804533642108712 | 0.725478229136685 | 0.162953576261731 | 0.112842610698866 |
| BLNK | 174.401225832058 | 2.41629459859149 | 61.5054350055487 | 1.56419570294049 | 0.0488792691033357 |
| CD19 | 439.268676213828 | 0.940667995897954 | 0.960748363506361 | 0.339789686883238 | 0.105206787085179 |
| CD200 | 55.2294664656558 | 0 | 1.41548056084562 | 0 | 0.662093077794551 |
| CD72 | 258.499972469939 | 12.484112301443 | 4.9256997230085 | 3.4455066344001 | 0.150684735222654 |
| EBF1 | 33.9402843306255 | 0.0419617982650429 | 0.107810010150852 | 0.0258455531259009 | 0.0263452108172489 |
| FCMR | 560.208427058502 | 4.33144930960849 | 1.48636123601938 | 0.38458372366092 | 11.0654267178681 |
| FCRLA | 240.512072319375 | 1.50019097388064 | 0.619542712060854 | 0.040850234118554 | 0 |
| GNG7 | 120.485122831339 | 3.36311354485396 | 5.80324654779615 | 4.72007086693286 | 13.7628682574527 |
| HLA-DOB | 372.459806363368 | 7.07984181889178 | 3.25821368357222 | 9.5537257936507 | 15.7027822745319 |
| KLHL14 | 37.7430063578197 | 0 | 0 | 0 | 0 |
| MEF2C | 116.323311328885 | 5.89045977701664 | 11.1354921599087 | 31.5715802571576 | 7.85772776063656 |
| P2RX5 | 635.399774973654 | 1.81920580725339 | 1.53569999555268 | 5.36565883803721 | 4.20649120944489 |
| PCDH9 | 25.1154006032258 | 0.0471608297055587 | 0.0346111724453279 | 0.0135109763504724 | 0 |
| PNOC | 102.303071324944 | 0.481037399096269 | 0.221787282606074 | 3.674790248062 | 0 |
| POU2AF1 | 210.360922846279 | 1.07402574055616 | 1.02531762861916 | 1.25038125930661 | 1.42186066661587 |
| RALGPS2 | 243.443027497961 | 5.06750774949951 | 14.4045255072051 | 4.23115886707842 | 11.6209486813389 |
| SPIB | 224.463150722433 | 0.482360222996223 | 0.70036408031993 | 0.141625346838359 | 0.0518068448663745 |
| STAP1 | 138.998205062899 | 0.75443517176183 | 1.78548158989623 | 0.137633946325403 | 0.174061069300259 |
| TCF4 | 66.326842586527 | 3.68019267419876 | 26.7980773610538 | 5.19195703557171 | 0.198946422341884 |
| TCL1A | 551.872721659835 | 0 | 0 | 0 | 0.0461283203003378 |
| VPREB3 | 288.520510513099 | 0.494539146107356 | 0 | 0 | 0 |
| ACVRL1 | 0.332585294715274 | 19.7772120444697 | 2.10520049097964 | 1.58765673970166 | 0.424882227546612 |
| ADAMDEC1 | 0.635314747578684 | 183.705448066853 | 9.49282348979648 | 1.20280845755159 | 0.054395364353221 |
| ALDH1A2 | 0 | 23.1067632472012 | 9.7981847931226 | 0.123003597462278 | 0.571071038124148 |
| CCRL2 | 1.49069074245513 | 68.3022602497262 | 8.21384716771491 | 14.8175992642263 | 3.39577878674019 |
| CD86 | 14.8419681265522 | 415.559752234066 | 141.598162873142 | 124.373970474268 | 0.0260916172816413 |
| CFB | 0.152608724773573 | 65.9967893726307 | 17.9938750647176 | 1.51149883576967 | 0.231942366645218 |
| CXCL2 | 0.109297174031566 | 302.903768620453 | 34.1076360418172 | 47.1810943911007 | 2.43196954555697 |
| CXCL3 | 0 | 259.154144878705 | 51.7012610500732 | 6.61824121804823 | 0.422991300148712 |
| EREG | 1.426795625176 | 163.050043614166 | 15.6097902146948 | 23.7225799821433 | 1.39213256974851 |
| ICAM1 | 6.17602138151068 | 592.921253202952 | 207.459056399526 | 125.338706945171 | 121.463688878235 |
| INHBA | 0.0639257465975131 | 578.149564143944 | 21.2499318026688 | 0.203242228223779 | 0.345224461315037 |
| LILRB4 | 3.78654567485434 | 476.82255473671 | 133.464950198138 | 124.041784829028 | 18.5815069394489 |
| NUPR1 | 0 | 101.322179479238 | 22.4329877889438 | 0.0882116265122513 | 0.0811838909277743 |
| PDPN | 0.263864646113126 | 16.776474481883 | 6.31494558426671 | 0.370034834512035 | 1.56290059271976 |
| SERPING1 | 0 | 500.333873998758 | 17.3112787031335 | 27.543535044725 | 77.6732315399919 |
| SLC1A3 | 0.0356287531145159 | 25.7591310157418 | 5.95100851102093 | 0.927036634840135 | 0.0340795710863373 |
| SLC6A12 | 0 | 40.8791807642813 | 1.66954056123124 | 6.85529805658659 | 3.24619240517486 |
| SQOR | 26.5317324389891 | 535.536312247838 | 177.585739894353 | 126.37905571682 | 191.98901026902 |
| VSIG4 | 0.109900894382725 | 287.034988195206 | 8.91664562329146 | 8.59675031145639 | 1.20698712377612 |
| ACE | 3.7992101423842 | 92.2488019058023 | 303.464970506693 | 2.53539965563725 | 3.34883479329738 |
| CISH | 0.737194776103433 | 103.895600398754 | 239.503450756414 | 4.85971934064406 | 8.5357285604466 |
| CLEC4A | 10.4771933509048 | 71.6302696769377 | 400.766557876869 | 149.530636617981 | 135.985920646415 |
| CLIC2 | 0.694483626998776 | 30.7996880352197 | 133.692558026865 | 5.06218812509425 | 2.25505284322546 |
| COLEC12 | 0 | 23.2936430965653 | 78.8953739723945 | 1.16683303498152 | 0 |
| ECM1 | 1.16224794525222 | 25.2617977050096 | 51.7673538087741 | 0.851277964741539 | 0.125565820900312 |
| ENPP2 | 0.798089326413484 | 23.2877128002904 | 59.124417778249 | 1.00560930051376 | 0.241410922855034 |
| GATM | 6.24630433858738 | 1.30201783504023 | 113.238809577875 | 1.02984423835886 | 0 |
| GFRA2 | 0.130888080063566 | 4.83084435375596 | 79.2607177166156 | 2.06724773612502 | 0 |
| IL1R1 | 0.51108490301588 | 30.803556063969 | 212.912935199361 | 2.3804779784133 | 25.4042245502966 |
| MAOA | 0 | 5.95645516617961 | 80.1465476825236 | 0.103115807610358 | 0 |
| MS4A4A | 0.13127327186729 | 30.3792176392599 | 212.758993973887 | 12.9864959847751 | 0 |
| PLTP | 4.09773209191575 | 15.5762699043529 | 90.072255489694 | 2.13834344792328 | 0.631419406467024 |
| PTGS1 | 17.7102769818648 | 23.5081781658257 | 111.231364417806 | 26.2406466909419 | 5.36821745232743 |
| RNASE6 | 64.1140064772035 | 206.972276626839 | 633.832133210133 | 201.794703201243 | 45.1798115742456 |
| SLC7A8 | 0.237455366576669 | 12.0565466071266 | 87.1386494103188 | 0.292623094712473 | 0.0562275499429652 |
| TREM2 | 0.0436228969244222 | 25.0828674728233 | 204.084827382242 | 0.719506568957995 | 0.330206010339332 |
| WNT5B | 0.124075186063689 | 0.373209145686068 | 21.0485792500978 | 1.53606464649534 | 0.0285119129727064 |
| CD1D | 22.9579813644177 | 3.34560477744368 | 1.12160518738656 | 109.71255533243 | 4.25317725185491 |
| FXYD6 | 0.0499479108396515 | 8.14251465517473 | 1.51808441059495 | 51.1474894647036 | 0.163029810701658 |
| GAS7 | 3.05073991381932 | 38.413665207398 | 35.0489843958423 | 109.921417466546 | 32.3679507043919 |
| HPSE | 1.17369291780026 | 2.9430035890169 | 2.29581082691957 | 77.9788201672732 | 35.4940239786558 |
| KBTBD11 | 0.384689196355843 | 4.84433108833272 | 6.5406673151378 | 11.5850984842579 | 4.85401131707557 |
| NRG1 | 0 | 0 | 1.01807919863294 | 35.4297965389362 | 0 |
| SMARCD3 | 4.59071900115077 | 15.6068632596657 | 10.643282677309 | 152.811686007079 | 23.7139348285711 |
| TMEM170B | 2.45693897803563 | 24.6567425931697 | 8.88862444506901 | 77.1849568226254 | 10.9176069559463 |
| ABHD5 | 2.4032915401641 | 30.1571858535619 | 13.1805019532929 | 18.5733113292649 | 68.0693895185095 |
| ADGRG3 | 0.131466148745403 | 0.472304260413813 | 0.152058295813748 | 0.538263293930684 | 571.897708115885 |
| BTNL8 | 0.368149861441642 | 0.302261559138706 | 0 | 0.800757200154997 | 104.95658070008 |
| CEACAM1 | 6.87952133740813 | 1.94479031483905 | 1.36810402032269 | 2.96191872941251 | 74.5043059312033 |
| DAPK2 | 5.07134007453929 | 1.13104439724161 | 0.816545662777064 | 2.23079479124051 | 100.413351085799 |
| DGAT2 | 3.82158723795715 | 7.67644326641274 | 6.2186171673132 | 6.58444059177283 | 327.49380508872 |
| DPEP3 | 2.59260003814738 | 0.533960079085381 | 1.93834876262687 | 1.66611711910439 | 34.8086167483078 |
| DYSF | 0.141021184240563 | 14.9194244156264 | 7.23975607048224 | 43.5038034974759 | 333.701448218021 |
| FPR2 | 0.0888493853574401 | 41.6179915042076 | 4.47574697178295 | 39.2481280641213 | 669.095997076219 |
| KCNJ15 | 0.034450621804724 | 20.9510319819367 | 1.07629254998603 | 3.98484698454836 | 361.827478544255 |
| LILRA2 | 0.947022274267314 | 90.3028375625655 | 8.07042991836322 | 288.430056964502 | 607.112046566405 |
| LRRC4 | 0.617728719549034 | 0.214277039161762 | 1.38891714902995 | 3.78613786610382 | 85.9786297945884 |
| MGAM | 0.0471277210657418 | 8.85197804080068 | 0.407997709201937 | 3.37314275253331 | 319.245026714378 |
| MME | 1.29490791514107 | 0.558026968296873 | 0.170019176920162 | 0.538787242608576 | 382.109462967198 |
| REM2 | 12.3258997018747 | 1.75338875771919 | 2.47587455119383 | 1.27872108996671 | 81.4152262930432 |
| REPS2 | 2.24282263338729 | 0.954439466148988 | 10.1087744474924 | 3.43139437886716 | 32.7349262478794 |
| TMCC3 | 6.57322429545358 | 8.14793681989921 | 6.12198592655159 | 6.33101986919401 | 59.9283524527154 |
| ABCB1 | 4.63154686733255 | 0.715223790192447 | 0.287480957998641 | 0 | 0 |
| AUTS2 | 10.4292142934928 | 0.378491284950299 | 0.70563506554458 | 0.350534093801845 | 0.0468427807798921 |
| COLGALT2 | 0.0503339008113001 | 0.691005435055901 | 2.83892801522651 | 0.0427929381517172 | 0.0235958819004023 |
| EOMES | 0.0294414936808075 | 0 | 0 | 0 | 0 |
| FASLG | 0.406052635272513 | 0.152148329940168 | 0.0619085809277628 | 0.354963620298727 | 0.165275606590322 |
| GK5 | 10.4441946202148 | 13.9822172169163 | 8.68370624717098 | 10.6140157840911 | 2.65354163119785 |
| GNG2 | 39.8257313163402 | 4.58086240903197 | 16.6046687273083 | 52.8962086615344 | 65.4887065821961 |
| HOPX | 17.2321146202866 | 3.16965457911556 | 1.47623189663556 | 0.131760621540902 | 0.235345152176187 |
| KLRD1 | 2.1730920691707 | 1.33460636734408 | 1.72022815373913 | 1.66168543626761 | 1.648031188641 |
| MATK | 5.0697215317486 | 65.4922061613533 | 45.4480692719509 | 0.304760770589686 | 0.192243835097329 |
| MLC1 | 0.0485219973466009 | 0.0641268697938566 | 0.538831709037623 | 2.68953562519948 | 0.264360540457374 |
| NCALD | 0.0436602836343503 | 0.0742748478569402 | 0.0289257130114139 | 0.0327413298290547 | 0.0158724306087475 |
| NLRC3 | 41.0377217209724 | 1.30118117064042 | 0.760087584031309 | 0.724039617208233 | 2.34517996463339 |
| PDGFD | 0.235737723867995 | 0.0381807935343397 | 0.0807138003344366 | 0 | 0 |
| PLEKHF1 | 2.89906019044632 | 5.57735836943224 | 1.31936324244112 | 0.155325651351853 | 0.0677775107833816 |
| PPP2R2B | 0.0562970804183874 | 0 | 1.00444928865373 | 0.0983155589802625 | 0.121169066030033 |
| PYHIN1 | 10.8375867804319 | 1.13507144800805 | 0.481226732421874 | 0.172259027619941 | 0.672550480103105 |
| RNF165 | 0 | 0 | 0.0219641170576724 | 0.048744954610262 | 0.0243795407887355 |
| S1PR5 | 0.0618605571367731 | 0.074065425890921 | 0.312335075546808 | 0.100674680009297 | 0.0285574435648188 |
| SAMD3 | 3.15766899105223 | 0.768173205038007 | 0.423872667811168 | 0.730838797177124 | 0.451777467510685 |
| SBK1 | 0.89683364209223 | 0.244026650940044 | 0.107697845591846 | 0.273248007380871 | 0.313675097000316 |
| SH2D2A | 0.417387642431526 | 0.399430552086268 | 1.0692672878798 | 0 | 0.0869516539121953 |
| SLFN13 | 4.93323201574845 | 2.98630116225055 | 3.19026545673749 | 0.968729048124671 | 1.22170364547377 |
| STAT4 | 3.5137613091855 | 11.4359609879083 | 6.86205037725543 | 1.31034705378228 | 1.68724203860777 |
| TOGARAM2 | 0.14977604730489 | 0.682597729197908 | 1.38778238309787 | 1.48423663565902 | 0.275934616545652 |
| TSPOAP1 | 5.13086518690353 | 5.07246402179412 | 2.13312627831793 | 11.4858669883818 | 3.0399898258509 |
| TTC38 | 3.85182444236592 | 9.26844279463642 | 13.4183090394676 | 14.3877671325781 | 4.57582303947228 |
| TXK | 1.67435589560267 | 1.7969541080826 | 1.81590794963782 | 1.27335372931424 | 1.18412287042393 |
| YPEL1 | 23.9202438542052 | 1.75716237532657 | 7.6419948072116 | 1.71145377856093 | 1.90757394112732 |
| ZBTB16 | 14.2955085383565 | 0.562626343097506 | 0.535758215179425 | 8.474397823996 | 1.24492386029471 |
| FHIT | 3.33523058079424 | 4.08008791045851 | 5.79890885711685 | 3.61138210623977 | 0.31545962658046 |
| TRAT1 | 0.0642279284285004 | 0.175857133819589 | 0.643153242744908 | 0 | 0 |
| TSHZ2 | 0.282395365806985 | 0.0694927864465123 | 0.0737068227574146 | 0.0143946971064045 | 0.28327099666231 |
| TSPAN18 | 1.77311089111071 | 0.224973142556574 | 0.299670125973253 | 0.303297482065292 | 0.282454969067344 |
| WNT7A | 0 | 0 | 0 | 0 | 0 |
| CD8A | 0.123493752001712 | 0.611395299403943 | 0.53185577970834 | 0.0776055292528393 | 0.104437399218878 |
| CD8B | 2.29233359395268 | 1.80448168335034 | 3.84684462269583 | 1.28711293298135 | 2.29857071513904 |
| FCGBP | 4.19498815568179 | 0.0449441940887562 | 0.0571257486083144 | 0.0989068734123658 | 0.00501899302867541 |
| CCR4 | 0.127506702116757 | 0 | 0.30773593977729 | 0 | 0 |
| CD5 | 4.87737029057527 | 2.23528835363798 | 1.64234966363638 | 0.453178382198983 | 1.14838938173163 |
| CTLA4 | 0 | 0.939719253999237 | 1.14540488404531 | 0.0845014947257681 | 0 |
| FAM160B1 | 11.6727963252047 | 16.3066269771222 | 18.7870913232553 | 11.5574787297536 | 35.6428461439641 |
| FAM46C | 116.520091293196 | 0.786458366097308 | 1.02949567580101 | 0.121436258116005 | 3.58577926298475 |
| FOXP3 | 0.966707150988455 | 0.572910937281857 | 0.82573435848077 | 0.599754930500575 | 0 |
| GCNT4 | 0 | 0.128566977556141 | 0.0941949576915401 | 0.0129487954235771 | 0 |
| HPGD | 0.297346138478579 | 2.40675869902318 | 0.423144116281786 | 0.437543775657933 | 1.26252571799256 |
| ICOS | 0 | 0.293128153030223 | 0.256265337095090 | 0.0176882892358036 | 0 |
| LAYN | 0 | 0.0835626075128009 | 0.587878540672166 | 0 | 0 |
| LRRC32 | 1.92104533534765 | 10.2017663613868 | 0.837196973744059 | 0.599752724112324 | 0.0416687651863303 |
| PLCL1 | 0.104985030369203 | 2.44685686557505 | 5.08020913098262 | 1.62446678236619 | 2.16952878835384 |
| RTKN2 | 3.57035800815153 | 1.00177009348764 | 1.12522533615193 | 1.77421111129307 | 2.11788949245385 |
| SEMA3G | 0.0389765740960512 | 0.577584859041355 | 1.07254018565268 | 0.0354487377226781 | 0 |
| AATBC | 0.446909421795834 | 1.54300657250473 | 4.90994279344639 | 27.1038477887334 | 0.239048944286304 |
| FZD1 | 0.388966762996538 | 13.7784554207995 | 14.8284535753208 | 14.0384856679828 | 0.0469713753506456 |
| GRK3 | 45.9327801347347 | 49.0179285100589 | 66.8058033822207 | 61.5685769015622 | 3.4363986793443 |
| GUCY1A3 | 0.568909387506678 | 0.191756590854844 | 0 | 0.404019093331346 | 0.0148675306736854 |
| KDM1B | 14.9453686150132 | 59.9568838934326 | 46.529497052511 | 64.3965304809244 | 38.7289987387649 |
| LMO2 | 12.1479226527841 | 65.7025664762441 | 116.938733709168 | 108.097739911285 | 73.589743363124 |
| RNF144B | 7.19277800956162 | 40.058627103598 | 32.4435389643522 | 36.9045827477536 | 52.946478435322 |
| SLC2A6 | 62.219054604372 | 37.1231330527007 | 24.7683926116702 | 99.00556540819 | 1.27878370982374 |
| TTYH2 | 2.12221597740415 | 6.61923959128669 | 22.4669511772236 | 43.3162169610057 | 0.910160868995527 |

| | NK | CD4 T | CD8 T | | Dendritic |
|---|---|---|---|---|---|
| Gene | cells | cells | cells | Tregs | cells |
| ABCB4 | 0.236595831817282 | 0.0419535587353655 | 0.107002104922173 | 0.046410247442641 | 2.14790312131434 |
| ADAM28 | 16.0381494634342 | 0.0848609886518498 | 0.0793306796491496 | 0.662666725813814 | 18.9158947025691 |
| ADAM6 | 0.501235722794415 | 0.480615237724707 | 0.390699771827651 | 0.31171570243624 | 0.0777842223142737 |
| AFF3 | 5.31305820583621 | 3.33874242925966 | 1.6719953555597 | 3.41073423545352 | 9.31677002188692 |
| AKAP2 | 6.84541044568527 | 5.8905594487714 | 1.27121634621691 | 18.3633991365087 | 3.30470201093971 |
| ARHGAP24 | 0.052504487033627 | 0.0106536459980673 | 0 | 0.21506860740811 | 2.34976503501111 |
| BANK1 | 1.14720902003051 | 0.0516732285128874 | 0.158108993406954 | 0.0695917288799809 | 4.13538374563377 |
| BCL11A | 0.328324746898523 | 0.333422102683569 | 0.306429474503156 | 0.129487166590795 | 24.9057862373835 |
| BLK | 0.47681495061319 | 4.05228336851975 | 11.5055832638425 | 2.2158512177743 | 0.14076820970915 |
| BLNK | 0.219543810156093 | 0.0399140810419461 | 0.0521078343443318 | 0.156831295275187 | 8.43105468656622 |
| CD19 | 0.356327937281533 | 0.147968621036337 | 0.316340059188139 | 2.15657773549019 | 0.935245313853704 |
| CD200 | 0.0265141363076311 | 2.5800289522695 | 0.364691141932533 | 5.94285046438378 | 0.139223231944823 |
| CD72 | 40.681296144629 | 2.59695641001026 | 8.46289537578809 | 8.37780190378287 | 10.1659239858184 |
| EBF1 | 0.0586209590159303 | 0.0841256435057548 | 0.0951730200086187 | 0.225008749503655 | 0.0168898918049345 |
| FCMR | 160.801362342138 | 238.51602985363 | 169.884610521352 | 128.791016287648 | 4.06619509200073 |
| FCRLA | 0.145821049600758 | 0.0330421596518271 | 0.154749177923768 | 0.237167032398714 | 0.0509821884970258 |
| GNG7 | 4.05066447900761 | 5.73081622358209 | 5.58142045635171 | 5.09828868961245 | 6.44941623857556 |
| HLA-DOB | 18.9059199453473 | 16.3292481258742 | 14.8100847714772 | 20.3761854717362 | 17.3427969033096 |
| KLHL14 | 0 | 0 | 0 | 0.0373555099619273 | 0 |
| MEF2C | 3.14509288710368 | 0.0362806292160466 | 0.105976242400573 | 0.0982320517287213 | 42.7915023477713 |
| P2RX5 | 16.9231228737529 | 18.2779100466741 | 23.8854380497373 | 11.0092609428011 | 6.76903865761506 |
| PCDH9 | 0.748563297188226 | 0.0459008116068998 | 0.0814295234155041 | 0.0639128133460101 | 0.0426040936238881 |
| PNOC | 0 | 0.0472334678353536 | 0 | 0.309263402405445 | 2.13706065184964 |
| POU2AF1 | 2.4242039029227 | 2.68037765769087 | 1.70554137844238 | 5.66826032962433 | 1.99938078060004 |
| RALGPS2 | 8.21666028939979 | 5.83071669868946 | 5.17362767746268 | 3.68656104472208 | 5.52994894514008 |
| SPIB | 0.488686083753194 | 0 | 0.141958013991751 | 0.378468053776022 | 3.24352448341824 |
| STAP1 | 15.137255938093 | 5.71661900396787 | 1.7138338128214 | 0.759359030414403 | 0.351110200278079 |
| TCF4 | 5.59648314409974 | 0.149982882476814 | 0.280889508455173 | 1.43388340709661 | 6.96146309463079 |
| TCL1A | 0.0513603538737202 | 0 | 0.175558462420334 | 0.129041215983472 | 0.100432568101733 |
| VPREB3 | 0 | 0.0983907274710984 | 0.33391866565921 | 0.51018118066222 | 0.0789663494322397 |
| ACVRL1 | 0.428451188244593 | 0.129862974505345 | 0.267069845236772 | 0.666626998397525 | 1.09234822504098 |
| ADAMDEC1 | 0 | 0 | 0 | 0 | 0.236849813770509 |
| ALDH1A2 | 0 | 0 | 0 | 0.117068089614854 | 0.0458657199625384 |
| CCRL2 | 2.04129007638552 | 0.957673807099835 | 2.51730027777864 | 3.23743057641214 | 5.44466918719567 |
| CD86 | 0.0603402220417124 | 0.0760981608177539 | 0.188536589959567 | 0.0865791200124716 | 140.844033128846 |
| CFB | 0.189463391716347 | 0.298829768647172 | 0.202456383752594 | 7.22225726135961 | 3.19652157534609 |
| CXCL2 | 0.347510797495381 | 0.0690572869185464 | 0.184183175055644 | 0.767574713260437 | 7.1025906041116 |
| CXCL3 | 0.0764373381946375 | 0 | 0 | 0 | 2.22007793902901 |
| EREG | 4.13074188511085 | 0.872463173445688 | 1.21352611175253 | 4.60632409168193 | 3.53114879594062 |
| ICAM1 | 26.3225806061086 | 3.69508010525222 | 11.5612613899165 | 15.986869569612 | 36.1322728223818 |
| INHBA | 0.20000418280766 | 0 | 0.035747276231363 | 0.477624035862211 | 0.178770378218121 |
| LILRB4 | 0.725275080431343 | 0.0490182458691303 | 0.181584515477741 | 0.520041555026167 | 106.635462765275 |
| NUPR1 | 0 | 0 | 0 | 0.709012158561964 | 0 |
| PDPN | 0.63459380914047 | 0.162712173711314 | 0.202613723750939 | 0.939531337631743 | 0.347168317234732 |
| SERPING1 | 0.305133884924488 | 0 | 0 | 0.754461919408032 | 5.42399696368157 |
| SLC1A3 | 0 | 0 | 0 | 0 | 0.390851817530305 |
| SLC6A12 | 0.125382937018582 | 0 | 0.0596110059041317 | 0.489478964920557 | 9.90369164253591 |
| SQOR | 33.4404396709447 | 35.7465898070398 | 29.1984454186279 | 31.9313822498785 | 72.7178231004998 |
| VSIG4 | 0.25378754292522 | 0 | 0.286373784590677 | 0 | 5.16083076984105 |
| ACE | 3.16914580353855 | 8.97933308798429 | 6.66565749227824 | 5.13496022293496 | 8.62409948698236 |
| CISH | 19.1603796488508 | 15.6036929723125 | 11.7567208822068 | 4.26213645659397 | 7.74761118051773 |
| CLEC4A | 4.81850929843966 | 1.394315641778 | 1.4271109997333 | 3.23483676986554 | 89.0256998837511 |
| CLIC2 | 1.22132171269956 | 0.541267300470522 | 0.875651677000239 | 0.705357946746145 | 24.2207647644875 |
| COLEC12 | 0 | 0 | 0 | 0.0574930656175801 | 0 |
| ECM1 | 0.705727159671569 | 0.348000312233568 | 0.648906151183534 | 0.707579953444764 | 1.0856385983402 |
| ENPP2 | 0.660793376333027 | 1.08359402245564 | 0.601269588041625 | 0.123443624549889 | 1.43377461303016 |
| GATM | 0.800575597823844 | 0.0631246455604276 | 0.229462317519857 | 0.128905895618398 | 2.9684412010666 |
| GFRA2 | 0.465446244341031 | 0.258413822048901 | 0.0508338090906309 | 0.334103930283533 | 4.77493936405185 |
| IL1R1 | 1.88667111727635 | 1.52438865417414 | 0.386687176973508 | 5.53024419991008 | 2.70708875128472 |
| MAOA | 0 | 0.0125032470379778 | 0.0454643403057873 | 0.0391885031842564 | 0.0178614401119112 |
| MS4A4A | 0.654922083659694 | 0 | 0 | 0 | 62.824517858648 |
| PLTP | 5.76781005065948 | 0.193552235262807 | 0.725212589421827 | 1.31464816885043 | 4.57423452841977 |
| PTGS1 | 2.02625582522416 | 0.031677643711617 | 0.159772820629484 | 0.221839405913356 | 38.5115861058714 |
| RNASE6 | 2.57034224061085 | 1.55413621826526 | 0.755928891264297 | 0 | 62.6203374489481 |
| SLC7A8 | 0.310334431394085 | 0.832472050906884 | 1.83434770463846 | 0.608183780856691 | 0.363059196213789 |
| TREM2 | 0 | 0 | 0 | 0.293487576345749 | 0 |
| WNT5B | 0.0257615286791872 | 0 | 0 | 0.865764388560015 | 0.314581209761574 |
| CD1D | 0.252652767110177 | 0.247373650540447 | 0.202885755820835 | 0.359075844200055 | 67.5964214961601 |
| FXYD6 | 0 | 0 | 0 | 0.139995512052629 | 1.33426263346427 |
| GAS7 | 61.2730926695054 | 1.09417281111962 | 8.15823083234582 | 1.53088598986978 | 36.2498743535205 |
| HPSE | 1.96424981646418 | 6.47007322735653 | 3.56747341418466 | 4.89595787663339 | 4.98911096611645 |
| KBTBD11 | 0.678693638810208 | 4.48511612182804 | 2.03245735399911 | 3.04382537681445 | 6.87417954693535 |
| NRG1 | 0 | 0 | 0 | 0.242223160741488 | 0.814593946439049 |
| SMARCD3 | 6.81727686861426 | 13.8066611865776 | 22.8722104255595 | 4.58467417413573 | 20.9336784365503 |
| TMEM170B | 2.95644229394717 | 1.5514674206198 | 1.9499492717142 | 1.40404641978291 | 30.7360994681166 |
| ABHD5 | 6.90492928218358 | 2.47045921352886 | 2.95982783294023 | 6.42374466812199 | 9.24758721802321 |
| ADGRG3 | 18.2908053036202 | 0.0733607986856278 | 0.615943843013934 | 0.376632585531813 | 0.305176850952508 |
| BTNL8 | 0.479883817241601 | 0.158883147138341 | 0.228664581398865 | 0.389246030903637 | 0.691525632497091 |
| CEACAM1 | 4.66892245154762 | 5.68918318884606 | 2.80889457471759 | 7.14074535916833 | 12.0611433565834 |
| DAPK2 | 6.80556737844844 | 1.3791998358232 | 3.26502596754617 | 2.77397194707662 | 1.97127899697657 |
| DGAT2 | 0.284534171295842 | 0.105764334237416 | 0.199429446468928 | 0.186774693982638 | 4.24676158538125 |
| DPEP3 | 1.98268277650309 | 1.0559804494746 | 1.33494451788636 | 1.09473669328588 | 2.3776573969495 |
| DYSF | 11.3840128235166 | 0.103514194434563 | 4.16142977523703 | 0.212271150442656 | 3.4868079835584 |
| FPR2 | 0.68117408211071 | 0.0513704529427299 | 0.0289443713323537 | 0.116065287115604 | 32.3578631768778 |
| KCNJ15 | 0.148403005246877 | 0.0657015475775988 | 0.0247869751659843 | 0 | 0.0382922964239043 |
| LILRA2 | 5.57695128498415 | 0.0881305924702437 | 0.243840281109146 | 0.524084574382944 | 287.267517957501 |
| LRRC4 | 1.06878195792169 | 0.0992332115370332 | 0.495925422318774 | 0.102350769617004 | 9.64418514529133 |
| MGAM | 14.6226160451376 | 0.0361756814742134 | 0.135863299590202 | 0.0314131507771003 | 0.0673647449302766 |
| MME | 0.0909303358144165 | 0.113295410234375 | 0.102228493326538 | 0.108821895225172 | 0.0153137845096485 |
| REM2 | 8.71505768885506 | 7.91170273671624 | 5.30907305650755 | 45.0947045269146 | 1.27822351668252 |
| REPS2 | 0.992161337730061 | 0.505194326190745 | 0.335317354777063 | 0.656181780537616 | 4.89323694665316 |
| TMCC3 | 14.534413642262 | 0.346112750767616 | 1.85519257378848 | 0.243206172614697 | 9.19131828004531 |
| ABCB1 | 35.6508877086871 | 2.11898303280204 | 9.28339632057 | 0.454617179592002 | 0.082439291645955 |
| AUTS2 | 66.7609172040971 | 11.1075589050532 | 14.2835799205952 | 1.58177171037035 | 1.00056493747498 |
| COLGALT2 | 16.9619578121379 | 0.240984142843278 | 1.28494809224896 | 0.139243037830348 | 0.234177907775057 |
| EOMES | 34.7156343600852 | 1.30604735192787 | 14.3770029468759 | 0.151428593146513 | 0.25216006042113 |
| FASLG | 40.8492412586392 | 0.623718027569546 | 2.98019012348235 | 0.71212345904445 | 0.441441156866181 |
| GK5 | 133.46318029013 | 12.3841164304741 | 21.0728335858943 | 11.9032358614986 | 21.6937690965472 |
| GNG2 | 347.443875680483 | 83.4073676703523 | 94.1001706756385 | 87.8178091927617 | 141.304391970948 |
| HOPX | 468.478658307824 | 15.5377357384362 | 57.4276028639375 | 1.61089698719135 | 5.8914750536465 |
| KLRD1 | 246.290655037427 | 2.02126613443466 | 20.1772202718613 | 4.63185207418001 | 4.43383028233963 |
| MATK | 530.630876675417 | 11.0694601307497 | 106.17134025207 | 1.37210109748567 | 1.96233698366967 |
| MLC1 | 113.094524217669 | 0.1296925507579 | 1.72942783244647 | 0.311566324010531 | 22.4326964772497 |
| NCALD | 47.2777278774844 | 4.73280850210663 | 11.2361172230354 | 3.31577762610745 | 0.718316718859927 |
| NLRC3 | 173.707612366465 | 67.8169579284791 | 81.097680777408 | 81.1537468189753 | 1.38381225093569 |
| PDGFD | 42.3123568410012 | 0.0617097282978706 | 4.08244985369889 | 0 | 0.149292404546203 |
| PLEKHF1 | 136.220494481129 | 19.7601735328878 | 36.3875036714117 | 19.4829374061383 | 0.35839486365219 |
| PPP2R2B | 39.0032550123116 | 3.51475258905942 | 13.5026972043889 | 3.21225579522072 | 0.253709164352637 |
| PYHIN1 | 261.730478134421 | 40.2737734165784 | 55.2585693696872 | 67.9548105795143 | 0.774951434334451 |
| RNF165 | 35.5434340680214 | 0.117728247923624 | 0.887761088278177 | 0.252740042233168 | 0.081893802886078 |
| S1PR5 | 461.622655883738 | 1.73149267288106 | 41.7720941125901 | 0.529389171769584 | 0.941674082801863 |
| SAMD3 | 367.321483262895 | 57.8665949411827 | 120.297178579186 | 19.9764968089219 | 2.28183641457969 |
| SBK1 | 66.5865700183104 | 16.6923511976197 | 27.4766211450453 | 11.0229110496692 | 0.878348789486539 |
| SH2D2A | 318.736817568831 | 36.7051553439723 | 66.4500413369875 | 28.9077019899112 | 0.586233070168583 |
| SLFN13 | 81.3551353936778 | 9.88957363429813 | 14.6940935708963 | 7.71790489678105 | 1.80270414062867 |
| STAT4 | 256.875614024983 | 73.2041216492301 | 123.022378087081 | 80.1857788989678 | 3.16596535100721 |
| TOGARAM2 | 62.6412276380914 | 1.93892670476424 | 5.95616893994568 | 6.08552759746374 | 0.979826510624534 |
| TSPOAP1 | 298.272861769601 | 11.825345994764 | 32.5275419110664 | 56.2183401041487 | 5.95571687968183 |
| TTC38 | 325.703098369836 | 4.25650122194311 | 21.6000422452249 | 3.29072865422217 | 13.9440199161427 |
| TXK | 289.240921035468 | 71.0453406251931 | 82.3552864784903 | 41.5394225337838 | 3.26555028659687 |
| YPEL1 | 105.166800704502 | 5.96877974833461 | 14.8377005837951 | 3.98226370462469 | 4.40046034156515 |
| ZBTB16 | 86.352627233819 | 4.85074087899058 | 12.1416121787729 | 2.9900499195532 | 7.3411866820438 |
| FHIT | 6.12221931010403 | 98.2539901326223 | 17.2088952793353 | 11.9337879122037 | 1.98969687632555 |
| TRAT1 | 1.82527686234142 | 116.662055071454 | 42.5594408230054 | 45.8588697175739 | 0.0669900605780553 |
| TSHZ2 | 0.077094463755214 | 10.3402256706886 | 0.530722197270164 | 6.07833167161168 | 0.0355205210753674 |
| TSPAN18 | 2.76885689467624 | 50.120140979035 | 14.0372256147086 | 4.73324441237652 | 0.434106047809428 |
| WNT7A | 0 | 17.0883963820579 | 4.57635871659645 | 0.606321578184723 | 0 |
| CD8A | 5.16732843080116 | 1.99249727130834 | 538.216797539064 | 1.22352364938439 | 0.218412818059309 |
| CD8B | 5.63372501931945 | 4.94154824531243 | 690.196951723652 | 6.55912029872283 | 2.72207773905785 |
| FCGBP | 4.87186057602238 | 2.60228020137674 | 13.8271423408746 | 1.01047909707915 | 1.15535396150461 |
| CCR4 | 0.0347657920943443 | 58.0676088483504 | 7.43907327707358 | 150.751192708116 | 0 |
| CD5 | 0.8921825570264 | 193.010470187532 | 130.797307511882 | 317.912181219475 | 4.30120434495132 |
| CTLA4 | 0.46586245398606 | 49.5392798571527 | 8.40674447708265 | 325.121345120445 | 0.362548596458703 |
| FAM160B1 | 27.6647241983289 | 15.3767834408427 | 18.7547777958565 | 86.397493390073 | 8.53151465501255 |
| FAM46C | 105.655349650099 | 95.0297332666513 | 98.5188929497421 | 276.884033768097 | 0.153696326240277 |
| FOXP3 | 0.93322145283811 | 24.9789912227117 | 1.75647197347746 | 313.685821002479 | 0.816092788714517 |
| GCNT4 | 0.521541094717927 | 13.5084629276821 | 6.37936890233892 | 27.0767641549312 | 0.299027573535988 |
| HPGD | 8.86691928763713 | 5.43663408188655 | 8.74481612071862 | 94.5114858522998 | 0.331635152719544 |
| ICOS | 0.961526295821154 | 115.115985214873 | 36.1140724223231 | 260.934011437143 | 0.15560452283748 |
| LAYN | 0.920713691127399 | 0.495056559308106 | 1.516722992923 | 23.3669409859553 | 2.52077384568442 |
| LRRC32 | 0 | 1.26875800849278 | 0.0558000204903282 | 26.8628640411678 | 0.106702947830908 |
| PLCL1 | 0.361016216391006 | 16.9077427846048 | 5.58950922758938 | 46.2798126734051 | 2.13598032853746 |
| RTKN2 | 3.23745014567294 | 13.3831296624276 | 4.52248414125275 | 105.39655850358 | 2.07597260602569 |
| SEMA3G | 0.0115186632413928 | 1.36963688321624 | 0.0837724540300752 | 13.6866231859119 | 0 |
| AATBC | 0.862381185603521 | 0.169612370993582 | 0.395075723327132 | 1.51582707226835 | 122.670067089138 |
| FZD1 | 0.0909306042602444 | 0.206749189436629 | 0 | 0.0431761999161938 | 47.5719235812834 |
| GRK3 | 1.68403817993922 | 1.35750453706955 | 1.28712027060365 | 4.64167585685216 | 116.14265581675 |
| GUCY1A3 | 4.36278835223315 | 0 | 0.0290212664133315 | 0.125262148775337 | 17.560921932425 |
| KDM1B | 9.94998918107382 | 6.19604745256449 | 5.86026195214282 | 5.63208170344591 | 144.194033835313 |
| LMO2 | 1.63912146426352 | 0 | 0 | 0.242979350174535 | 298.59719763019 |
| RNF144B | 1.46579176099642 | 1.63738885565927 | 1.30452647836947 | 0.891397177214894 | 151.249057000754 |
| SLC2A6 | 14.0815583690715 | 1.95998168347342 | 8.79203361079547 | 3.46983634170838 | 315.921537472941 |
| TTYH2 | 5.02131345974304 | 11.367234453737 | 6.97316248163613 | 6.566632857657 | 107.498219237942 |

TABLE 4
Exemplary 170-gene signature matrix

| | | M1 | M2 | | |
|---|---|---|---|---|---|
| Gene | B cells | Macrophages | Macrophages | Monocytes | Neutrophils |
| ABCB4 | 22.7138212901797 | 2.23179950051687 | 1.97155445562159 | 0.105994738308192 | 0 |
| ADAM28 | 138.122317697073 | 44.549173431875 | 4.86154374716257 | 6.15419538796811 | 6.92361545168478 |
| ADAM6 | 311.524313608549 | 0.185500319951345 | 0.387193475322115 | 0.0309434181627899 | 0.0313699237303561 |
| AFF3 | 74.3798680534155 | 1.41947594130957 | 1.79918141862698 | 2.90259697885655 | 1.79219821564678 |
| AKAP2 | 90.5380895821784 | 17.5227274563533 | 3.32632182691764 | 0.221090667019584 | 0 |
| ARHGAP24 | 48.923113469842 | 10.4272519545846 | 2.52236669354933 | 17.6398417143727 | 3.12847520545414 |
| BANK1 | 301.24413442937 | 4.00037342190056 | 7.12162949184432 | 2.43007530175162 | 0.0467090734061819 |
| BCL11A | 162.35746258436 | 2.02454979826139 | 9.5209120731403 | 21.7927458929775 | 1.74970688550885 |
| BLK | 529.702738900353 | 0.804533642108712 | 0.725478229136685 | 0.162953576261731 | 0.112842610698866 |
| BLNK | 174.401225832058 | 2.41629459859149 | 61.5054350055487 | 1.56419570294049 | 0.0488792691033357 |
| CD19 | 439.268676213828 | 0.940667995897954 | 0.960748363506361 | 0.339789686883238 | 0.105206787085179 |
| CD200 | 55.2294664656558 | 0 | 1.41548056084562 | 0 | 0.662093077794551 |
| CD72 | 258.499972469939 | 12.484112301443 | 4.9256997230085 | 3.4455066344001 | 0.150684735222654 |
| EBF1 | 33.9402843306255 | 0.0419617982650429 | 0.107810010150852 | 0.0258455531259009 | 0.0263452108172489 |
| FCMR | 560.208427058502 | 4.33144930960849 | 1.48636123601938 | 0.38458372366092 | 11.0654267178681 |
| FCRLA | 240.512072319375 | 1.50019097388064 | 0.619542712060854 | 0.040850234118554 | 0 |
| GNG7 | 120.485122831339 | 3.36311354485396 | 5.80324654779615 | 4.72007086693286 | 13.7628682574527 |
| HLA-DOB | 372.459806363368 | 7.07984181889178 | 3.25821368357222 | 9.5537257936507 | 15.7027822745319 |
| KLHL14 | 37.7430063578197 | 0 | 0 | 0 | 0 |
| MEF2C | 116.323311328885 | 5.89045977701664 | 11.1354921599087 | 31.5715802571576 | 7.85772776063656 |
| P2RX5 | 635.399774973654 | 1.81920580725339 | 1.53569999555268 | 5.36565883803721 | 4.20649120944489 |
| PCDH9 | 25.1154006032258 | 0.0471608297055587 | 0.0346111724453279 | 0.0135109763504724 | 0 |
| PNOC | 102.303071324944 | 0.481037399096269 | 0.221787282606074 | 3.674790248062 | 0 |
| POU2AF1 | 210.360922846279 | 1.07402574055616 | 1.02531762861916 | 1.25038125930661 | 1.42186066661587 |
| RALGPS2 | 243.443027497961 | 5.06750774949951 | 14.4045255072051 | 4.23115886707842 | 11.6209486813389 |
| SPIB | 224.463150722433 | 0.482360222996223 | 0.70036408031993 | 0.141625346838359 | 0.0518068448663745 |
| STAP1 | 138.998205062899 | 0.75443517176183 | 1.78548158989623 | 0.137633946325403 | 0.174061069300259 |
| TCF4 | 66.326842586527 | 3.68019267419876 | 26.7980773610538 | 5.19195703557171 | 0.198946422341884 |
| TCL1A | 551.872721659835 | 0 | 0 | 0 | 0.0461283203003378 |
| VPREB3 | 288.520510513099 | 0.494539146107356 | 0 | 0 | 0 |
| ACVRL1 | 0.332585294715274 | 19.7772120444697 | 2.10520049097964 | 1.58765673970166 | 0.424882227546612 |
| ADAMDEC1 | 0.635314747578684 | 183.705448066853 | 9.49282348979648 | 1.20280845755159 | 0.054395364353221 |
| ALDH1A2 | 0 | 23.1067632472012 | 9.7981847931226 | 0.123003597462278 | 0.571071038124148 |
| CCRL2 | 1.49069074245513 | 68.3022602497262 | 8.21384716771491 | 14.8175992642263 | 3.39577878674019 |
| CD86 | 14.8419681265522 | 415.559752234066 | 141.598162873142 | 124.373970474268 | 0.0260916172816413 |
| CFB | 0.152608724773573 | 65.9967893726307 | 17.9938750647176 | 1.51149883576967 | 0.231942366645218 |
| CXCL2 | 0.109297174031566 | 302.903768620453 | 34.1076360418172 | 47.1810943911007 | 2.43196954555697 |
| CXCL3 | 0 | 259.154144878705 | 51.7012610500732 | 6.61824121804823 | 0.422991300148712 |
| EREG | 1.426795625176 | 163.050043614166 | 15.6097902146948 | 23.7225799821433 | 1.39213256974851 |
| ICAM1 | 6.17602138151068 | 592.921253202952 | 207.459056399526 | 125.338706945171 | 121.463688878235 |
| INHBA | 0.0639257465975131 | 578.149564143944 | 21.2499318026688 | 0.203242228223779 | 0.345224461315037 |
| LILRB4 | 3.78654567485434 | 476.82255473671 | 133.464950198138 | 124.041784829028 | 18.5815069394489 |
| NUPR1 | 0 | 101.322179479238 | 22.4329877889438 | 0.0882116265122513 | 0.0811838909277743 |
| PDPN | 0.263864646113126 | 16.776474481883 | 6.31494558426671 | 0.370034834512035 | 1.56290059271976 |
| SERPING1 | 0 | 500.333873998758 | 17.3112787031335 | 27.543535044725 | 77.6732315399919 |
| SLC1A3 | 0.0356287531145159 | 25.7591310157418 | 5.95100851102093 | 0.927036634840135 | 0.0340795710863373 |
| SLC6A12 | 0 | 40.8791807642813 | 1.66954056123124 | 6.85529805658659 | 3.24619240517486 |
| SQOR | 26.5317324389891 | 535.536312247838 | 177.585739894353 | 126.37905571682 | 191.98901026902 |
| VSIG4 | 0.109938272500894 | 287.034988195206 | 8.91663291464562 | 8.59671456395031 | 1.20698377612712 |
| ACE | 3.7992101423842 | 92.2488019058023 | 303.464970506693 | 2.53539965563725 | 3.34883479329738 |
| CISH | 0.737194776103433 | 103.895600398754 | 239.503450756414 | 4.85970644061934 | 8.5357260446685 |
| CLEC4A | 10.4771933509048 | 71.6302696769377 | 400.766557876869 | 149.530636617981 | 135.985920646415 |
| CLIC2 | 0.694483626998776 | 30.7996880352197 | 133.692558026865 | 5.06218812509425 | 2.25505284322546 |
| COLEC12 | 0 | 23.2936430965653 | 78.8953739723945 | 1.16683303498152 | 0 |
| ECM1 | 1.16224794525222 | 25.2617977050096 | 51.7673538087741 | 0.851277964741539 | 0.125565820900312 |
| ENPP2 | 0.798089326413484 | 23.2877128002904 | 59.124417778249 | 1.00560930051376 | 0.241410922855034 |
| GATM | 6.24630433858738 | 1.30201783504023 | 113.238809577875 | 1.02984423835886 | 0 |
| GFRA2 | 0.130888080063566 | 4.83084435375596 | 79.2607177166156 | 2.06724773612502 | 0 |
| IL1R1 | 0.51108490301588 | 30.803556063969 | 212.912935199361 | 2.3804779784133 | 25.4042245502966 |
| MAOA | 0 | 5.95645516617961 | 80.1465476825236 | 0.103115807610358 | 0 |
| MS4A4A | 0.13127327186729 | 30.3792176392599 | 212.758993973887 | 12.9864959847751 | 0 |
| PLTP | 4.09773209191575 | 15.5762699043529 | 90.072255489694 | 2.13834344792328 | 0.631419406467024 |
| PTGS1 | 17.7102769818648 | 23.5081781658257 | 111.231364417806 | 26.2406466909419 | 5.36821745232743 |
| RNASE6 | 64.1140064772035 | 206.972276626839 | 633.832133210133 | 201.794703201243 | 45.1798115742456 |
| SLC7A8 | 0.237455366576669 | 12.0565466071266 | 87.1386494103188 | 0.292623094712473 | 0.0562275499429652 |
| TREM2 | 0.0436228969244222 | 25.0828674728233 | 204.084827382242 | 0.719506568957995 | 0.330206010339332 |
| WNT5B | 0.124075186063689 | 0.373209145686068 | 21.0485792500978 | 1.53606464649534 | 0.0285119129727064 |
| CD1D | 22.9579813644177 | 3.34560477744368 | 1.12160518738656 | 109.71255533243 | 4.25317725185491 |
| CD36 | 0.729067770678925 | 21.9856422902364 | 124.549538548757 | 457.723033207541 | 1.06041549651452 |
| CSTA | 0 | 151.612186784145 | 99.6793558120111 | 417.399735727219 | 173.314848407162 |
| FXYD6 | 0.0499479108396515 | 8.14251465517473 | 1.51808441059495 | 51.1474894647036 | 0.163029810701658 |
| GAS7 | 3.05073991381932 | 38.413665207398 | 35.0489843958423 | 109.921417466546 | 32.3679507043919 |
| HPSE | 1.17369291780026 | 2.9430035890169 | 2.29581082691957 | 77.9788201672732 | 35.4940239786558 |
| KBTBD11 | 0.384689196355843 | 4.84433108833272 | 6.5406673151378 | 11.5850984842579 | 4.85401131707557 |
| NRG1 | 0 | 0 | 1.01807919863294 | 35.4297965389362 | 0 |
| NRGN | 2.9489736858828 | 7.55676796532373 | 31.931952483916 | 232.977958460721 | 3.78599733916265 |
| SMARCD3 | 4.59071900115077 | 15.6068632596657 | 10.643282677309 | 152.811686007079 | 23.7139348285711 |
| TMEM170B | 2.45693897803563 | 24.6567425931697 | 8.88862444506901 | 77.1849568226254 | 10.9176069559463 |
| ABHD5 | 2.4032915401641 | 30.1571858535619 | 13.1805019532929 | 18.5733113292649 | 68.0693895185095 |
| ADGRG3 | 0.131466148745403 | 0.472304260413813 | 0.152058295813748 | 0.538263293930684 | 571.897708115885 |
| BTNL8 | 0.368149861441642 | 0.302261559138706 | 0 | 0.800757200154997 | 104.95658070008 |
| C5AR2 | 1.18752287266006 | 25.225382519178 | 11.6911687730329 | 26.2987199724024 | 214.87346345783 |
| CEACAM1 | 6.87952133740813 | 1.94479031483905 | 1.36810402032269 | 2.96191872941251 | 74.5043059312033 |
| CEP19 | 2.69660634902883 | 2.63660991582848 | 3.26492995565722 | 3.01970867364809 | 61.5766711771006 |
| CYP4F3 | 0.0455002475253714 | 0 | 0.142043524108379 | 1.68987491572689 | 245.508507701855 |
| DAPK2 | 5.07134007453929 | 1.13104439724161 | 0.816545662777064 | 2.23079479124051 | 100.413351085799 |
| DGAT2 | 3.82158723795715 | 7.67644326641274 | 6.2186171673132 | 6.58444059177283 | 327.49380508872 |
| DOCK5 | 0.787872938943236 | 24.7898999968362 | 14.6268954057184 | 24.0501437880117 | 147.740498112033 |
| DPEP3 | 2.59260003814738 | 0.533960079085381 | 1.93834876262687 | 1.66611711910439 | 34.8086167483078 |
| DYSF | 0.141021184240563 | 14.9194244156264 | 7.23975607048224 | 43.5038034974759 | 333.701448218021 |
| FPR2 | 0.0888493853574401 | 41.6179915042076 | 4.47574697178295 | 39.2481280641213 | 669.095997076219 |
| HAL | 1.3699035881031 | 2.25939756618088 | 1.80486497093235 | 18.9841835923287 | 133.805375403618 |
| KCNJ15 | 0.034450621804724 | 20.9510319819367 | 1.07629254998603 | 3.98484698454836 | 361.827478544255 |
| LILRA2 | 0.947022274267314 | 90.3026256558375 | 8.07048363222991 | 288.439645020056 | 607.112566405046 |
| LRRC4 | 0.617728719549034 | 0.214277039161762 | 1.38891714902995 | 3.78613786610382 | 85.9786297945884 |
| LRRK2 | 17.0044330287365 | 96.1583214422161 | 8.78053490907599 | 68.3361333640722 | 352.222718212421 |
| LY96 | 12.038709280056 | 98.3163148242062 | 157.387616081097 | 85.7470004246651 | 363.813671910055 |
| MGAM | 0.0471277210657418 | 8.85197804080068 | 0.407997709201937 | 3.37314275253331 | 319.245026714378 |
| MME | 1.29490791514107 | 0.558026968296873 | 0.170019176920162 | 0.538787242608576 | 382.109462967198 |
| NINJ2 | 4.58631635161128 | 2.21231236500275 | 7.33460735457702 | 24.4808421326114 | 63.8610951640169 |
| PPP1R3B | 4.89295383097494 | 11.0248479501875 | 35.6283627940233 | 3.29295537954135 | 109.844000651765 |
| REM2 | 12.3258997018747 | 1.75338875771919 | 2.47587455119383 | 1.27872108996671 | 81.4152262930432 |
| REPS2 | 2.24282263338729 | 0.954439466148988 | 10.1087744474924 | 3.43139437886716 | 32.7349262478794 |
| TECPR2 | 4.8505776168776 | 4.96668623028505 | 7.50582995058025 | 9.81351174862256 | 99.231876185812 |
| TLR1 | 18.8135342514735 | 95.1766432641611 | 36.6284795593902 | 32.5743229543974 | 252.980840768643 |
| TLR4 | 1.34959261649303 | 76.3362246511968 | 80.1866266684685 | 111.355385075263 | 209.46495239145 |
| TMCC3 | 6.57322429545358 | 8.14793681989921 | 6.12198592655159 | 6.33101986919401 | 59.9283524527154 |
| TMEM154 | 41.6665031870547 | 3.60419709708908 | 2.49809559963814 | 51.5739498092694 | 419.423571763283 |
| ABCB1 | 4.63154686733255 | 0.715223790192447 | 0.287480957998641 | 0 | 0 |
| AUTS2 | 10.4292142934928 | 0.378491284950299 | 0.70563506554458 | 0.350534093801845 | 0.0468427807798921 |
| COLGALT2 | 0.0503339008113001 | 0.691005435055901 | 2.83892801522651 | 0.0427929381517172 | 0.0235958819004023 |
| EOMES | 0.0294414936808075 | 0 | 0 | 0 | 0 |
| FASLG | 0.406052635272513 | 0.152148329940168 | 0.0619085809277628 | 0.354963620298727 | 0.165275606590322 |
| GK5 | 10.4441946202148 | 13.9822172169163 | 8.68370624717098 | 10.6140157840911 | 2.65354163119785 |
| GNG2 | 39.8257313163402 | 4.58086240903197 | 16.6046687273083 | 52.8962086615344 | 65.4887065821961 |
| HOPX | 17.2321146202866 | 3.16965457911556 | 1.47623189663556 | 0.131760621540902 | 0.235345152176187 |
| KLRD1 | 2.1730920691707 | 1.33460636734408 | 1.72022815373913 | 1.66168543626761 | 1.648031188641 |
| MATK | 5.0697317486215 | 65.4926135332061 | 45.4487195090692 | 0.304760770589686 | 0.192240973293835 |
| MLC1 | 0.0485219973466009 | 0.0641268697938566 | 0.538831709037623 | 2.68953562519948 | 0.264360540457374 |
| NCALD | 0.0436602836343503 | 0.0742748478569402 | 0.0289257130114139 | 0.0327413298290547 | 0.0158724306087475 |
| NLRC3 | 41.0377217209724 | 1.30118117064042 | 0.760087584031309 | 0.724039617208233 | 2.34517996463339 |
| PDGFD | 0.235737723867995 | 0.0381807935343397 | 0.0807138003344366 | 0 | 0 |
| PLEKHF | 2.89906019044632 | 5.57735836943224 | 1.31936324244112 | 0.155325651351853 | 0.0677775107833816 |
| PPP2R2B | 0.0562970804183874 | 0 | 1.00444928865373 | 0.0983155589802625 | 0.121169066030033 |
| PYHIN1 | 10.8375867804319 | 1.13507144800805 | 0.481226732421874 | 0.172259027619941 | 0.672550480103105 |
| RNF165 | 0 | 0 | 0.0219641170576724 | 0.048744954610262 | 0.0243795407887355 |
| S1PR5 | 0.0618605571367731 | 0.074065425890921 | 0.312335075546808 | 0.100674680009297 | 0.0285574435648188 |
| SAMD3 | 3.15766899105223 | 0.768173205038007 | 0.423872667811168 | 0.730838797177124 | 0.451777467510685 |
| SBK1 | 0.89683364209223 | 0.244026650940044 | 0.107697845591846 | 0.273248007380871 | 0.313675097000316 |
| SH2D2A | 0.417387642431526 | 0.399430552086268 | 1.0692672878798 | 0 | 0.0869516539121953 |
| SLFN13 | 4.93323201574845 | 2.98630116225055 | 3.19026545673749 | 0.968729048124671 | 1.22170364547377 |
| STAT4 | 3.5137613091855 | 11.4359609879083 | 6.86205037725543 | 1.31034705378228 | 1.68724203860777 |
| TOGARAM2 | 0.14977604730489 | 0.682597729197908 | 1.38778238309787 | 1.48423663565902 | 0.275934616545652 |
| TSPOAP1 | 5.13086518690353 | 5.07246402179412 | 2.13312627831793 | 11.4858669883818 | 3.0399898258509 |
| TTC38 | 3.85182444236592 | 9.26844279463642 | 13.4183090394676 | 14.3877671325781 | 4.57582303947228 |
| TXK | 1.67435589560267 | 1.7969541080826 | 1.81590794963782 | 1.27335372931424 | 1.18412287042393 |
| YPEL1 | 23.9202438542052 | 1.75716237532657 | 7.6419948072116 | 1.71145377856093 | 1.90757394112732 |
| ZBTB16 | 14.2955085383565 | 0.562626343097506 | 0.535758215179425 | 8.474397823996 | 1.24492386029471 |
| FHIT | 3.33523058079424 | 4.08008791045851 | 5.79890885711685 | 3.61138210623977 | 0.31545962658046 |
| TRAT1 | 0.0642279284285004 | 0.175857133819589 | 0.643153242744908 | 0 | 0 |
| TSHZ2 | 0.282395365806985 | 0.0694927864465123 | 0.0737068227574146 | 0.0143946971064045 | 0.28327099666231 |
| TSPAN18 | 1.77311089111071 | 0.224973142556574 | 0.299670125973253 | 0.303297482065292 | 0.282454969067344 |
| WNT7A | 0 | 0 | 0 | 0 | 0 |
| CD248 | 0.259603865826594 | 0.0877400678971564 | 0.299374045396017 | 0.0428499939905287 | 0 |
| CD8A | 0.123493752001712 | 0.611395299403943 | 0.53185577970834 | 0.0776055292528393 | 0.104437399218878 |
| CD8B | 2.29233359395268 | 1.80448168335034 | 3.84684462269583 | 1.28711293298135 | 2.29857071513904 |
| FCGBP | 4.19498815568179 | 0.0449441940887562 | 0.0571257486083144 | 0.0989068734123658 | 0.00501899302867541 |
| CCR4 | 0.127506702116757 | 0 | 0.30773593977729 | 0 | 0 |
| CD5 | 4.87737029057527 | 2.23528835363798 | 1.64234966363638 | 0.453178382198983 | 1.14838938173163 |
| CTLA4 | 0 | 0.939719253999237 | 1.14540488404531 | 0.0845014947257681 | 0 |
| FAM160B1 | 11.6727963252047 | 16.3066269771222 | 18.7870913232553 | 11.5574787297536 | 35.6428461439641 |
| FAM46C | 116.520091293196 | 0.786458366097308 | 1.02949567580101 | 0.121436258116005 | 3.58577926298475 |
| FOXP3 | 0.966707150988455 | 0.572910937281857 | 0.82573435848077 | 0.599754930500575 | 0 |
| GCNT4 | 0 | 0.128566977556141 | 0.0941949576915401 | 0.0129487954235771 | 0 |
| HPGD | 0.297346138478579 | 2.40675869902318 | 0.423144116281786 | 0.437543775657933 | 1.26252571799256 |
| ICOS | 0 | 0.293128153030223 | 0.256209509065337 | 0.0176882892358036 | 0 |
| LAYN | 0 | 0.0835626075128009 | 0.587878540672166 | 0 | 0 |
| LRRC32 | 1.92104533534765 | 10.2017663613868 | 0.837196973744059 | 0.599752724112324 | 0.0416687651863303 |
| PLCL1 | 0.104985030369203 | 2.44685686557505 | 5.08020913098262 | 1.62446678236619 | 2.16952878835384 |
| RTKN2 | 3.57035800815153 | 1.00177009348764 | 1.12522533615193 | 1.77421111129307 | 2.11788949245385 |
| SEMA3G | 0.0389765740960512 | 0.577584859041355 | 1.07254018565268 | 0.0354487377226781 | 0 |
| AATBC | 0.446909421795834 | 1.54300657250473 | 4.90994279344639 | 27.1038477887334 | 0.239048944286304 |
| FZD1 | 0.388966762996538 | 13.7784554207995 | 14.8284535753208 | 14.0384856679828 | 0.0469713753506456 |
| GRK3 | 45.9327801347347 | 49.0179285100589 | 66.8058033822207 | 61.5685769015622 | 3.4363986793443 |
| GUCY1A3 | 0.568909387506678 | 0.191756590854844 | 0 | 0.404019093331346 | 0.0148675306736854 |
| KDM1B | 14.9453686150132 | 59.9568838934326 | 46.529497052511 | 64.3965304809244 | 38.7289987387649 |
| LMO2 | 12.1479226527841 | 65.7025664762441 | 116.938733709168 | 108.097739911285 | 73.589743363124 |
| RNF144B | 7.19277800956162 | 40.058627103598 | 32.4435389643522 | 36.9045827477536 | 52.946478435322 |
| SLC2A6 | 62.219054604372 | 37.1231330527007 | 24.7683926116702 | 99.00556540819 | 1.27878370982374 |
| TTYH2 | 2.12221597740415 | 6.61923959128669 | 22.4669511772236 | 43.3162169610057 | 0.910160868995527 |

| | NK | CD4 T | CD8 T | | Dendritic |
|---|---|---|---|---|---|
| Gene | cells | cells | cells | Tregs | cells |
| ABCB4 | 0.236595831817282 | 0.0419535587353655 | 0.107002104922173 | 0.046410247442641 | 2.14790312131434 |
| ADAM28 | 16.0381494634342 | 0.0848609886518498 | 0.0793306796491496 | 0.662666725813814 | 18.9158947025691 |
| ADAM6 | 0.501235722794415 | 0.480615237724707 | 0.390699771827651 | 0.31171570243624 | 0.0777842223142737 |
| AFF3 | 5.31305820583621 | 3.33874242925966 | 1.6719953555597 | 3.41073423545352 | 9.31677002188692 |
| AKAP2 | 6.84541044568527 | 5.8905594487714 | 1.27121634621691 | 18.3633991365087 | 3.30470201093971 |
| ARHGAP24 | 0.052504487033627 | 0.0106536459980673 | 0 | 0.21506860740811 | 2.34976503501111 |
| BANK1 | 1.14720902003051 | 0.0516732285128874 | 0.158108993406954 | 0.0695917288799809 | 4.13538374563377 |
| BCL11A | 0.328324746898523 | 0.333422102683569 | 0.306429474503156 | 0.129487166590795 | 24.9057862373835 |
| BLK | 0.47681495061319 | 4.05228336851975 | 11.5055832638425 | 2.2158512177743 | 0.14076820970915 |
| BLNK | 0.219543810156093 | 0.0399140810419461 | 0.0521078343443318 | 0.156831295275187 | 8.43105468656622 |
| CD19 | 0.356327937281533 | 0.147968621036337 | 0.316340059188139 | 2.15657773549019 | 0.935245313853704 |
| CD200 | 0.0265141363076311 | 2.5800289522695 | 0.364691141932533 | 5.94285046438378 | 0.139223231944823 |
| CD72 | 40.681296144629 | 2.59695641001026 | 8.46289537578809 | 8.37780190378287 | 10.1659239858184 |
| EBF1 | 0.0586209590159303 | 0.0841256435057548 | 0.0951730200086187 | 0.225008749503655 | 0.0168898918049345 |
| FCMR | 160.801362342138 | 238.51602985363 | 169.884610521352 | 128.791016287648 | 4.06619509200073 |
| FCRLA | 0.145821049600758 | 0.0330421596518271 | 0.154749177923768 | 0.237167032398714 | 0.0509821884970258 |
| GNG7 | 4.05066447900761 | 5.73081622358209 | 5.58142045635171 | 5.09828868961245 | 6.44941623857556 |
| HLA-DOB | 18.9059199453473 | 16.3292481258742 | 14.8100847714772 | 20.3761854717362 | 17.3427969033096 |
| KLHL14 | 0 | 0 | 0 | 0.0373555099619273 | 0 |
| MEF2C | 3.14509288710368 | 0.0362806292160466 | 0.105976242400573 | 0.0982320517287213 | 42.7915023477713 |
| P2RX5 | 16.9231228737529 | 18.2779100466741 | 23.8854380497373 | 11.0092609428011 | 6.76903865761506 |
| PCDH9 | 0.748563297188226 | 0.0459008116068998 | 0.0814295234155041 | 0.0639128133460101 | 0.0426040936238881 |
| PNOC | 0 | 0.0472334678353536 | 0 | 0.309263402405445 | 2.13706065184964 |
| POU2AF1 | 2.4242039029227 | 2.68037765769087 | 1.70554137844238 | 5.66826032962433 | 1.99938078060004 |
| RALGPS2 | 8.21669979602893 | 5.83078946166986 | 5.17362767746268 | 3.68656104472208 | 5.52994008489451 |
| SPIB | 0.488686083753194 | 0 | 0.141958013991751 | 0.378468053776022 | 3.24352448341824 |
| STAP1 | 15.137255938093 | 5.71661900396787 | 1.7138338128214 | 0.759359030414403 | 0.351110200278079 |
| TCF4 | 5.59648314409974 | 0.149982882476814 | 0.280889508455173 | 1.43388340709661 | 6.96146309463079 |
| TCL1A | 0.0513603538737202 | 0 | 0.175558462420334 | 0.129041215983472 | 0.100432568101733 |
| VPREB3 | 0 | 0.0983907274710984 | 0.33391866565921 | 0.51018118066222 | 0.0789663494322397 |
| ACVRL1 | 0.428451188244593 | 0.129862974505345 | 0.267069845236772 | 0.666626998397525 | 1.09234822504098 |
| ADAMDEC1 | 0 | 0 | 0 | 0 | 0.236849813770509 |
| ALDH1A2 | 0 | 0 | 0 | 0.117068089614854 | 0.0458657199625384 |
| CCRL2 | 2.04129007638552 | 0.957673807099835 | 2.51730027777864 | 3.23743057641214 | 5.44466918719567 |
| CD86 | 0.0603402220417124 | 0.0760981608177539 | 0.188536589959567 | 0.0865791200124716 | 140.844033128846 |
| CFB | 0.189463391716347 | 0.298829768647172 | 0.202456383752594 | 7.22225726135961 | 3.19652157534609 |
| CXCL2 | 0.347510797495381 | 0.0690572869185464 | 0.184183175055644 | 0.767574713260437 | 7.1025906041116 |
| CXCL3 | 0.0764373381946375 | 0 | 0 | 0 | 2.22007793902901 |
| EREG | 4.13074188511085 | 0.872463173445688 | 1.21352611175253 | 4.60632409168193 | 3.53114879594062 |
| ICAM1 | 26.3225806061086 | 3.69508010525222 | 11.5612613899165 | 15.986869569612 | 36.1322728223818 |
| INHBA | 0.20000418280766 | 0 | 0.035747276231363 | 0.477624035862211 | 0.178770378218121 |
| LILRB4 | 0.725275080431343 | 0.0490182458691303 | 0.181584515477741 | 0.520041555026167 | 106.635462765275 |
| NUPR1 | 0 | 0 | 0 | 0.709012158561964 | 0 |
| PDPN | 0.63459380914047 | 0.162712173711314 | 0.202613723750939 | 0.939531337631743 | 0.347168317234732 |
| SERPING1 | 0.305133884924488 | 0 | 0 | 0.754461919408032 | 5.42399696368157 |
| SLC1A3 | 0 | 0 | 0 | 0 | 0.390851817530305 |
| SLC6A12 | 0.125382937018582 | 0 | 0.0596110059041317 | 0.489478964920557 | 9.90369164253591 |
| SQOR | 33.4404396709447 | 35.7465898070398 | 29.1984454186279 | 31.9313822498785 | 72.7178231004998 |
| VSIG4 | 0.25378754292522 | 0 | 0.286373784590677 | 0 | 5.16089841053076 |
| ACE | 3.16914580353855 | 8.97933308798429 | 6.66565749227824 | 5.13496022293496 | 8.62409948698236 |
| CISH | 19.1603796488508 | 15.6036929723125 | 11.7567208822068 | 4.26216593973645 | 7.74760517731118 |
| CLEC4A | 4.81850929843966 | 1.394315641778 | 1.4271109997333 | 3.23483676986554 | 89.0256998837511 |
| CLIC2 | 1.22132171269956 | 0.541267300470522 | 0.875651677000239 | 0.705357946746145 | 24.2207647644875 |
| COLEC12 | 0 | 0 | 0 | 0.0574930656175801 | 0 |
| ECM1 | 0.705727159671569 | 0.348000312233568 | 0.648906151183534 | 0.707579953444764 | 1.0856385983402 |
| ENPP2 | 0.660793376333027 | 1.08359402245564 | 0.601269588041625 | 0.123443624549889 | 1.43377461303016 |
| GATM | 0.800575597823844 | 0.0631246455604276 | 0.229462317519857 | 0.128905895618398 | 2.9684412010666 |
| GFRA2 | 0.465446244341031 | 0.258413822048901 | 0.0508338090906309 | 0.334103930283533 | 4.77493936405185 |
| IL1R1 | 1.88667111727635 | 1.52438865417414 | 0.386687176973508 | 5.53024419991008 | 2.70708875128472 |
| MAOA | 0 | 0.0125032470379778 | 0.0454643403057873 | 0.0391885031842564 | 0.0178614401119112 |
| MS4A4A | 0.654922083659694 | 0 | 0 | 0 | 62.824517858648 |
| PLTP | 5.76781005065948 | 0.193552235262807 | 0.725212589421827 | 1.31464816885043 | 4.57423452841977 |
| PTGS1 | 2.02625582522416 | 0.031677643711617 | 0.159772820629484 | 0.221839405913356 | 38.5115861058714 |
| RNASE6 | 2.57034224061085 | 1.55413621826526 | 0.755928891264297 | 0 | 62.6203374489481 |
| SLC7A8 | 0.310334431394085 | 0.832472050906884 | 1.83434770463846 | 0.608183780856691 | 0.363059196213789 |
| TREM2 | 0 | 0 | 0 | 0.293445749875763 | 0 |
| WNT5B | 0.0257615286791872 | 0 | 0 | 0.865764388560015 | 0.314581209761574 |
| CD1D | 0.252652767110177 | 0.247373650540447 | 0.202885755820835 | 0.359075844200055 | 67.5964214961601 |
| CD36 | 1.07824712179783 | 0.339264584628864 | 0.539586113154075 | 1.02237593338146 | 58.8810398010101 |
| CSTA | 2.87646723397113 | 0 | 0.13398122880916 | 0 | 173.545483717939 |
| FXYD6 | 0 | 0 | 0 | 0.139995512052629 | 1.33426263346427 |
| GAS7 | 61.2730926695054 | 1.09417281111962 | 8.15823083234582 | 1.53088598986978 | 36.2498743535205 |
| HPSE | 1.96424981646418 | 6.47007322735653 | 3.56747341418466 | 4.89595787663339 | 4.98911096611645 |
| KBTBD11 | 0.678693638810208 | 4.48511612182804 | 2.03245735399911 | 3.04382537681445 | 6.87417954693535 |
| NRG1 | 0 | 0 | 0 | 0.242223160741488 | 0.814593946439049 |
| NRGN | 1.25471218405089 | 0.912555499818482 | 1.15621215124602 | 0.303468410517509 | 105.282501129573 |
| SMARCD3 | 6.81727686861426 | 13.8066611865776 | 22.8722104255595 | 4.58467417413573 | 20.9336784365503 |
| TMEM170B | 2.95644229394717 | 1.5514674206198 | 1.9499492717142 | 1.40404641978291 | 30.7360994681166 |
| ABHD5 | 6.90492928218358 | 2.47045921352886 | 2.95982783294023 | 6.42374466812199 | 9.24758721802321 |
| ADGRG3 | 18.2908053036202 | 0.0733607986856278 | 0.615943843013934 | 0.376632585531813 | 0.305176850952508 |
| BTNL8 | 0.479883817241601 | 0.158883147138341 | 0.228664581398865 | 0.389246030903637 | 0.691525632497091 |
| C5AR2 | 3.38768793167968 | 0.962839427590935 | 0.73119536953304 | 0.473999219297012 | 66.1731666799966 |
| CEACAM1 | 4.66892245154762 | 5.68918318884606 | 2.80889457471759 | 7.14074535916833 | 12.0611433565834 |
| CEP19 | 5.06669234801712 | 2.66570814891806 | 2.48624336661689 | 4.24522908721427 | 6.17874086299019 |
| CYP4F3 | 0.105992781145326 | 0 | 0 | 0.199876431121254 | 0.226025182785768 |
| DAPK2 | 6.80556737844844 | 1.3791998358232 | 3.26502596754617 | 2.77397194707662 | 1.97127899697657 |
| DGAT2 | 0.284534171295842 | 0.105764334237416 | 0.199429446468928 | 0.186774693982638 | 4.24676158538125 |
| DOCK5 | 13.9873115430715 | 0.315693155901236 | 0.373828744848556 | 0.934168567795143 | 83.283907122057 |
| DPEP3 | 1.98268277650309 | 1.0559804494746 | 1.33494451788636 | 1.09473669328588 | 2.3776573969495 |
| DYSF | 11.3840128235166 | 0.103514194434563 | 4.16142977523703 | 0.212271150442656 | 3.4868079835584 |
| FPR2 | 0.68117408211071 | 0.0513704529427299 | 0.0289443713323537 | 0.116065287115604 | 32.3578631768778 |
| HAL | 0.962453110710969 | 0.30453339144366 | 0.407760585705102 | 1.24037983200944 | 28.8946093503189 |
| KCNJ15 | 0.148403005246877 | 0.0657015475775988 | 0.0247869751659843 | 0 | 0.0382922964239043 |
| LILRA2 | 5.57694984155128 | 0.0881702437305924 | 0.243810914640281 | 0.524038294484574 | 287.269575017517 |
| LRRC4 | 1.06878195792169 | 0.0992332115370332 | 0.495925422318774 | 0.102350769617004 | 9.64418514529133 |
| LRRK2 | 1.11380161270323 | 0.0569489132430742 | 0.100808478019541 | 0.0593413615903732 | 78.8687739177303 |
| LY96 | 1.99067468660042 | 13.1515187442418 | 3.85085911801567 | 13.9658486500062 | 80.9840356935864 |
| MGAM | 14.6226160451376 | 0.0361756814742134 | 0.135863299590202 | 0.0314131507771003 | 0.0673647449302766 |
| MME | 0.0909303358144165 | 0.113295410234375 | 0.102228493326538 | 0.108821895225172 | 0.0153137845096485 |
| NINJ2 | 12.8232872441608 | 16.6698023408072 | 10.3239171759153 | 15.5366175015208 | 18.5527363188164 |
| PPP1R3B | 4.98270731759646 | 5.757282494823 | 4.21500806076263 | 14.0605672995018 | 2.69720231995991 |
| REM2 | 8.71505768885506 | 7.91170273671624 | 5.30907305650755 | 45.0947045269146 | 1.27822351668252 |
| REPS2 | 0.992161337730061 | 0.505194326190745 | 0.335317354777063 | 0.656181780537616 | 4.89323694665316 |
| TECPR2 | 7.94703596204946 | 5.78848140424983 | 5.6323898018688 | 4.01582252415688 | 8.04953732644056 |
| TLR1 | 17.7405479712491 | 7.94611327530388 | 4.78537608080753 | 4.79914225017346 | 64.7769386499769 |
| TLR4 | 2.21835206652253 | 0.0357914339941007 | 0.118066786501105 | 0.0353011953473687 | 66.0643019497426 |
| TMCC3 | 14.534413642262 | 0.346112750767616 | 1.85519257378848 | 0.243206172614697 | 9.19131828004531 |
| TMEM154 | 4.15028940832051 | 9.54713669003299 | 5.65799253836837 | 10.4241579849978 | 51.6538185293819 |
| ABCB1 | 35.6508877086871 | 2.11898303280204 | 9.28339632057 | 0.454617179592002 | 0.082439291645955 |
| AUTS2 | 66.7609172040971 | 11.1075589050532 | 14.2835799205952 | 1.58177171037035 | 1.00056493747498 |
| COLGALT2 | 16.9619578121379 | 0.240984142843278 | 1.28494809224896 | 0.139243037830348 | 0.234177907775057 |
| EOMES | 34.7156343600852 | 1.30604735192787 | 14.3770029468759 | 0.151428593146513 | 0.25216006042113 |
| FASLG | 40.8492412586392 | 0.623718027569546 | 2.98019012348235 | 0.71212345904445 | 0.441441156866181 |
| GK5 | 133.46318029013 | 12.3841164304741 | 21.0728335858943 | 11.9032358614986 | 21.6937690965472 |
| GNG2 | 347.443875680483 | 83.4073676703523 | 94.1001706756385 | 87.8178091927617 | 141.304391970948 |
| HOPX | 468.478658307824 | 15.5377357384362 | 57.4276028639375 | 1.61089698719135 | 5.8914750536465 |
| KLRD1 | 246.290655037427 | 2.02126613443466 | 20.1772202718613 | 4.63185207418001 | 4.43383028233963 |
| MATK | 530.636754170876 | 11.0693074974601 | 106.17025207134 | 1.37217485670109 | 1.96233669673698 |
| MLC1 | 113.094524217669 | 0.1296925507579 | 1.72942783244647 | 0.311566324010531 | 22.4326964772497 |
| NCALD | 47.2777278774844 | 4.73280850210663 | 11.2361172230354 | 3.31577762610745 | 0.718316718859927 |
| NLRC3 | 173.707612366465 | 67.8169579284791 | 81.097680777408 | 81.1537468189753 | 1.38381225093569 |
| PDGFD | 42.3123568410012 | 0.0617097282978706 | 4.08244985369889 | 0 | 0.149292404546203 |
| PLEKHF | 136.220494481129 | 19.7601735328878 | 36.3875036714117 | 19.4829374061383 | 0.35839486365219 |
| PPP2R2B | 39.0032550123116 | 3.51475258905942 | 13.5026972043889 | 3.21225579522072 | 0.253709164352637 |
| PYHIN1 | 261.730478134421 | 40.2737734165784 | 55.2585693696872 | 67.9548105795143 | 0.774951434334451 |
| RNF165 | 35.5434340680214 | 0.117728247923624 | 0.887761088278177 | 0.252740042233168 | 0.081893802886078 |
| S1PR5 | 461.622655883738 | 1.73149267288106 | 41.7720941125901 | 0.529389171769584 | 0.941674082801863 |
| SAMD3 | 367.321483262895 | 57.8665949411827 | 120.297178579186 | 19.9764968089219 | 2.28183641457969 |
| SBK1 | 66.5865700183104 | 16.6923511976197 | 27.4766211450453 | 11.0229110496692 | 0.878348789486539 |
| SH2D2A | 318.736817568831 | 36.7051553439723 | 66.4500413369875 | 28.9077019899112 | 0.586233070168583 |
| SLFN13 | 81.3551353936778 | 9.88957363429813 | 14.6940935708963 | 7.71790489678105 | 1.80270414062867 |
| STAT4 | 256.875614024983 | 73.2041216492301 | 123.022378087081 | 80.1857788989678 | 3.16596535100721 |
| TOGARAM2 | 62.6412276380914 | 1.93892670476424 | 5.95616893994568 | 6.08552759746374 | 0.979826510624534 |
| TSPOAP1 | 298.272861769601 | 11.825345994764 | 32.5275419110664 | 56.2183401041487 | 5.95571687968183 |
| TTC38 | 325.703098369836 | 4.25650122194311 | 21.6000422452249 | 3.29072865422217 | 13.9440199161427 |
| TXK | 289.240921035468 | 71.0453406251931 | 82.3552864784903 | 41.5394225337838 | 3.26555028659687 |
| YPEL1 | 105.166800704502 | 5.96877974833461 | 14.8377005837951 | 3.98226370462469 | 4.40046034156515 |
| ZBTB16 | 86.352627233819 | 4.85074087899058 | 12.1416121787729 | 2.9900499195532 | 7.3411866820438 |
| FHIT | 6.12221931010403 | 98.2539901326223 | 17.2088952793353 | 11.9337879122037 | 1.98969687632555 |
| TRAT1 | 1.82527686234142 | 116.662055071454 | 42.5594408230054 | 45.8588697175739 | 0.0669900605780553 |
| TSHZ2 | 0.077094463755214 | 10.3402256706886 | 0.530722197270164 | 6.07833167161168 | 0.0355205210753674 |
| TSPAN18 | 2.76885689467624 | 50.120140979035 | 14.0372256147086 | 4.73324441237652 | 0.434106047809428 |
| WNT7A | 0 | 17.0883963820579 | 4.57635871659645 | 0.606321578184723 | 0 |
| CD248 | 0.0217616773367871 | 1.50652820945721 | 16.7131892385614 | 0.477844626725084 | 0 |
| CD8A | 5.16732843080116 | 1.99249727130834 | 538.216797539064 | 1.22352364938439 | 0.218412818059309 |
| CD8B | 5.63372501931945 | 4.94154824531243 | 690.196951723652 | 6.55912029872283 | 2.72207773905785 |
| FCGBP | 4.87186057602238 | 2.60228020137674 | 13.8271423408746 | 1.01047909707915 | 1.15535396150461 |
| CCR4 | 0.0347657920943443 | 58.0676088483504 | 7.43907327707358 | 150.751192708116 | 0 |
| CD5 | 0.8921825570264 | 193.010470187532 | 130.797307511882 | 317.912181219475 | 4.30120434495132 |
| CTLA4 | 0.46586245398606 | 49.5392798571527 | 8.40674447708265 | 325.121345120445 | 0.362548596458703 |
| FAM160B1 | 27.6647241983289 | 15.3767834408427 | 18.7547777958565 | 86.397493390073 | 8.53151465501255 |
| FAM46C | 105.655349650099 | 95.0297332666513 | 98.5188929497421 | 276.884033768097 | 0.153696326240277 |
| FOXP3 | 0.93322145283811 | 24.9789912227117 | 1.75647197347746 | 313.685821002479 | 0.816092788714517 |
| GCNT4 | 0.521541094717927 | 13.5084629276821 | 6.37936890233892 | 27.0767641549312 | 0.299027573535988 |
| HPGD | 8.86691928763713 | 5.43663408188655 | 8.74481612071862 | 94.5114858522998 | 0.331635152719544 |
| ICOS | 0.961526295821154 | 115.112148735985 | 36.1142232310724 | 260.934371434011 | 0.15562837480452 |
| LAYN | 0.920713691127399 | 0.495056559308106 | 1.516722992923 | 23.3669409859553 | 2.52077384568442 |
| LRRC32 | 0 | 1.26875800849278 | 0.0558000204903282 | 26.8628640411678 | 0.106702947830908 |
| PLCL1 | 0.361016216391006 | 16.9077427846048 | 5.58950922758938 | 46.2798126734051 | 2.13598032853746 |
| RTKN2 | 3.23745014567294 | 13.3831296624276 | 4.52248414125275 | 105.39655850358 | 2.07597260602569 |
| SEMA3G | 0.0115186632413928 | 1.36963688321624 | 0.0837724540300752 | 13.6866231859119 | 0 |
| AATBC | 0.862381185603521 | 0.169612370993582 | 0.395075723327132 | 1.51582707226835 | 122.670067089138 |
| FZD1 | 0.0909306042602444 | 0.206749189436629 | 0 | 0.0431761999161938 | 47.5719235812834 |
| GRK3 | 1.68403817993922 | 1.35750453706955 | 1.28712027060365 | 4.64167585685216 | 116.14265581675 |
| GUCY1A3 | 4.36278835223315 | 0 | 0.0290212664133315 | 0.125262148775337 | 17.560921932425 |
| KDM1B | 9.94998918107382 | 6.19604745256449 | 5.86026195214282 | 5.63208170344591 | 144.194033835313 |
| LMO2 | 1.63912146426352 | 0 | 0 | 0.242979350174535 | 298.59719763019 |
| RNF144B | 1.46579176099642 | 1.63738885565927 | 1.30452647836947 | 0.891397177214894 | 151.249057000754 |
| SLC2A6 | 14.0815583690715 | 1.95998168347342 | 8.79203361079547 | 3.46983634170838 | 315.921537472941 |
| TTYH2 | 5.02131345974304 | 11.367234453737 | 6.97316248163613 | 6.566632857657 | 107.498219237942 |

In some instances, nucleic acid expression levels of the genes described herein may be measured by polymerase chain reaction (PCR)-based assays, e.g., quantitative PCR, real-time PCR, quantitative real-time PCR (qRT-PCR), reverse transcriptase PCR (RT-PCR), and reverse transcriptase quantitative PCR (RT-qPCR). Platforms for performing quantitative PCR assays include Fluidigm (e.g., BIOMARK™ HD System). Other amplification-based methods include, for example, transcript-mediated amplification (TMA), strand displacement amplification (SDA), nucleic acid sequence based amplification (NASBA), and signal amplification methods such as bDNA.

In some instances, nucleic acid expression levels of the genes described herein also may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene.

Primers and probes may be labeled with a detectable marker, such as, for example, a radioisotope, fluorescent compound, bioluminescent compound, a chemiluminescent compound, metal chelator, or enzyme. Such probes and primers can be used to detect the presence of expressed genes (e.g., the genes described herein) in a sample. As will be understood by the skilled artisan, many different primers and probes may be prepared based on the sequences provided herein (or, in the case of genomic DNA, their adjacent sequences) and used effectively to amplify, clone, and/or determine the presence and/or expression levels of the genes described herein.

Other methods to detect nucleic acid expression levels of the genes described herein include electrophoresis, Northern and Southern blot analyses, in situ hybridization (e.g., single or multiplex nucleic acid in situ hybridization), RNAse protection assays, and microarrays (e.g., Illumina BEADARRAY™ technology; Beads Array for Detection of Gene Expression (BADGE)).

In some instances, the macrophage biomarker can be analyzed by a number of methodologies, including, but not limited to, RNA-seq, PCR, RT-qPCR, qPCR, multiplex qPCR, multiplex RT-qPCR, NANOSTRING® nCOUNTER® Gene Expression Assay, microarray analysis, serial analysis of gene expression (SAGE), Northern blot analysis, MassARRAY, ISH, whole genome sequencing, FACS, spatial transcriptomics, spatial proteomics, Western blot, ELISA, immunoprecipitation, immunohistochemistry, immunofluorescence, radioimmunoassay, dot blotting, immunodetection methods, surface plasmon resonance, optical spectroscopy, mass spectrometery, and HPLC, or combinations thereof.

### (ii) RT-qPCR

In some instances, nucleic acid expression levels of the genes described herein (e.g., genes in a M1 macrophage gene signature set or genes in a gene signature matrix) can be detected using reverse transcription quantitative polymerase chain reaction (RT-qPCR). The technique of RT-qPCR is a form of PCR wherein the nucleic acid to be amplified is RNA that is first reverse transcribed into cDNA and the amount of PCR product is measured at each step in a PCR reaction. As RNA cannot serve as a template for PCR, the first step in gene expression profiling by PCR is the reverse transcription of the RNA template into cDNA, followed by its amplification in a PCR reaction. For example, reverse transcriptases may include avilo myeloblastosis virus reverse transcriptase (AMY-RT) or Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMPTM RNA PCR kit (Perkin Elmer, Calif, USA), following the manufacturer's instructions. The derived cDNA can then be used as a template in the subsequent PCR reaction.

A variation of the PCR technique is quantitative real time PCR (qRT-PCR), which measures PCR product accumulation through a dual-labeled fluorigenic probe (i.e., TAQMAN® probe). The technique of quantitative real time polymerase chain reaction refers to a form of PCR wherein the amount of PCR product is measured at each step in a PCR reaction. This technique has been described in various publications including Cronin et al., Am. J. Pathol. 164W: 35-42 (2004); and Ma et al., Cancer Cell 5: 607-616 (2004). Real time PCR is compatible both with quantitative competitive PCR, where an internal competitor for each target sequence is used for normalization, and/or with quantitative comparative PCR using a normalization gene contained within the sample, or a housekeeping gene for PCR. For further details see, e.g., Held et al., Genome Research 6: 986-994 (1996).

The steps of a representative protocol for profiling gene expression using fixed, paraffin- embedded tissues as the RNA source, including mRNA isolation, purification, primer extension and amplification are given in various published journal articles (for example: Godfrey et al., Malec. Diagnostics 2: 84-91 (2000); Specht et al., Am. J. Pathol. 158: 419-29 (2001)). Briefly, a representative process starts with cutting a section (e.g., a 10 microgram section) of a paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

The nucleic acid expression level determined by an amplification-based method (e.g., RT-qPCR) may be expressed as a cycle threshold value (Ct). From this value, a normalized expression level for each gene can be determined, e.g., using the delta Ct (dCt) method as follows: Ct(Control/Reference Gene)−Ct (Gene of Interest/Target Gene)=dCt (Gene of Interest/Target Gene). One of skill in the art will appreciate that the dCt value obtained may be a negative dCt value or a positive dCt value. As defined herein, a higher dCt value indicates a higher expression level of the gene of interest relative to the control gene. Conversely, a lower dCt value indicates a lower expression level of the gene of interest relative to the control gene. In cases where the expression levels of a plurality of genes has been determined, the expression level for each gene, e.g., expressed as a dCt value, may then be used to determine a single value that represents an aggregate or composite expression level for the plurality of genes (e.g., genes in a M1 macrophage gene signature set). The aggregate or composite expression level may be the mean or median of dCt values determined for each target gene/gene of interest. As defined herein, a higher averaged dCt or median dCt value indicates a higher aggregative expression level of the plurality of target genes relative to the control gene (or plurality of control genes). A lower averaged dCt or median dCt value indicates a lower aggregative expression level of the plurality of target genes relative to the control gene (or plurality of control genes). Expression levels may be compared to a reference level.

In one particular instance, the nucleic acid expression levels described herein may be determined using a method including:

(a) obtaining or providing a sample from the individual, wherein the sample includes a tumor tissue sample (e.g., a paraffin-embedded, formalin-fixed tumor tissue sample);

(b) isolating mRNA from said sample;

(c) performing reverse transcription of the mRNA into cDNA (e.g., for at least one of the genes described herein (e.g., genes in a M1 macrophage gene signature set or genes in a gene signature matrix));

(d) amplifying the cDNA (e.g., for at least one of the genes described herein (e.g., genes in a M1 macrophage gene signature set or genes in a gene signature matrix)) using PCR; and

(e) quantifying the nucleic acid expression levels (e.g., for at least one of the genes described herein (e.g., genes in a M1 macrophage gene signature set or genes in a gene signature matrix)).

One or more genes (e.g., one, two, three, four, five, six, seven, eight, nine, ten or more genes (e.g., 55, 82, 89, 106, 153, or 170 genes)) may be detected in a single assay depending on the primers or probes used. Further, the assay may be performed across one or more tubes (e.g., one, two, three, four, five, six, seven, eight, nine, ten or more tubes (e.g., 55, 82, 89, 106, 153, or 170 tubes)).

In some instances, the method further comprises (f) normalizing the nucleic acid expression level of the gene(s) (e.g., at least one of the genes described herein (e.g., genes in a M1 macrophage gene signature set or genes in a gene signature matrix)) in said sample to the expression level of one or more reference genes (e.g., one, two, three, four, five, six, seven, eight, nine, or more reference genes, e.g., a housekeeping gene (e.g., β-actin)). For example, RT-qPCR may be used to analyze the expression level of the genes described herein ((e.g., at least one of the genes described herein (e.g., genes in a M1 macrophage gene signature set or genes in a gene signature matrix)) to generate an expression level that reflects a normalized, averaged dCT value for the analyzed genes.

### (iii) RNA-Seq and Microarray

In some instances, nucleic acid expression levels of the genes described herein (e.g., genes in a M1 macrophage gene signature set or genes in a gene signature matrix) can be detected using RNA-seq. RNA-seq, also called Whole Transcriptome Shotgun Sequencing (WTSS), refers to the use of high-throughput sequencing technologies to sequence and/or quantify cDNA in order to obtain information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009). In some instances, sequencing quality control is performed. In some instances, counts are normalized to transcripts per million (TPM).

### (a) Marker Gene Approaches

Marker gene approaches use the expression of one or more genes within a gene signature set (see, e.g., Table 1 and Table 2) to determine a macrophage biomarker (e.g., a number of M1 macrophages in a sample). In some instances, the marker gene approach uses xCell (see, e.g., Aran et al. Genome Biol. 18(1): 220 (2017)).

The gene signature sets exemplified in Table 1 may be modified to remove, substitute, or add genes. In some instances, the number of genes in any of the exemplified gene signature sets can be increased or reduced by one or more genes (e.g., one, two, three, four, five, six, seven, eight, nine, ten, or more genes). In some instances, the number of genes in any of the exemplified gene signature sets can be increased or reduced by between about 5% and about 20% (e.g., 5%, 6%, 7%, 8%, 9%, 10%, 11%, 12%, 13%, 14%, 15%, 16%, 17%, 18%, 19%, or 20%). In some instances, genes from any of the exemplified gene signature sets can be substituted with genes in the same signaling pathway. In some instances, genes from any of the exemplified gene signature sets can be added to a different exemplified gene signature set (e.g., a gene from gene signature set 1 (e.g., C1QA) can be added to gene signature set 2) to generate a gene signature set. In some instances, a gene signature set can comprise genes that are present in all exemplified gene signature sets (i.e., ACP2 and ADAMDEC1) or most exemplified gene signature sets (e.g., FDX1, CD163, HAMP, ABCD1, C1QA, CCL22, and TREM2). In some instances, a gene signature set comprises ACP2 and ADAMDEC1. In some instances, a gene signature set comprises ACP2, ADAMDEC1, and FDX1. In some instances, a gene signature set comprises ACP2, ADAMDEC1, and CD163. In some instances, a gene signature set comprises ACP2, ADAMDEC1, and HAMP. In some instances, a gene signature set comprises ACP2, ADAMDEC1, FDX1, and CD163. In some instances, a gene signature set comprises ACP2, ADAMDEC1, FDX1, and HAMP. In some instances, a gene signature set comprises ACP2, ADAMDEC1, CD163, and HAMP. In some instances, a gene signature set comprises ACP2, ADAMDEC1, FDX1, CD163, and HAMP. In some instances, a gene signature set comprises ACP2, ADAMDEC1, FDX1, CD163, HAMP, and ABCD1. In some instances, a gene signature set comprises ACP2, ADAMDEC1, FDX1, CD163, HAMP, and CCL22. In some instances, a gene signature set comprises ACP2, ADAMDEC1, FDX1, CD163, HAMP, and C1QA. In some instances, a gene signature set comprises ACP2, ADAMDEC1, FDX1, CD163, HAMP, and TREM2. In some instances, a gene signature set comprises ACP2, ADAMDEC1, FDX1, CD163, HAMP, ABCD1, and CCL22.

The gene signature sets exemplified in Table 2 may be modified to remove, substitute, or add genes. In some instances, the number of genes in any of the exemplified gene signature sets can be increased or reduced by one or more genes (e.g., one, two, three, four, five, six, seven, eight, nine, ten, or more genes). In some instances, the number of genes in any of the exemplified gene signature sets can be increased or reduced by between about 5% and about 20% (e.g., 5%, 6%, 7%, 8%, 9%, 10%, 11%, 12%, 13%, 14%, 15%, 16%, 17%, 18%, 19%, or 20%). In some instances, genes from any of the exemplified gene signature sets can be substituted with genes in the same signaling pathway. In some instances, genes from any of the exemplified gene signature sets can be added to a different exemplified gene signature set (e.g., a gene from gene signature set 1 (e.g., CD48) can be added to gene signature set 4) to generate a gene signature set. In some instances, a gene signature set can comprise genes that are present in most exemplified gene signature sets (e.g., ACP2, FDX1, HK3, MSR1, CD84, SDS, VSIG4, CLECSA, ADAMDEC1, HAMP, DNASE2B, and CYBB). In some instances, a gene signature set comprises ACP2. In some instances, a gene signature set comprises FDX1. In some instances, a gene signature set comprises HK3. In some instances, a gene signature set comprises MSR1. In some instances, a gene signature set comprises CD84. In some instances, a gene signature set comprises SDS. In some instances, a gene signature set comprises VSIG4. In some instances, a gene signature set comprises CLECSA. In some instances, a gene signature set comprises ADAMDEC1. In some instances, a gene signature set comprises HAMP. In some instances, a gene signature set comprises DNASE2B. In some instances, a gene signature set comprises CYBB. In some instances, a gene signature set comprises 1, 2, 3, 4, 5, 6, 7, 8, 9, 10, or 11 of ACP2, FDX1, HK3, MSR1, CD84, SDS, VSIG4, CLECSA, ADAMDEC1, HAMP, DNASE2B, and CYBB. In some instances, a gene signature set comprises ACP2, FDX1, HK3, MSR1, CD84, SDS, VSIG4, CLECSA, ADAMDEC1, HAMP, DNASE2B, and CYBB.

The steps of a representative protocol for determining a number of macrophages (e.g., M1 macrophages) using an RNA-seq marker gene approach can be found in Aran et al. Genome Biol. 18(1):220 (2017). Briefly, sequence scores (e.g., pre-processed raw sequence reads) obtained from a sample are transformed based on a power function derived from synthetic mixtures of a cell type (e.g., M1 macrophages) and a control cell type (e.g., multipotent progenitor cells or endothelial cells) within a range (e.g., 0.8% to 25.6%) based on expected abundance of the cell type present in the sample. The transformed scores are further adjusted using a spillover compensation matrix (limited to 0.5 off the diagonal) derived from synthetic mixtures of 25% of a cell type (e.g., M1 macrophages) and 75% of a control cell type (e.g., multipotent progenitor cells or endothelial cells). The final adjusted score represents the fraction of the cell type present in the sample.

### (b) Deconvolution Approaches

Deconvolution approaches use the expression of one or more genes within a gene signature matrix (see, e.g., Table 3 and Table 4) to determine a macrophage biomarker (e.g., a number of M1 macrophages in a sample). In some instances, the deconvolution approach uses quanTlseq (see, e.g., Finotello et al. Genome Med. 11(1): 34 (2019)).

The steps of a representative protocol for determining a number of M1 macrophages using an RNA-seq deconvolution approach can be found in Finotello et al. Genome Med. 11(1): 34 (2019). Briefly, sequence scores (e.g., pre-processed raw sequence reads) obtained from a sample are normalized and deconvoluted 25 using a gene signature matrix (see, e.g., Table 3 and Table 4) to calculate proportions of cell types (e.g., M1 macrophages, M2 macrophages, B cells, monocytes, neutrophils, NK cells, non-regulatory CD4⁺ T cells, CD8⁺ T cells, regulatory T cells, dendritic cells, or other cell types) present in the sample using constrained least squares regression.

### (iv) Immunohistochemistry

In some instances, macrophages (e.g., M1 macrophages) can be detected using immunohistochemistry (IHC). In some instances, any of the genes described herein (e.g., CD68, genes in a M1 macrophage gene signature set, or genes in a gene signature matrix) may be used to identify macrophages (e.g., M1 macrophages) or distinguish macrophages from other cell types. In some instances, an antibody specific for any of the genes described herein (e.g., CD68, genes in a M1 macrophage gene signature set, or genes in a gene signature matrix) is used as a primary antibody in the IHC assay. In some instances, a horseradish peroxidase (HRP)-conjugated secondary antibody is used in the IHC assay. In some instances, a signal from the IHC assay is compared to an IHC assay performed with a negative control antibody. In some instances, macrophages (e.g., M1 macrophages) can be detected using an antibody that binds CD68.

### (v) Flow Cytometry

In some instances, macrophages (e.g., M1 macrophages) can be detected using flow cytometry. In some instances, any of the genes described herein (e.g., genes in a M1 macrophage gene signature set or genes in a gene signature matrix) may be used to identify macrophages (e.g., M1 macrophages) or distinguish macrophages from other cell types. In some instances, an antibody specific for any of the genes described herein (e.g., genes in a M1 macrophage gene signature set or genes in a gene signature matrix) is used to label macrophages (e.g., M1 macrophages).

### (vi) Samples

The sample may be taken from an individual who is suspected of having, or is diagnosed as having, a lymphoma, and hence is likely in need of treatment, or from a healthy individual who is not suspected of having a lymphoma or who does not have lymphoma but has a family history of a lymphoma. For assessment of gene expression, samples, such as those containing cells, or proteins or nucleic acids produced by these cells, may be used in the methods of the present invention. The expression level of a gene can be determined by assessing the amount (e.g., the absolute amount or concentration) of the markers in a sample (e.g., a tissue sample, e.g., a tumor tissue sample, such as a biopsy). In addition, the level of a gene can be assessed in bodily fluids or excretions containing detectable levels of genes. Bodily fluids or secretions useful as samples in the present invention include, e.g., blood, urine, saliva, stool, pleural fluid, lymphatic fluid, sputum, ascites, prostatic fluid, cerebrospinal fluid (CSF), or any other bodily secretion or derivative thereof. The word blood is meant to include whole blood, plasma, serum, or any derivative of blood. Assessment of a gene in such bodily fluids or excretions can sometimes be preferred in circumstances where an invasive sampling method is inappropriate or inconvenient. In other embodiments, a tumor tissue sample is preferred.

The sample may be frozen, fresh, fixed (e.g., formalin fixed), centrifuged, and/or embedded (e.g., paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation, such as formalin fixation.

In one particular instance, the sample is a clinical sample. In another instance, the sample is used in a diagnostic assay, such as a diagnostic assay or diagnostic method of the invention. In some instances, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For example, samples of lymphoma lesions may be obtained by resection, fine needle aspiration, pleural fluid, or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In some instances, the sample from the individual is a tissue sample, a whole blood sample, a plasma sample, a serum sample, or a combination thereof. In some instances, the sample is a tissue sample. In some instances, the sample is a tumor tissue sample. In some instances, the sample is obtained prior to treatment. In some instances, the tissue sample is formalin-fixed and paraffin-embedded (FFPE) sample, an archival sample, a fresh sample, or a frozen sample. In some instances, the sample from the individual is a tissue sample. In some instances, the tissue sample is a tumor tissue sample (e.g., biopsy tissue). In some instances, the tumor tissue sample includes tumor cells, tumor infiltrating immune cells, stromal cells, normal adjacent tissue (NAT) cells, or a combination thereof. In some instances, the tissue sample is a biopsy. In some instances, the tissue sample is blood cells, lymph nodes, or bone/bone marrow.

In some instances, the tumor tissue sample is extracted from a malignant cancerous tumor (i.e., cancer). In some instances, the cancer is a solid tumor, or a non-solid or soft tissue tumor. In some instances, the tumor tissue sample is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma)) sample.

### (vii) RNA Extraction

Prior to detecting the level of a nucleic acid, mRNA may be isolated from a target sample. In some instances, the mRNA is total RNA isolated from tumors or tumor cell lines or, alternatively, normal tissues or cell lines. If the source of mRNA is a primary tumor, mRNA can be extracted, for example, from frozen or archived paraffin-embedded and fixed (e.g., formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56: A67 (1987), and De Andres et al., Bio Techniques 18: 42044 (1995). In particular, RNA isolation can be performed using a purification kit, buffer set, and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated, for example, by using RNA Stat-60 (TelTest). RNA prepared from tumor tissue samples can also be isolated, for example, by cesium chloride density gradient centrifugation.

### (viii) Expression Level

The expression level may reflect the expression levels of one or more genes described herein (e.g., one or more genes in a M1 macrophage gene signature set or one or more genes in a gene signature matrix). In certain instances, the detected expression level of each gene is normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for both differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping gene (e.g., β-actin)). For example, in some instances, the nucleic acid expression levels detected using the methods described herein (e.g., for at least one of the genes described herein (e.g., genes in a M1 macrophage gene signature set or genes in a gene signature matrix)) may be normalized to the expression level of one or more reference genes (e.g., one, two, three, four, five, six, seven, eight, nine, or more reference genes, e.g., a housekeeping gene (e.g., β-actin)). Alternatively, normalization can be based on the average signal or median signal of all of the assayed genes. On a gene-by-gene basis, a measured normalized amount of an mRNA can be compared to the amount found in a reference expression level. The presence and/or expression level/amount measured in a particular subject sample to be analyzed will fall at some percentile within this range, which can be determined by methods well known in the art.

In other instances, to determine an expression level, the detected expression level of each assayed gene is not normalized.

The expression level may reflect the aggregate or composite expression level of a single gene or a plurality of genes described herein (e.g., for at least one of the genes described herein (e.g., genes in a M1 macrophage gene signature set or genes in a gene signature matrix)). Any statistical approaches known in the art may be used to determine the expression level.

For example, the expression level may reflect the median expression level, mean expression level, or a numerical value that reflects the aggregated Z-score expression level for the combination of genes assayed (e.g., for at least one of the genes described herein (e.g., genes in a M1 macrophage gene signature set or genes in a gene signature matrix)).

In some instances, the expression level reflects the median normalized expression level, mean normalized expression level, or a numerical value that reflects the aggregated Z-score normalized expression level for the combinations of genes assayed (e.g., for at least one of the genes described herein (e.g., genes in a M1 macrophage gene signature set or genes in a gene signature matrix)).

### D. Determination of Th2 Biomarkers

### (i) Detection Methods

The Th2 biomarkers described herein may be based on an amount or expression level of a nucleic acid (e.g., an mRNA), a protein, or a cell (e.g., a T cells, e.g., a Th2 cell). Presence and/or expression levels/amount of the genes described herein (see, e.g., Table 5) can be determined qualitatively and/or quantitatively based on any suitable criterion known in the art, including but not limited to DNA, mRNA, cDNA, proteins, protein fragments, and/or gene copy number. Presence and/or levels/amounts of the cells described herein can be determined qualitatively and/or quantitatively based on any suitable criterion known in the art, including but not limited to microscopy, cytometry, DNA, mRNA, cDNA, proteins, protein fragments, and/or gene copy number.

TABLE 5
Exemplary Th2 gene signature sets

| Gene | |
|---|---|
| signature | |
| set | Genes |
| 1 | GZMK, IL5, IL13, MAD2L1, RRM2, BAG2, CXCR6, and CEP55 |
| 2 | IL5, IL13, MAD2L1, BAG2, CXCR6, RRAS2, CEP55, NUP37, and NPHP4 |
| 3 | GPR15, GZMA, IL5, IL13, SMAD2, CDK2AP1, RGS9, BAG2, SLC25A44, RAD50, CXCR6, |
| | TMEM39B, UBAP2, THADA, RNF34, and NPHP4 |
| 4 | ADCY1, AHI1, ANK1, BIRC5, CDC25C, CDC7, CENPF, CXCR6, DHFR, EVI5, GATA3, |
| | GSTA4, HELLS, IL26, LAIR2, LIMA1, MB, MICAL2, NEIL3, PHEX, PMCH, PTGIS, |
| | SLC39A14, SMAD2, SNRPD1, and WDHD1 |
| 5 | ASB2, CSRP2, DAPK1, DLC1, DNAJC12, DUSP6, GNAI1, LAMP3, NRP2, OSBPL1A, |
| | PDE4B, PHLDA1, PLA2G4A, RAB27B, RBMS3, RNF125, TMPRSS3, and GATA3 |

In some instances, nucleic acid expression levels of the genes described herein may be measured by polymerase chain reaction (PCR)-based assays, e.g., quantitative PCR, real-time PCR, quantitative real-time PCR (qRT-PCR), reverse transcriptase PCR (RT-PCR), and reverse transcriptase quantitative PCR (RT-qPCR). Platforms for performing quantitative PCR assays include Fluidigm (e.g., BIOMARKTM HD System). Other amplification-based methods include, for example, transcript-mediated amplification (TMA), strand displacement amplification (SDA), nucleic acid sequence based amplification (NASBA), and signal amplification methods such as bDNA.

In some instances, nucleic acid expression levels of the genes described herein also may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene.

Primers and probes may be labeled with a detectable marker, such as, for example, a radioisotope, fluorescent compound, bioluminescent compound, a chemiluminescent compound, metal chelator, or enzyme. Such probes and primers can be used to detect the presence of expressed genes (e.g., the genes described herein) in a sample. As will be understood by the skilled artisan, many different primers and probes may be prepared based on the sequences provided herein (or, in the case of genomic DNA, their adjacent sequences) and used effectively to amplify, clone, and/or determine the presence and/or expression levels of the genes described herein.

Other methods to detect nucleic acid expression levels of the genes described herein include electrophoresis, Northern and Southern blot analyses, in situ hybridization (e.g., single or multiplex nucleic acid in situ hybridization), RNAse protection assays, and microarrays (e.g., Illumina BEADARRAYTM technology; Beads Array for Detection of Gene Expression (BADGE)).

In some instances, the Th2 biomarker can be analyzed by a number of methodologies, including, but not limited to, RNA-seq, PCR, RT-qPCR, qPCR, multiplex qPCR, multiplex RT-qPCR, NANOSTRING® nCOUNTER® Gene Expression Assay, microarray analysis, serial analysis of gene expression (SAGE), Northern blot analysis, MassARRAY, ISH, whole genome sequencing, FACS, spatial transcriptomics, spatial proteomics, Western blot, ELISA, immunoprecipitation, immunohistochemistry, immunofluorescence, radioimmunoassay, dot blotting, immunodetection methods, surface plasmon resonance, optical spectroscopy, mass spectrometery, and HPLC, or combinations thereof.

### (ii) RT-qPCR

In some instances, nucleic acid expression levels of the genes described herein (e.g., genes in a Th2 cell gene signature set or genes in a gene signature matrix) can be detected using reverse transcription quantitative polymerase chain reaction (RT-qPCR). The technique of RT-qPCR is a form of PCR wherein the nucleic acid to be amplified is RNA that is first reverse transcribed into cDNA and the amount of PCR product is measured at each step in a PCR reaction. As RNA cannot serve as a template for PCR, the first step in gene expression profiling by PCR is the reverse transcription of the RNA template into cDNA, followed by its amplification in a PCR reaction. For example, reverse transcriptases may include avilo myeloblastosis virus reverse transcriptase (AMY-RD or Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

A variation of the PCR technique is quantitative real time PCR (qRT-PCR), which measures PCR product accumulation through a dual-labeled fluorigenic probe (i.e., TAQMAN® probe). The technique of quantitative real time polymerase chain reaction refers to a form of PCR wherein the amount of PCR product is measured at each step in a PCR reaction. This technique has been described in various publications including Cronin et al., Am. J. Pathol. 164(I): 35-42 (2004); and Ma et al., Cancer Cell 5: 607-616 (2004). Real time PCR is compatible both with quantitative competitive PCR, where an internal competitor for each target sequence is used for normalization, and/or with quantitative comparative PCR using a normalization gene contained within the sample, ora housekeeping gene for PCR. For further details see, e.g., Held et al., Genome Research 6: 986-994 (1996).

The steps of a representative protocol for profiling gene expression using fixed, paraffin- embedded tissues as the RNA source, including mRNA isolation, purification, primer extension and amplification are given in various published journal articles (for example: Godfrey et al., Malec. Diagnostics 2: 84-91 (2000); Specht et al., Am. J. Pathol. 158: 419-29 (2001)). Briefly, a representative process starts with cutting a section (e.g., a 10 microgram section) of a paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

The nucleic acid expression level determined by an amplification-based method (e.g., RT-qPCR) may be expressed as a cycle threshold value (Ct). From this value, a normalized expression level for each gene can be determined, e.g., using the delta Ct (dCt) method as follows: Ct(Control/Reference Gene) - Ct (Gene of Interest/Target Gene) =dCt (Gene of Interest/Target Gene). One of skill in the art will appreciate that the dCt value obtained may be a negative dCt value or a positive dCt value. As defined herein, a higher dCt value indicates a higher expression level of the gene of interest relative to the control gene. Conversely, a lower dCt value indicates a lower expression level of the gene of interest relative to the control gene. In cases where the expression levels of a plurality of genes has been determined, the expression level for each gene, e.g., expressed as a dCt value, may then be used to determine a single value that represents an aggregate or composite expression level for the plurality of genes (e.g., genes in a Th2 cell gene signature set). The aggregate or composite expression level may be the mean or median of dCt values determined for each target gene/gene of interest. As defined herein, a higher averaged dCt or median dCt value indicates a higher aggregative expression level of the plurality of target genes relative to the control gene (or plurality of control genes). A lower averaged dCt or median dCt value indicates a lower aggregative expression level of the plurality of target genes relative to the control gene (or plurality of control genes). Expression levels may be compared to a reference level.

In one particular instance, the nucleic acid expression levels described herein may be determined using a method including:

(a) obtaining or providing a sample from the individual, wherein the sample includes a tumor tissue sample (e.g., a paraffin-embedded, formalin-fixed tumor tissue sample);

(b) isolating mRNA from said sample;

(c) performing reverse transcription of the mRNA into cDNA (e.g., for at least one of the genes described herein (e.g., genes in a Th2 cell gene signature set or genes in a gene signature matrix));

(d) amplifying the cDNA (e.g., for at least one of the genes described herein (e.g., genes in a Th2 cell gene signature set or genes in a gene signature matrix)) using PCR; and

(e) quantifying the nucleic acid expression levels (e.g., for at least one of the genes described herein (e.g., genes in a Th2 cell gene signature set or genes in a gene signature matrix)).

One or more genes (e.g., one, two, three, four, five, six, seven, eight, nine, ten or more genes (e.g., 55, 82, 89, 106, 153, or 170 genes)) may be detected in a single assay depending on the primers or probes used. Further, the assay may be performed across one or more tubes (e.g., one, two, three, four, five, six, seven, eight, nine, ten or more tubes (e.g., 55, 82, 89, 106, 153, or 170 tubes)).

In some instances, the method further comprises (f) normalizing the nucleic acid expression level of the gene(s) (e.g., at least one of the genes described herein (e.g., genes in a Th2 cell gene signature set or genes in a gene signature matrix)) in said sample to the expression level of one or more reference genes (e.g., one, two, three, four, five, six, seven, eight, nine, or more reference genes, e.g., a housekeeping gene (e.g., β-actin)). For example, RT-qPCR may be used to analyze the expression level of the genes described herein ((e.g., at least one of the genes described herein (e.g., genes in a Th2 cell gene signature set or genes in a gene signature matrix)) to generate an expression level that reflects a normalized, averaged dCT value for the analyzed genes.

### (iii) RNA-Seq and Microarray

In some instances, nucleic acid expression levels of the genes described herein (e.g., genes in a Th2 cell gene signature set or genes in a gene signature matrix) can be detected using RNA-seq. RNA-seq, also called Whole Transcriptome Shotgun Sequencing (WTSS), refers to the use of high-throughput sequencing technologies to sequence and/or quantify cDNA in order to obtain information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009). In some instances, sequencing quality control is performed. In some instances, counts are normalized to transcripts per million (TPM).

### (c) Marker Gene Approaches

Marker gene approaches use the expression of one or more genes within a gene signature set to determine a Th2 biomarker (e.g., a number of Th2 cells in a sample). In some instances, the marker gene approach uses xCell (see, e.g., Aran et al. Genome Biol. 18(1): 220 (2017)).

The gene signature sets exemplified in Table 5 may be modified to remove, substitute, or add genes. In some instances, the number of genes in any of the exemplified gene signature sets can be increased or reduced by one or more genes (e.g., one, two, three, four, five, six, seven, eight, nine, ten, or more genes). In some instances, the number of genes in any of the exemplified gene signature sets can be increased or reduced by between about 5% and about 20% (e.g., 5%, 6%, 7%, 8%, 9%, 10%, 11%, 12%, 13%, 14%, 15%, 16%, 17%, 18%, 19%, or 20%). In some instances, genes from any of the exemplified gene signature sets can be substituted with genes in the same signaling pathway. In some instances, genes from any of the exemplified gene signature sets can be added to a different exemplified gene signature set (e.g., a gene from gene signature set 1 (e.g., GZMK) can be added to gene signature set 2) to generate a gene signature set. In some instances, a gene signature set can comprise genes that are present in most exemplified gene signature sets (e.g., IL5, IL13, BAG2, and CXCR6). In some instances, a gene signature set comprises IL5 and IL13. In some instances, a gene signature set comprises BAG2 and IL13. In some instances, a gene signature set comprises BAG2 and CXCR6. In some instances, a gene signature set comprises IL5, IL13, and BAG2. In some instances, a gene signature set comprises IL5, IL13, and CXCR6. In some instances, a gene signature set comprises IL13, BAG2, and CXCR6. In some instances, a gene signature set comprises IL13, BAG2, and CXCR6. In some instances, a gene signature set comprises IL5, IL13, BAG2, and CXCR6. In some instances, a gene signature set comprises IL5, IL13, MAD2L1, BAG2, CXCR6, and CEP55. In some instances, a gene signature set comprises IL5, IL13, MAD2L1, RRM2, BAG2, CXCR6, and CEP55. In some instances, a gene signature set comprises GZMK, IL5, IL13, MAD2L1, BAG2, CXCR6, and CEP55.

The steps of a representative protocol for determining a number of Th2 cells using an RNA-seq marker gene approach can be found in Aran et al. Genome Biol. 18(1): 220 (2017). Briefly, sequence scores (e.g., pre-processed raw sequence reads) obtained from a sample are transformed based on a power function derived from synthetic mixtures of a cell type (e.g., Th2 cells) and a control cell type (e.g., multipotent progenitor cells or endothelial cells) within a range (e.g., 0.8% to 25.6%) based on expected abundance of the cell type present in the sample. The transformed scores are further adjusted using a spillover compensation matrix (limited to 0.5 off the diagonal) derived from synthetic mixtures of 25% of a cell type (e.g., Th2 cells) and 75% of a control cell type (e.g., multipotent progenitor cells or endothelial cells). The final adjusted score represents the fraction of the cell type present in the sample.

### (d) Deconvolution Approaches

Deconvolution approaches use the expression of one or more genes within a gene signature matrix to determine a Th2 biomarker (e.g., a number of Th2 cells in a sample). In some instances, the deconvolution approach uses quanTlseq (see, e.g., Finotello et al. Genome Med. 11(1): 34 (2019)).

The steps of a representative protocol for determining a number of Th2 cells using an RNA-seq deconvolution approach can be found in Finotello et al. Genome Med. 11(1): 34 (2019). Briefly, sequence scores (e.g., pre-processed raw sequence reads) obtained from a sample are normalized and deconvoluted using a gene signature matrix to calculate proportions of cell types (e.g., Th2 cells, macrophages, M2 macrophages, B cells, monocytes, neutrophils, NK cells, non-regulatory CD4⁺ T cells, CD8⁺ T cells, regulatory T cells, dendritic cells, or other cell types) present in the sample using constrained least squares regression.

### (iv) Immunohistochemistry

In some instances, T cells (e.g., Th2 cells) can be detected using immunohistochemistry (IHC). In some instances, any of the genes described herein (e.g., genes in a Th2 cell gene signature set, or genes in a gene signature matrix) may be used to identify T cells (e.g., Th2 cells) or distinguish T cells from other cell types. In some instances, an antibody specific for any of the genes described herein is used as a primary antibody in the IHC assay. In some instances, a horseradish peroxidase (HRP)-conjugated secondary antibody is used in the IHC assay. In some instances, a signal from the IHC assay is compared to an IHC assay performed with a negative control antibody.

### (v) Flow Cytometry

In some instances, T cells (e.g., Th2 cells) can be detected using flow cytometry. In some instances, any of the genes described herein (e.g., genes in a Th2 cell gene signature set or genes in a gene signature matrix) may be used to identify T cells (e.g., Th2 cells) or distinguish T cells from other cell types. In some instances, an antibody specific for any of the genes described herein is used to label T cells (e.g., Th2 cells).

### (vi) Samples

The sample may be taken from an individual who is suspected of having, or is diagnosed as having, a lymphoma, and hence is likely in need of treatment, or from a healthy individual who is not suspected of having a lymphoma or who does not have lymphoma but has a family history of a lymphoma. For assessment of gene expression, samples, such as those containing cells, or proteins or nucleic acids produced by these cells, may be used in the methods of the present invention. The expression level of a gene can be determined by assessing the amount (e.g., the absolute amount or concentration) of the markers in a sample (e.g., a tissue sample, e.g., a tumor tissue sample, such as a biopsy). In addition, the level of a gene can be assessed in bodily fluids or excretions containing detectable levels of genes. Bodily fluids or secretions useful as samples in the present invention include, e.g., blood, urine, saliva, stool, pleural fluid, lymphatic fluid, sputum, ascites, prostatic fluid, cerebrospinal fluid (CSF), or any other bodily secretion or derivative thereof. The word blood is meant to include whole blood, plasma, serum, or any derivative of blood. Assessment of a gene in such bodily fluids or excretions can sometimes be preferred in circumstances where an invasive sampling method is inappropriate or inconvenient. In other embodiments, a tumor tissue sample is preferred.

The sample may be frozen, fresh, fixed (e.g., formalin fixed), centrifuged, and/or embedded (e.g., paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation, such as formalin fixation.

In one particular instance, the sample is a clinical sample. In another instance, the sample is used in a diagnostic assay, such as a diagnostic assay or diagnostic method of the invention. In some instances, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For example, samples of lymphoma lesions may be obtained by resection, fine needle aspiration, pleural fluid, or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In some instances, the sample from the individual is a tissue sample, a whole blood sample, a plasma sample, a serum sample, or a combination thereof. In some instances, the sample is a tissue sample. In some instances, the sample is a tumor tissue sample. In some instances, the sample is obtained prior to treatment. In some instances, the tissue sample is formalin-fixed and paraffin-embedded (FFPE) sample, an archival sample, a fresh sample, or a frozen sample. In some instances, the sample from the individual is a tissue sample. In some instances, the tissue sample is a tumor tissue sample (e.g., biopsy tissue). In some instances, the tumor tissue sample includes tumor cells, tumor infiltrating immune cells, stromal cells, normal adjacent tissue (NAT) cells, or a combination thereof. In some instances, the tissue sample is a biopsy. In some instances, the tissue sample is blood cells, lymph nodes, or bone/bone marrow.

In some instances, the tumor tissue sample is extracted from a malignant cancerous tumor (i.e., cancer). In some instances, the cancer is a solid tumor, or a non-solid or soft tissue tumor. In some instances, the tumor tissue sample is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) sample.

### (vii) RNA Extraction

Prior to detecting the level of a nucleic acid, mRNA may be isolated from a target sample. In some instances, the mRNA is total RNA isolated from tumors or tumor cell lines or, alternatively, normal tissues or cell lines. If the source of mRNA is a primary tumor, mRNA can be extracted, for example, from frozen or archived paraffin-embedded and fixed (e.g., formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56: A67 (1987), and De Andres et al., Bio Techniques 18: 42044 (1995). In particular, RNA isolation can be performed using a purification kit, buffer set, and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated, for example, by using RNA Stat-60 (TelTest). RNA prepared from tumor tissue samples can also be isolated, for example, by cesium chloride density gradient centrifugation.

### (viii) Expression Level

The expression level may reflect the expression levels of one or more genes described herein (e.g., one or more genes in a Th2 cell gene signature set or one or more genes in a gene signature matrix). In certain instances, the detected expression level of each gene is normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for both differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping gene (e.g., β-actin)). For example, in some instances, the nucleic acid expression levels detected using the methods described herein (e.g., for at least one of the genes described herein (e.g., genes in a Th2 cell gene signature set or genes in a gene signature matrix)) may be normalized to the expression level of one or more reference genes (e.g., one, two, three, four, five, six, seven, eight, nine, or more reference genes, e.g., a housekeeping gene (e.g., β-actin)). Alternatively, normalization can be based on the average signal or median signal of all of the assayed genes. On a gene-by-gene basis, a measured normalized amount of an mRNA can be compared to the amount found in a reference expression level. The presence and/or expression level/amount measured in a particular subject sample to be analyzed will fall at some percentile within this range, which can be determined by methods well known in the art.

In other instances, to determine an expression level, the detected expression level of each assayed gene is not normalized.

The expression level may reflect the aggregate or composite expression level of a single gene or a plurality of genes described herein (e.g., for at least one of the genes described herein (e.g., genes in a Th2 cell gene signature set or genes in a gene signature matrix)). Any statistical approaches known in the art may be used to determine the expression level.

For example, the expression level may reflect the median expression level, mean expression level, or a numerical value that reflects the aggregated Z-score expression level for the combination of genes assayed (e.g., for at least one of the genes described herein (e.g., genes in a Th2 cell gene signature set or genes in a gene signature matrix)).

In some instances, the expression level reflects the median normalized expression level, mean normalized expression level, or a numerical value that reflects the aggregated Z-score normalized expression level for the combinations of genes assayed (e.g., for at least one of the genes described herein (e.g., genes in a Th2 cell gene signature set or genes in a gene signature matrix)).

### IV. Therapeutic Methods, Compositions, and Uses

Provided herein are methods, compositions, and uses thereof, for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the method including administering to the patient an effective amount of an anti-CD20 antibody (e.g., obinutuzumab or rituximab) based on a macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) or a Th2 biomarker (e.g., an amount of Th2 cells) that has been determined in a sample (e.g., a tissue sample, e.g., a tumor tissue sample, such as a biopsy) from the patient.

In some instances, the anti-CD20 antibody may be administered as a first-line therapy. Alternatively, the anti-CD20 antibody may be administered as a second-line therapy.

In any of the below sections, the lymphoma can be a B-cell lymphoma. In some instances, the B-cell lymphoma is a non-Hodgkin lymphoma. In some instances, the non-Hodgkin lymphoma is a DLBCL. In some instances, the DLBCL is a germinal-center B-cell-like or activated B-cell-like diffuse large B-cell lymphoma. In some instances, the non-Hodgkin lymphoma is a marginal zone lymphoma. In some instances, the marginal zone lymphoma is an extranodal, nodal, or splenic marginal zone lymphoma. In some instances, the lymphoma is an indolent lymphoma. In some instances, the lymphoma is a follicular lymphoma (FL). In some instances, the lymphoma is a chronic lymphocytic leukemia (CLL). In some instances, the lymphoma is a CD20-positive lymphoma.

In any of the below sections, the patient is a human. In some instances, the patient has had no prior treatment. In some instances, the patient has had prior treatment. In some instances, the patient was previously treated with an anti-CD20 antibody. In some instances, the patient has not been previously treated with an anti-CD20 antibody.

In any of the below sections, the sample can be a tissue sample, a tumor sample, a whole blood sample, a plasma sample, or a serum sample. In some instances, the tissue sample is a tumor tissue sample. In some instances, the tumor tissue sample contains tumor cells, tumor-infiltrating immune cells, stromal cells, normal adjacent tissue (NAT) cells, or a combination thereof. In some instances, the tumor tissue sample is a biopsy. In some instances, the sample is an archival sample, a fresh sample, or a frozen sample.

In any of the below sections, the macrophage biomarker can be directly or indirectly measured. In some instances, the macrophage biomarker is a cell, nucleic acid, protein, lipid, or carbohydrate. In some instances, the macrophage biomarker is a gene expression value. In some instances, the macrophage biomarker is an amount of macrophages. In some instances, the amount of macrophages is an amount of M1 macrophages.

In any of the below sections, the Th2 biomarker can be directly or indirectly measured. In some instances, the Th2 biomarker is a cell, nucleic acid, protein, lipid, or carbohydrate. In some instances, the Th2 biomarker is a gene expression value. In some instances, the Th2 biomarker is an amount of T cells (e.g., an amount of Th2 cells). In some instances, the amount of T cells is an amount of Th2 cells.

### A. Macrophage Biomarkers for Use in Therapeutic Methods

In particular instances, the methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an effective amount of an anti-CD20 antibody (e.g., obinutuzumab or rituximab) are based on a macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) that has been determined in a sample (e.g., a tissue sample, e.g., a tumor tissue sample, such as a biopsy) from the patient.

In one aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the method including: (a) measuring a macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) in a sample (e.g., a tissue sample, e.g., a tumor tissue sample, such as a biopsy) from the patient, wherein the amount or level of the macrophage biomarker in the sample is above a reference macrophage biomarker amount or level, and (b) administering an effective amount of an anti-CD20 antibody to the patient based on the macrophage biomarker measured in step (a).

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the method including administering to the patient an effective amount of an anti-CD20 antibody, wherein prior to treatment the amount or level of a macrophage biomarker macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) in a sample (e.g., a tissue sample, e.g., a tumor tissue sample, such as a biopsy) from the patient has been determined to be above a reference macrophage biomarker amount or level.

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker macrophage biomarker (e.g., a gene expression value (e.g., a gene expression value derived from any of the gene signature sets described herein (e.g., any of the exemplified gene signature sets in Table 1 and Table 2)) or an amount of macrophages (e.g., M1 macrophages)) in a sample (e.g., a tissue sample, e.g., a tumor tissue sample, such as a biopsy) from the patient that is above a reference macrophage biomarker amount or level including administering to the patient an effective amount of an anti-CD20 antibody.

The amount or level of the macrophage biomarker that determines the various methods described herein are further described below.

### (i) Increased Macrophage Biomarker

An amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is above a reference macrophage biomarker amount or level may determine that a patient is to be administered an anti-CD20 antibody (e.g., obinutuzumab or rituximab).

For example, in some instances, a method of treating a patient with an amount or level of a macrophage biomarker in a sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the macrophage biomarker in the reference population comprises administering an anti-CD20 antibody (e.g., obinutuzumab or rituximab).

In some instances, a method of treating a patient with an amount or level of a macrophage biomarker in a sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55th percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the macrophage biomarker in the reference population comprises administering an anti-CD20 antibody (e.g., obinutuzumab or rituximab). For example, in some instances, a method of treating a patient with an amount or level of a macrophage biomarker in a sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population comprises administering an anti-CD20 antibody (e.g., obinutuzumab or rituximab).

In some instances, a method of treating a patient with an amount or level of a macrophage biomarker in a sample that is in about the top 80th percentile (i.e., equal to, or higher than, the 20% prevalence level) of the reference population comprises administering an anti-CD20 antibody (e.g., obinutuzumab or rituximab). In some instances, a method of treating a patient with an amount or level of a macrophage biomarker in a sample that is in about the top 75th percentile (i.e., equal to, or higher than, the 25% prevalence level) of the reference population comprises administering an anti-CD20 antibody (e.g., obinutuzumab or rituximab). In some instances, a method of treating a patient with an amount or level of a macrophage biomarker in a sample that is in about the top 50th percentile (i.e., equal to, or higher than, the 50% prevalence level) of the reference population comprises administering an anti-CD20 antibody (e.g., obinutuzumab or rituximab). In some instances, a method of treating a patient with an amount or level of a macrophage biomarker in a sample that is in about the top 25th percentile (i.e., equal to, or higher than, the 75% prevalence level) of the reference population comprises administering an anti-CD20 antibody (e.g., obinutuzumab or rituximab). In some instances, a method of treating a patient with an amount or level of a macrophage biomarker in a sample that is in about the top 20th percentile (i.e., equal to, or higher than, the 80% prevalence level) of the reference population comprises administering an anti-CD20 antibody (e.g., obinutuzumab or rituximab).

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Decreased Macrophage Biomarker

An amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is below a reference macrophage biomarker amount or level may determine that a patient is not to be administered an anti-CD20 antibody (e.g., obinutuzumab or rituximab).

In some instances, a method of treating a patient with an amount or level of a macrophage biomarker in a sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population comprises administering a therapy that does not include an anti-CD20 antibody (e.g., obinutuzumab or rituximab).

In some instances, a method of treating a patient with an amount or level of a macrophage biomarker in a sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population comprises administering a therapy that does not include an anti-CD20 antibody (e.g., obinutuzumab or rituximab). For example, in some instances, a method of treating a patient with an amount or level of a macrophage biomarker in a sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population comprises administering a therapy that does not include an anti-CD20 antibody (e.g., obinutuzumab or rituximab).

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (iii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%.

In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes two or more (e.g., two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iv) Indications

The methods described herein are useful for treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) with an anti-CD20 antibody (e.g., obinutuzumab or rituximab), the method including determining that a macrophage biomarker in a sample from the patient is an amount or level that is above a reference macrophage biomarker amount or level. Alternatively, an amount or level of the macrophage biomarker in the sample that is determined to be below a reference macrophage biomarker amount indicates that the patient is to be administered a therapy that does not include an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (v) Treatment Benefits

A patient who benefits from receiving treatment with an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in OS, PFS, CR, PR, or a combination thereof.

In some instances, a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample that is above a reference macrophage biomarker amount or level (e.g., an amount or level of a macrophage biomarker in a reference population) comprises administering an anti-CD20 antibody (e.g., obinutuzumab or rituximab) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample that is above a reference macrophage biomarker amount or level (e.g., an amount or level of a macrophage biomarker in a reference population) comprises administering an anti-CD20 antibody (e.g., obinutuzumab or rituximab) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### B. Th2 Biomarkers for Use in Therapeutic Methods

In particular instances, the methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an effective amount of an anti-CD20 antibody (e.g., obinutuzumab or rituximab) are based on a Th2 biomarker (e.g., a gene expression value) or an amount of T cells (e.g., Th2 cells)) that has been determined in a sample (e.g., a tissue sample, e.g., a tumor tissue sample, such as a biopsy) from the patient.

In one aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the method including: (a) measuring a Th2 biomarker (e.g., a gene expression value) or an amount of T cells (e.g., Th2 cells)) in a sample (e.g., a tissue sample, e.g., a tumor tissue sample, such as a biopsy) from the patient, wherein the amount or level of the Th2 biomarker in the sample is above a reference Th2 biomarker amount or level, and (b) administering an effective amount of an anti-CD20 antibody to the patient based on the Th2 biomarker measured in step (a).

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the method including administering to the patient an effective amount of an anti-CD20 antibody, wherein prior to treatment the amount or level of a Th2 biomarker Th2 biomarker (e.g., a gene expression value) or an amount of T cells (e.g., Th2 cells)) in a sample (e.g., a tissue sample, e.g., a tumor tissue sample, such as a biopsy) from the patient has been determined to be above a reference Th2 biomarker amount or level.

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker Th2 biomarker (e.g., a gene expression value) or an amount of T cells (e.g., Th2 cells)) in a sample (e.g., a tissue sample, e.g., a tumor tissue sample, such as a biopsy) from the patient that is above a reference Th2 biomarker amount or level including administering to the patient an effective amount of an anti-CD20 antibody. The amount or level of the Th2 biomarker that determines the various methods described herein are further described below.

### (i) Increased Th2 Biomarker

An amount or level of the Th2 biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) that is above a reference Th2 biomarker amount or level may determine that a patient is to be administered an anti-CD20 antibody (e.g., obinutuzumab or rituximab).

For example, in some instances, a method of treating a patient with an amount or level of a Th2 biomarker in a sample that is in about the top 99^{th} percentile (equal to, or higher than, about the 1% prevalence level), about the top 95^{th} (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the Th2 biomarker in the reference population comprises administering an anti-CD20 antibody (e.g., obinutuzumab or rituximab).

In some instances, a method of treating a patient with an amount or level of a Th2 biomarker in a sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the Th2 biomarker in the reference population comprises administering an anti-CD20 antibody (e.g., obinutuzumab or rituximab). For example, in some instances, a method of treating a patient with an amount or level of a Th2 biomarker in a sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population comprises administering an anti-CD20 antibody (e.g., obinutuzumab or rituximab).

In some instances, a method of treating a patient with an amount or level of a Th2 biomarker in a sample that is in about the top 80th percentile (i.e., equal to, or higher than, the 20% prevalence level) of the reference population comprises administering an anti-CD20 antibody (e.g., obinutuzumab or rituximab). In some instances, a method of treating a patient with an amount or level of a Th2 biomarker in a sample that is in about the top 75th percentile (i.e., equal to, or higher than, the 25% prevalence level) of the reference population comprises administering an anti-CD20 antibody (e.g., obinutuzumab or rituximab). In some instances, a method of treating a patient with an amount or level of a Th2 biomarker in a sample that is in about the top 50th percentile (i.e., equal to, or higher than, the 50% prevalence level) of the reference population comprises administering an anti-CD20 antibody (e.g., obinutuzumab or rituximab). In some instances, a method of treating a patient with an amount or level of a Th2 biomarker in a sample that is in about the top 25th percentile (i.e., equal to, or higher than, the 75% prevalence level) of the reference population comprises administering an anti-CD20 antibody (e.g., obinutuzumab or rituximab). In some instances, a method of treating a patient with an amount or level of a Th2 biomarker in a sample that is in about the top 20th percentile (i.e., equal to, or higher than, the 80% prevalence level) of the reference population comprises administering an anti-CD20 antibody (e.g., obinutuzumab or rituximab).

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to the amount or level of the Th2 biomarker in a reference sample, reference cell, reference tissue, control sample, control cell, or control tissue. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the Th2 biomarker in a reference sample, reference cell, reference tissue, control sample, control cell, or control tissue. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the Th2 biomarker in a reference sample, reference cell, reference tissue, control sample, control cell, or control tissue.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to a pre-assigned amount or level of the Th2 biomarker. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the Th2 biomarker. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the Th2 biomarker.

### (ii) Reference Th2 Biomarker

The reference Th2 biomarker amount or level can be a pre-assigned Th2 biomarker amount or level. In some instances, the amount or level of the Th2 biomarker in a reference population is a median amount or level of the Th2 biomarker of the reference population. In some instances, the amount or level of the Th2 biomarker in a reference population is a mean amount or level of the Th2 biomarker of the reference population.

In some instances, the pre-assigned Th2 biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 0%.

The reference amount or level of the Th2 biomarker described herein may be based on the amount or level of the Th2 biomarker in a reference population. In some instances, the reference Th2 biomarker described herein is an amount or level of the Th2 biomarker in a reference population that includes two or more (e.g., two or more, three or more, four or more, or five or more) subsets of patients.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the Th2 biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the Th2 biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference Th2 biomarker may be an amount or level of the Th2 biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iii) Indications

The methods described herein are useful for treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) with an anti-CD20 antibody (e.g., obinutuzumab or rituximab), the method including determining that a Th2 biomarker in a sample from the patient is an amount or level that is above a reference Th2 biomarker amount or level. Alternatively, an amount or level of the Th2 biomarker in the sample that is determined to be below a reference Th2 biomarker amount indicates that the patient is to be administered a therapy that does not include an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (iv) Treatment Benefits

A patient who benefits from receiving treatment with an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in OS, PFS, CR, PR, or a combination thereof.

In some instances, a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample that is above a reference Th2 biomarker amount or level (e.g., an amount or level of a Th2 biomarker in a reference population) comprises administering an anti-CD20 antibody (e.g., obinutuzumab or rituximab) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample that is above a reference Th2 biomarker amount or level (e.g., an amount or level of a Th2 biomarker in a reference population) comprises administering an anti-CD20 antibody (e.g., obinutuzumab or rituximab) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### C. Uses of an Anti-CD20 Antibody in the Manufacture of Medicaments

In a further aspect, the invention provides for the use of an anti-CD20 antibody (e.g., obinutuzumab or rituximab) in the manufacture or preparation of a medicament for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma)) with an amount or level of a macrophage biomarker in a sample from the patient that is above a reference macrophage biomarker amount or level. In some instances, the medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is above a reference Th2 biomarker amount or level.

The amount or level of the macrophage biomarker or Th2 biomarker that determines the various uses described herein are further described below.

### (i) Increased Macrophage Biomarker

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the macrophage biomarker in the reference population.

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the macrophage biomarker in the reference population. For example, in some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 80th percentile (i.e., equal to, or higher than, the 20% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 75th percentile (i.e., equal to, or higher than, the 25% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 50th percentile (i.e., equal to, or higher than, the 50% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 25th percentile (i.e., equal to, or higher than, the 75% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample from the patient that is in about the top 20th percentile (i.e., equal to, or higher than, the 80% prevalence level) of the reference population.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes two or more (e.g., two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iii) Increased Th2 Biomarker

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the Th2 biomarker in the reference population.

In some instances, a medicament is for use in a method of treating a patient having a lymphoma 35 (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the Th2 biomarker in the reference population. For example, in some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 80th percentile (i.e., equal to, or higher than, the 20% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 75th percentile (i.e., equal to, or higher than, the 25% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 50th percentile (i.e., equal to, or higher than, the 50% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample from the patient that is in about the top 25th percentile (i.e., equal to, or higher than, the 75% prevalence level) of the reference population. In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma)) with an amount or level of a Th2 biomarker in a sample from the patient that is in about the top 20th percentile (i.e., equal to, or higher than, the 80% prevalence level) of the reference population.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to the amount or level of the Th2 biomarker in a reference sample, reference cell, reference tissue, control sample, control cell, or control tissue. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the Th2 biomarker in a reference sample, reference cell, reference tissue, control sample, control cell, or control tissue. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the Th2 biomarker in a reference sample, reference cell, reference tissue, control sample, control cell, or control tissue.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to a pre-assigned amount or level of the Th2 biomarker. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the Th2 biomarker. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the Th2 biomarker.

### (iv) Reference Th2 Biomarker

The reference Th2 biomarker amount or level can be a pre-assigned Th2 biomarker amount or level. In some instances, the amount or level of the Th2 biomarker in a reference population is a median amount or level of the Th2 biomarker of the reference population. In some instances, the amount or level of the Th2 biomarker in a reference population is a mean amount or level of the Th2 biomarker of the reference population.

In some instances, the pre-assigned Th2 biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 0%.

The reference amount or level of the Th2 biomarker described herein may be based on the amount or level of the Th2 biomarker in a reference population. In some instances, the reference Th2 biomarker described herein is an amount or level of the Th2 biomarker in a reference population that includes two or more (e.g., two or more, three or more, four or more, or five or more) subsets of patients. In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on a hazard ratio (HR) determined from an analysis of the amount or level of the Th2 biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the Th2 biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference Th2 biomarker may be an amount or level of the Th2 biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (v) Indications

The medicaments described herein are useful for treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) with an anti-CD20 antibody (e.g., obinutuzumab or rituximab), the method including determining that a macrophage biomarker in a sample from the patient is an amount or level that is above a reference macrophage biomarker amount or level. Alternatively, an amount or level of the macrophage biomarker in the sample that is determined to be below a reference macrophage biomarker amount indicates that the patient is to be administered a therapy that does not include an anti-CD20 antibody (e.g., obinutuzumab or rituximab). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma))) with an anti-CD20 antibody (e.g., obinutuzumab or rituximab), the method including determining that a Th2 biomarker in a sample from the patient is an amount or level that is above a reference Th2 biomarker amount or level. Alternatively, an amount or level of the Th2 biomarker in the sample that is determined to be below a reference Th2 biomarker amount indicates that the patient is to be administered a therapy that does not include an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (vi) Treatment Benefits

A patient who benefits from receiving treatment with a medicament may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the medicaments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in OS, PFS, CR, PR, or a combination thereof.

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample that is above a reference macrophage biomarker amount or level (e.g., an amount or level of a macrophage biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a macrophage biomarker in a sample that is above a reference macrophage biomarker amount or level (e.g., an amount or level of a macrophage biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample that is above a reference Th2 biomarker amount or level (e.g., an amount or level of a Th2 biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) with an amount or level of a Th2 biomarker in a sample that is above a reference Th2 biomarker amount or level (e.g., an amount or level of a Th2 biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### D. Therapeutic Uses of an Anti-CD20 Antibody

In a further aspect, the invention provides for the use of an anti-CD20 antibody (e.g., obinutuzumab or rituximab) in treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the anti-CD20 antibody (e.g., obinutuzumab or rituximab) is for use in the treatment of a patient having a lymphoma and having an amount or level of a macrophage biomarker in a sample from the patient that is above a reference macrophage biomarker amount or level.

In some instances, the anti-CD20 antibody (e.g., obinutuzumab or rituximab) is for use in the treatment of a patient having a lymphoma and having an amount or level of a Th2 biomarker in a sample from the patient that is above a reference Th2 biomarker amount or level.

The amount or level of the macrophage biomarker or Th2 biomarker that determines the various uses described herein are further described below.

### (i) Increased Macrophage Biomarker

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the macrophage biomarker in the reference population.

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the macrophage biomarker in the reference population. For example, In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 80th percentile (i.e., equal to, or higher than, the 20% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 75th percentile (i.e., equal to, or higher than, the 25% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 50th percentile (i.e., equal to, or higher than, the 50% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 25th percentile (i.e., equal to, or higher than, the 75% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample from the patient that is in about the top 20th percentile (i.e., equal to, or higher than, the 80% prevalence level) of the reference population.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is above a reference macrophage biomarker amount or level refers to an overall increase of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is above a reference macrophage biomarker amount or level refers to an increase in the amount or level of the macrophage biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is above a reference macrophage biomarker amount or level refers to an overall increase in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%.

In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes two or more (e.g., two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference macrophage biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on an HR determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on an HR determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (iii) Increased Th2 Biomarker

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40th percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the amount or level of the Th2 biomarker in the reference population.

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the amount or level of the Th2 biomarker in the reference population. For example, In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 80th percentile (i.e., equal to, or higher than, the 20% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 75th percentile (i.e., equal to, or higher than, the 25% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 50th percentile (i.e., equal to, or higher than, the 50% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 25th percentile (i.e., equal to, or higher than, the 75% prevalence level) of the reference population. In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample from the patient that is in about the top 20th percentile (i.e., equal to, or higher than, the 80% prevalence level) of the reference population.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to the amount or level of the Th2 biomarker in a reference sample, reference cell, reference tissue, control sample, control cell, or control tissue. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× the amount or level of the Th2 biomarker in a reference sample, reference cell, reference tissue, control sample, control cell, or control tissue. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the Th2 biomarker in a reference sample, reference cell, reference tissue, control sample, control cell, or control tissue.

In some instances, an amount or level of the Th2 biomarker that is above a reference Th2 biomarker amount or level refers to an overall increase of about 10%, 20%, 30%, 40%, 50%, 60%, 70%, 80%, 90%, 95%, 96%, 97%, 98%, or 99% or greater in the amount or level of the Th2 biomarker, detected by standard art-known methods such as those described herein, as compared to a pre-assigned amount or level of the Th2 biomarker. In certain instances, an amount or level of the Th2 biomarker that is above a reference Th2 biomarker amount or level refers to an increase in the amount or level of the Th2 biomarker in the sample, wherein the increase is at least about 1.5×, 1.75×, 2×, 3×, 4×, 5×, 6×, 7×, 8×, 9×, 10×, 25×, 50×, 75×, or 100× a pre-assigned amount or level of the Th2 biomarker. In some instances, an amount or level of the Th2 biomarker that is above a reference Th2 biomarker amount or level refers to an overall increase in the amount or level of the Th2 biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the Th2 biomarker.

### (iv) Reference Th2 Biomarker

The reference Th2 biomarker amount or level can be a pre-assigned Th2 biomarker amount or level. In some instances, the amount or level of the Th2 biomarker in a reference population is a median amount or level of the Th2 biomarker of the reference population. In some instances, the amount or level of the Th2 biomarker in a reference population is a mean amount or level of the Th2 biomarker of the reference population.

In some instances, the pre-assigned Th2 biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 0%.

The reference amount or level of the Th2 biomarker described herein may be based on the amount or level of the Th2 biomarker in a reference population. In some instances, the reference Th2 biomarker described herein is an amount or level of the Th2 biomarker in a reference population that includes two or more (e.g., two or more, three or more, four or more, or five or more) subsets of patients.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have been administered one or more doses (e.g., at least one, two, three, four, five, six, seven, eight, nine, or ten or more doses) of an anti-CD20 antibody (e.g., obinutuzumab or rituximab).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a monotherapy.

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference Th2 biomarker is an amount or level of the Th2 biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD20 antibody (e.g., obinutuzumab or rituximab) and a second subset of patients who have not been treated with an anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) above the reference Th2 biomarker, wherein the patient's responsiveness to treatment with the anti-CD20 antibody (e.g., obinutuzumab or rituximab) is significantly improved relative to the patient's responsiveness to treatment without the anti-CD20 antibody (e.g., obinutuzumab or rituximab).

In some instances, the reference amount or level of the Th2 biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the Th2 biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference Th2 biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab).

In some instances, an optimal separation or significant separation may be based on an HR determined from an analysis of the amount or level of the Th2 biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on an HR determined from an analysis of the 10 amount or level of the Th2 biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference Th2 biomarker may be an amount or level of the Th2 biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) or have a lymphoma but are treatment naïve.

### (v) Indications

The anti-CD20 antibodies (e.g., obinutuzumab and rituximab) described herein are useful for methods of treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) with an anti-CD20 antibody (e.g., obinutuzumab or rituximab), the method including determining that a macrophage biomarker in a sample from the patient is an amount or level that is above a reference macrophage biomarker amount or level. Alternatively, an amount or level of the macrophage biomarker in the sample that is determined to be below a reference macrophage biomarker amount indicates that the patient is to be administered a therapy that does not include an anti-CD20 antibody (e.g., obinutuzumab or rituximab). For example, the methods described herein may be used for treating a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma))) with an anti-CD20 antibody (e.g., obinutuzumab or rituximab), the method including determining that a Th2 biomarker in a sample from the patient is an amount or level that is above a reference Th2 biomarker amount or level. Alternatively, an amount or level of the Th2 biomarker in the sample that is determined to be below a reference Th2 biomarker amount indicates that the patient is to be administered a therapy that does not include an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### (vi) Treatment Benefits

A patient who benefits from receiving treatment with an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the anti-CD20 antibodies (e.g., obinutuzumab and rituximab) described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In some instances, the benefit may be an increase in OS, PFS, CR, PR, or a combination thereof.

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample that is above a reference macrophage biomarker amount or level (e.g., an amount or level of a macrophage biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a macrophage biomarker in a sample that is above a reference macrophage biomarker amount or level (e.g., an amount or level of a macrophage biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample that is above a reference Th2 biomarker amount or level (e.g., an amount or level of a Th2 biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

In some instances, the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) and having an amount or level of a Th2 biomarker in a sample that is above a reference Th2 biomarker amount or level (e.g., an amount or level of a Th2 biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD20 antibody (e.g., obinutuzumab or rituximab).

### E. Exemplary anti-CD20 Antibodies

### Type II Anti-CD20 Antibodies

In some instances, the above methods, compositions, and uses involve administration or use of type II anti-CD20 antibodies. In some instances, type II anti-CD20 antibodies mediate the death of cells that express CD20. In some instances, the type II anti-CD20 antibody is a monoclonal antibody. In some instances, the type II anti-CD20 antibody is an antibody fragment selected from the group consisting of Fab, Fab′-SH, Fv, scFv, and (Fab′)₂ fragments. In some instances, the type II anti-CD20 antibody is a humanized antibody. In some instances, the type II anti-CD20 antibody is a human antibody. In some instances, the type II anti-CD20 antibody described herein binds to human CD20. In some particular instances, the type II anti-CD20 antibody is obinutuzumab (see, e.g., WO 2005/044859 and U.S. Patent Publication No. 2005/0123546, which are incorporated by reference herein in entirety), which is incorporated by reference herein in its entirety). Obinutuzumab (Genentech) is also known as GAZYVATM/GAZYVAROTM and GA101.

In some instances, the type II anti-CD20 antibody comprises a heavy chain variable region (HVR-H) comprising an HVR-H1, HVR-H2, and HVR-H3 sequence, wherein:

(a) the HVR-H1 sequence is GYAFSY (SEQ ID NO: 1);

(b) the HVR-H2 sequence is FPGDGDTD (SEQ ID NO: 2); and

(c) the HVR-H3 sequence is NVFDGYWLVY (SEQ ID NO: 3).

In some instances, the type II anti-CD20 antibody comprises a heavy chain variable region (HVR-H) comprising an HVR-H1, HVR-H2, and HVR-H3 sequence, wherein:

(a) the HVR-H1 sequence is GYAFSY (SEQ ID NO: 27);

(b) the HVR-H2 sequence is FPGDGDTD (SEQ ID NO: 28); and

(c) the HVR-H3 sequence is NVFDGYWLVY (SEQ ID NO: 3).

In some instances, the type II anti-CD20 antibody further comprises a light chain variable region (HVR-L) comprising an HVR-L1, HVR-L2, and HVR-L3 sequence, wherein:

(a) the HVR-L1 sequence is RSSKSLLHSNGITYLY (SEQ ID NO: 4);

(b) the HVR-L2 sequence is QMSNLVS (SEQ ID NO: 5); and

(c) the HVR-L3 sequence is AQNLELPYT (SEQ ID NO: 6).

In some instances, the type II anti-CD20 antibody comprises a heavy chain and a light chain sequence, wherein:

(a) the heavy chain variable (VH) region
sequence comprises the amino acid sequence:
(SEQ ID NO: 7)
QVQLVQSGAEVKKPGSSVKVSCKASGYAFSYSWINWWRQAPGQGLEWMGR
IFPGDGDTDYNGKFKGRVTITADKSTSTAYMELSSLRSEDTAVYYCARNV
FDGYWLVYWGQGTLVTVSS;
and
(b) the light chain variable (VL) region
sequence comprises the amino acid sequence:
(SEQ ID NO: 8)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTV

In some instances, the type II anti-CD20 antibody comprises a heavy chain and a light chain sequence, wherein:

(a) the heavy chain comprises the amino
acid sequence:
(SEQ ID NO: 9)
QVQLVQSGAEVKKPGSSVKVSCKASGYAFSYSWINWWRQAPGQGLEWMGR
IFPGDGDTDYNGKFKGRVTITADKSTSTAYMELSSLRSEDTAVYYCARNV
FDGYWLVYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG;
and
(b) the light chain comprises the amino
acid sequence:
(SEQ ID NO: 10)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In some instances, the type II anti-CD20 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 7); (b) a VL domain comprising an amino acid sequence comprising having at least 95% sequence identity (e.g., at least 95%, 96%, 97%, 98%, or 99% sequence identity) to, or the sequence of (SEQ ID NO: 8); or (c) a VH domain as in (a) and a VL domain as in (b).

In another instances, a type II anti-CD20 antibody is provided, wherein the antibody comprises a VH as in any of the instances provided above, and a VL as in any of the instances provided above, wherein one or both of the variable domain sequences include post-translational modifications.

In certain instances, a type II anti-CD20 antibody may bind to CD20 on the surface of a lymphoma cell and induce apoptosis. In certain instances, a type II anti-CD20 antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M 10⁻¹³ M). In certain instances, a type II anti-CD20 antibody that binds to CD20 has a KD of <50 nM. In certain instances, a type II anti-CD20 antibody that binds to CD20 has a K_{D} of <20 nM. In certain instances, a type II anti-CD20 antibody that binds to CD20 has a K_{D} of <10 nM. In certain instances, the binding is at a K_{D} of <7.5 nM, <5 nM, between 1-5 nM, or <1 nM. In certain instances, the type II anti-CD20 antibody may bind to both human CD20 and cyno CD20.

In some instances, the methods or uses described herein may include using or administering an isolated anti-CD20 antibody that competes for binding to CD20 with any of the type II anti-CD20 antibodies described above. For example, the method may include administering an isolated anti-CD20 antibody that competes for binding to CD20 with a type II anti-CD20 antibody having the following six HVRs: (a) an HVR-H1 comprising the amino acid sequence of GYAFSY (SEQ ID NO: 1); (b) an HVR-H2 comprising the amino acid sequence of FPGDGDTD (SEQ ID NO: 2); (c) an HVR-H3 comprising the amino acid sequence of NVFDGYWLVY (SEQ ID NO: 3); (d) an HVR-L1 comprising the amino acid sequence of RSSKSLLHSNGITYLY (SEQ ID NO: 4), (e) an HVR-L2 comprising the amino acid sequence of QMSNLVS (SEQ ID NO: 5); and (f) an HVR-L3 comprising the amino acid sequence of AQNLELPYT (SEQ ID NO: 6). In another example, the method may include administering an isolated anti-CD20 antibody that competes for binding to CD20 with a type II anti-CD20 antibody having the following six HVRs: (a) an HVR-H1 comprising the amino acid sequence of YSWIN (SEQ ID NO: 27); (b) an HVR-H2 comprising the amino acid sequence of RIFPGDGDTDYNGKFK (SEQ ID NO: 28); (c) an HVR-H3 comprising the amino acid sequence of NVFDGYWLVY (SEQ ID NO: 3); (d) an HVR-L1 comprising the amino acid sequence of RSSKSLLHSNGITYLY (SEQ ID NO: 4), (e) an HVR-L2 comprising the amino acid sequence of QMSNLVS (SEQ ID NO: 5); and (f) an HVR-L3 comprising the amino acid sequence of AQNLELPYT (SEQ ID NO: 6). The methods described herein may also include administering an isolated anti-CD20 antibody that binds to the same epitope as a type II anti-CD20 antibody described above.

### Type I Anti-CD20 Antibodies

In some instances, the above methods, compositions, and uses involve administration or use of type I anti-CD20 antibodies. In some instances, type I anti-CD20 antibodies mediate the death of cells that express CD20. In some instances, the type I anti-CD20 antibody is a monoclonal antibody. In some instances, the type I anti-CD20 antibody is an antibody fragment selected from the group consisting of Fab, Fab′-SH, Fv, scFv, and (Fab′)₂ fragments. In some instances, the type I anti-CD20 antibody is a humanized antibody. In some instances, the type I anti-CD20 antibody is a human antibody. In some instances, the type I anti-CD20 antibody described herein binds to human CD20. In some particular instances, the type I anti-CD20 antibody is rituximab (RITUXAN®).

The terms “rituximab” or “RITUXAN®” herein refer to the genetically engineered chimeric murine/human monoclonal antibody directed against the CD20 antigen and designated “C2B8” in U.S. Pat. No. 5,736,137, which is incorporated herein by reference in its entirety. The antibody is an IgGI kappa immunoglobulin containing murine light and heavy chain variable region sequences and human constant region sequences. Rituximab has a binding affinity for the CD20 antigen of approximately 8.0 nM.

In certain instances, the type I anti-CD20 antibodies includes at least one, two, three, four, five, or six HVRs selected from: (a) an HVR-H1 comprising the amino acid sequence of SYNMH (SEQ ID NO: 11); (b) an HVR-H2 comprising the amino acid sequence of AIYPGNGDTSYNQKFKG (SEQ ID NO: 12); (c) an HVR-H3 comprising the amino acid sequence of STYYGGDVVYFNV (SEQ ID NO: 13); (d) an HVR-L1 comprising the amino acid sequence of RASSSVSYIH (SEQ ID NO: 14), (e) an HVR-L2 comprising the amino acid sequence of ATSNLAS (SEQ ID NO: 15); and/or (f) an HVR-L3 comprising the amino acid sequence of QQWTSNPPT (SEQ ID NO: 16), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 11-16.

In some instances, any of the above type I anti-CD20 antibodies includes (a) an HVR-H1 comprising the amino acid sequence of SYNMH (SEQ ID NO: 11); (b) an HVR-H2 comprising the amino acid sequence of AIYPGNGDTSYNQKFKG (SEQ ID NO: 12); (c) an HVR-H3 comprising the amino acid sequence of STYYGGDWYFNV (SEQ ID NO: 13); (d) an HVR-L1 comprising the amino acid sequence of RASSSVSYIH (SEQ ID NO: 14); (e) an HVR-L2 comprising the amino acid sequence of ATSNLAS (SEQ ID NO: 15); and (f) an HVR-L3 comprising the amino acid sequence of QQWTSNPPT (SEQ ID NO: 16).

In some instances, the type I anti-CD20 antibody further comprises at least one, two, three, or four of the following light chain variable region framework regions (FRs): an FR-L1 comprising the amino acid sequence of QIVLSQSPAILSASPGEKVTMTC (SEQ ID NO: 17); an FR-L2 comprising the amino acid sequence of WFQQKPGSSPKPWIY (SEQ ID NO: 18); an FR-L3 comprising the amino acid sequence of GVPVRFSGSGSGTSYSLTISRVEAEDAATYYC (SEQ ID NO: 19); and/or an FR-L4 comprising the amino acid sequence of FGGGTKLEIK (SEQ ID NO: 20), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 17-20. In some instances, for example, the antibody further comprises an FR-L1 comprising the amino acid sequence of QIVLSQSPAILSASPGEKVTMTC (SEQ ID NO: 17); an FR-L2 comprising the amino acid sequence of WFQQKPGSSPKPWIY (SEQ ID NO: 18); an FR-L3 comprising the amino acid sequence of GVPVRFSGSGSGTSYSLTISRVEAEDAATYYC (SEQ ID NO: 19); and an FR-L4 comprising the amino acid sequence of FGGGTKLEIK (SEQ ID NO: 20).

In some instances, the type I anti-CD20 antibody further comprises at least one, two, three, or four of the following heavy chain variable region FRs: an FR-H1 comprising the amino acid sequence of QVQLQQPGAELVKPGASVKMSCKASGYTFT (SEQ ID NO: 21); an FR-H2 comprising the amino acid sequence of VVVKQTPGRGLEWIG (SEQ ID NO: 22); an FR-H3 comprising the amino acid sequence of KATLTADKSSSTAYMQLSSLTSEDSAVYYCAR (SEQ ID NO: 23); and/or an FR-H4 comprising the amino acid sequence of WGAGTTVTVSA (SEQ ID NO: 24), ora combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 21-24. In some instances, the type I anti-CD20 antibody includes an FR-H1 comprising the amino acid sequence of QVQLQQPGAELVKPGASVKMSCKASGYTFT (SEQ ID NO: 21); an FR-H2 comprising the amino acid sequence of VVVKQTPGRGLEWIG (SEQ ID NO: 22); an FR-H3 comprising the amino acid sequence of KATLTADKSSSTAYMQLSSLTSEDSAVYYCAR (SEQ ID NO: 23); and an FR-H4 comprising the amino acid sequence of WGAGTTVTVSA (SEQ ID NO: 24).

In some instances, the type I anti-CD20 antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of QVQLQQPGAELVKPGASVKMSCKASGYTFTSYNMHWVKQTPGRGLEWIGAIYPGNGDTSYNQKFKGKATL TADKSSSTAYMQLSSLTSEDSAVYYCARSTYYGGDWYFNVWGAGTTVTVSA (SEQ ID NO: 25) and/or a VL domain comprising an amino acid sequence having at least 90% sequence identity (e.g., at least 91%, 92%, 93%, 94%, 95%, 96%, 97%, 98%, or 99% sequence identity) to, or the sequence of

(SEQ ID NO: 26)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIK

In another instances, a type I anti-CD20 antibody is provided, wherein the antibody comprises a VH as in any of the instances provided above, and a VL as in any of the instances provided above, wherein one or both of the variable domain sequences include post-translational modifications.

In certain instances, a type I anti-CD20 antibody may bind to CD20 on the surface of a lymphoma cell and mediate cell lysis through the activation of complement-dependent lysis, antibody-dependent cellular cytotoxicity (ADCC), and apoptosis mediated by Fc cross-linking. In certain instances, a type I anti-CD20 antibody that binds to CD20 has a dissociation constant (K_{D}) of ≤1 μM, ≤100 nM, ≤10 nM, 1 ≤nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain instances, a type I anti-CD20 antibody that binds to CD20 has a K_{D} of <10 nM. In certain instances, the binding is at a K_{D} of <7.5 nM, <5 nM, between 1-5 nM, or <1 nM. In certain instances, the type I anti-CD20 antibody may bind to both human CD20 and cyno CD20.

In some instances, the methods or uses described herein may include using or administering an isolated anti-CD20 antibody that competes for binding to CD20 with any of the type I anti-CD20 antibodies described above. For example, the method may include administering an isolated anti-CD20 antibody that competes for binding to CD20 with a type I anti-CD20 antibody having the following six HVRs: (a) an HVR-H1 comprising the amino acid sequence of SYNMH (SEQ ID NO: 11); (b) an HVR-H2 comprising the amino acid sequence of AIYPGNGDTSYNQKFKG (SEQ ID NO: 12); (c) an HVR-H3 comprising the amino acid sequence of STYYGGDVVYFNV (SEQ ID NO: 13); (d) an HVR-L1 comprising the amino acid sequence of RASSSVSYIH (SEQ ID NO: 14), (e) an HVR-L2 comprising the amino acid sequence of ATSNLAS (SEQ ID NO: 15); and (f) an HVR-L3 comprising the amino acid sequence of QQWTSNPPT (SEQ ID NO: 16). The methods described herein may also include administering an isolated anti-CD20 antibody that binds to the same epitope as a type I anti-CD20 antibody described above. In a further instance, an anti-CD20 antibody according to any of the above instances (e.g., a type II or a type I anti-CD20 antibody) may incorporate any of the features, singly or in combination, as described in Sections (i)-(v) below.

### (i) Substitution, Insertion, and Deletion Variants

In certain instances, the anti-CD20 antibody (e.g., obinutuzumab or rituximab) variants having one or more amino acid substitutions are provided for use in the methods, compositions, and/or kits of the invention. Sites of interest for substitutional mutagenesis include the HVRs and FRs. Conservative substitutions are shown in Table 6 under the heading of “preferred substitutions.” More substantial changes are provided in Table 6 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 6
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., ed., Human Press, Totowa, NJ, (2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs 25 so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### (ii) Glycosylation Variants

In some instances, the anti-CD20 antibody (e.g., obinutuzumab or rituximab) variant has been modified to increase or decrease the extent to which the bispecific antibody is glycosylated. Addition or deletion of glycosylation sites to an anti-CD20 antibody (e.g., obinutuzumab or rituximab) may be conveniently accomplished by altering the amino acid sequence such that one or more glycosylation sites is created or removed.

Where the bispecific antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15: 26-32 (1997). The oligosaccharide may include various carbohydrates, e.g., mannose, N-acetyl glucosamine (GlcNAc), galactose, and sialic acid, as well as a fucose attached to a

GlcNAc in the “stem” of the biantennary oligosaccharide structure. In some embodiments, modifications of the oligosaccharide in an antibody of the invention may be made in order to create antibody variants with certain improved properties.

In some instances, the anti-CD20 antibody (e.g., obinutuzumab or rituximab) variant has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Patent Publication Nos. US 2003/0157108 (Presta, L.); US 2004/0093621 (Kyowa Hakko Kogyo Co., Ltd). 35 Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336: 1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249: 533-545 (1986); U.S. Pat. Appl. No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4): 680-688 (2006); and WO 2003/085107).

In view of the above, in some instances, the methods of the invention involve administering to the subject in the context of a fractionated, dose-escalation dosing regimen an anti-CD20 antibody (e.g., obinutuzumab or rituximab) variant that comprises an aglycosylation site mutation. In some instances, the aglycosylation site mutation reduces effector function of the bispecific antibody. In some instances, the aglycosylation site mutation is a substitution mutation. In some instances, the bispecific antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G (EU numbering). In some instances, the substitution mutation is at amino acid residue N297 (EU numbering). In a preferred embodiment, the substitution mutation is N297A (EU numbering).

In other instances, bispecific antibody variants with bisected oligosaccharides are used in accordance with the methods of the invention, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### (iii) Fc Region Variants I

n some instances, an anti-CD20 antibody (e.g., obinutuzumab or rituximab) variant that has one or more amino acid modifications introduced into the Fc region (i.e., an Fc region variant (see e.g., US 2012/0251531)) of the bispecific antibody may be administered to a subject having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) in accordance with the methods of the invention. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the bispecific Fc region antibody variant possesses some but not all effector functions, which makes it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express FcyRIII only, whereas monocytes express FcyRI, FcyRI and FcyRIII. FcR expression on hematopoietic cells is summarized in Table 3 on page 464 of Ravetch and Kinet, Annu. Rev. Immunol. 9: 457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83: 7059-7063 (1986)) and Hellstrom, I et al., Proc. Nat'l Acad. Sci. USA 82: 1499-1502 (1985); U.S. Pat. No. 5,821,337 (see Bruggemann, M. et al., J. Exp. Med. 166: 1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95: 652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202: 163 (1996); Cragg, M. S. et al. Blood. 101: 1045-1052 (2003); and Cragg, M. S. and M. J. Glennie Blood. 103: 2738-2743 (2004)). FcRn binding and in vivo clearance/half-life determinations can also be performed using methods known in the art (see, e.g., Petkova, S. B. et al. Int'l. Immunol. 18(12): 1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fcy receptor interface that is formed between the proline 329 of the Fc and tryptophan residues Trp 87 and Trp 110 of FcgRlll (Sondermann et al. Nature. 406, 267-273 (2000)). In certain embodiments, the bispecific antibody comprises at least one further amino acid substitution. In one embodiment, the further amino acid substitution is S228P, E233P, L234A, L235A, L235E, N297A, N297D, or P331S (EU numbering), and still in another embodiment the at least one further amino acid substitution is L234A and L235A (EU numbering) of the human IgG1 Fc region or S228P and L235E (EU numbering) of the human IgG4 Fc region (see e.g., US 2012/0251531), and still in another embodiment the at least one further amino acid substitution is L234A and L235A and P329G (EU numbering) of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, the anti-CD20 antibody (e.g., obinutuzumab or rituximab) comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117: 587 (1976) and Kim et al., J. Immunol. 24: 249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322: 738-40 (1988); U.S. Pat. Nos. 5,648,260; 5,624,821; and WO 94/29351 concerning other examples of Fc region variants.

### (iv) Cysteine Engineered Antibody Variants

In certain embodiments, it may be desirable to create cysteine engineered anti-CD20 antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### (v) Other Antibody Derivatives

In some instances, the anti-CD20 antibody (e.g., obinutuzumab or rituximab) may be modified to contain additional non-proteinaceous moieties that are known in the art and readily available and administered to the subject in accordance with the methods described herein. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### F. Administration

In some instances, the anti-CD20 antibody (e.g., obinutuzumab or rituximab) or compositions thereof, utilized in the methods, uses, assays, and kits described herein can be formulated for administration or administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the anti-CD20 antibody (e.g., obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some instances, the anti-CD20 antibody (e.g., obinutuzumab or rituximab) and any additional therapeutic agent may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In some instances, the anti-CD20 antibody (e.g., obinutuzumab or rituximab) need not be, but is optionally formulated with and/or administered concurrently with, one or more agents currently used to prevent or treat the disorder in question. The effective amount of such other agents depends on the amount of the anti-CD20 antibody (e.g., obinutuzumab or rituximab) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), the appropriate dosage of an anti-CD20 antibody (e.g., obinutuzumab or rituximab) described herein (when used alone or in combination with one or more other additional therapeutic agents) will depend on the type of disease to be treated, the severity and course of the disease, whether the anti-CD20 antibody (e.g., obinutuzumab or rituximab) is administered for preventive or therapeutic purposes, previous therapy, the patient's clinical history and response to the anti-CD20 antibody (e.g., obinutuzumab or rituximab), and the discretion of the attending physician. In some instances, the anti-CD20 antibody (e.g., obinutuzumab or rituximab) is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week, every month, or every two months. An initial higher loading dose followed by one or more lower doses may be administered. However, other dosage regimens may be useful. The progress of this therapy is easily monitored by conventional techniques and assays.

In some instances, an effective amount of the anti-CD20 antibody (e.g., obinutuzumab or rituximab) may be between about 60 mg to about 5000 mg (e.g., between about 60 mg to about 4500 mg, between about 60 mg to about 4000 mg, between about 60 mg to about 3500 mg, between about 60 mg to about 3000 mg, between about 60 mg to about 2500 mg, between about 650 mg to about 2000 mg, between about 60 mg to about 1500 mg, between about 100 mg to about 1500 mg, between about 300 mg to about 1500 mg, between about 500 mg to about 1500 mg, between about 600 mg to about 1400 mg, between about 700 mg to about 1300 mg, between about 800 mg to about 1200 mg, between about 900 mg to about 1100 mg, between about 950 mg to about 1050 mg, between about 975 mg to about 1025 mg, or between about 990 mg to about 1010 mg, e.g., about 1000 mg±5 mg, about 1000±2.5 mg, about 1000±1.0 mg, about 1000±0.5 mg, about 1000±0.2 mg, or about 1000±0.1 mg). In some instances, the methods include administering to the individual the anti-CD20 antibody (e.g., obinutuzumab or rituximab) at about 1000 mg (e.g., a fixed dose of about 1000 mg).

In some aspects, the effective amount of the anti-CD20 antibody (e.g., rituximab or obinutuzumab) is a dose of between about 250 mg/m² to about 500 mg/m² (e.g., between about 250 mg/m² to about 450 mg/m², e.g., between about 250 mg/m² to about 400 mg/m², e.g., between about 300 mg/m² to about 400 mg/m², e.g., between about 325 mg/m² to about 400 mg/m², e.g., between about 350 mg/m² to about 400 mg/m², .g., between about 350 mg/m² to about 375 mg/m², e.g., about 375±2 mg/m^{2, about} 375±1 mg/m², about 375±0.5 mg/m², about 375±0.2 mg/m², or about 375±0.1 mg/m², e.g., about 375 mg/m²). In some aspects, the effective amount of the anti-CD20 antibody (e.g., rituximab or obinutuzumab) is a dose of about 375 mg/m².

In some instances, the anti-CD20 antibody (e.g., obinutuzumab or rituximab) is administered to the individual (e.g., a human) at 1000 mg intravenously on day 1, 8, and 15 of Cycle 1, 1000 mg on day 1 of Cycles 2-6 or Cycles 2-8, and then 1000 mg every 2 months for up to 2 years. In some instances, the anti-CD20 antibody (e.g., rituximab or obinutuzumab) is administered to the individual (e.g., a human) at 375 mg/m² intravenously on day 1 of Cycles 1-8. The dose may be administered as a single dose or as multiple doses (e.g., 2, 3, 4, 5, 6, 7, or more than 7 doses), such as infusions. In some instances, the anti-CD20 antibody (e.g., obinutuzumab or rituximab) administered to the individual (e.g., a human) may be administered alone or in combination with an additional therapeutic agent described herein (e.g., CHOP), in four to six doses. The dose of the antibody administered in a combination treatment may be reduced as compared to a single treatment. The progress of this therapy is easily monitored by conventional techniques.

In one instance, the anti-CD20 antibody (e.g., obinutuzumab or rituximab) is administered as a monotherapy to the individual to treat a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)). In other instances, the anti-CD20 antibody (e.g., obinutuzumab or rituximab) is administered as a combination therapy, as described herein, to the individual to treat a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the anti-CD20 antibody (e.g., obinutuzumab or rituximab) is administered as a combination therapy with CHOP. In some instances, the anti-CD20 antibody (e.g., obinutuzumab or rituximab) is administered to the individual (e.g., a human) at 1000 mg intravenously on day 1, 8, and 15 of Cycle 1, 1000 mg on day 1 of Cycles 2-6 or Cycles 2-8, and then 1000 mg every 2 months for up to 2 years and CHOP is administered for 6 or 8 cycles at the following doses: cyclophosphamide 750 mg/m² IV (Day 1); doxorubicin 50 mg/m² IV (Day 1); vincristine 1.4 mg/m² IV (Day 1, maximum 2.0 mg); and prednisone 100 mg/day orally (Days 1-5). In some instances, the anti-CD20 antibody (e.g., rituximab or obinutuzumab) is administered to the individual (e.g., a human) at 375 mg/m² intravenously on day 1 of Cycles 1-8 and CHOP is administered for 6 or 8 cycles at the following doses: cyclophosphamide 750 mg/m² IV (Day 1); doxorubicin 50 mg/m² IV (Day 1); vincristine 1.4 mg/m² IV (Day 1, maximum 2.0 mg); and prednisone 100 mg/day orally (Days 1-5).

### G. Indications

The methods and compositions described herein are useful for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) by administering an anti-CD20 antibody (e.g., obinutuzumab or rituximab). In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a follicular lymphoma (FL). In some instances, the lymphoma may be a chronic lymphocytic leukemia (CLL). In some instances, the lymphoma may be a CD20-positive lymphoma. In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has not been previously treated for the lymphoma (treatment naïve). For example, in some instances, the individual having a lymphoma has not previously received an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) has previously received treatment for the lymphoma. In some instances, the individual having a lymphoma has previously received treatment including an anti-CD20 antibody (e.g., obinutuzumab or rituximab)).

### H. Combination Therapies

In any of the methods or uses herein, the anti-CD20 antibody (e.g., obinutuzumab or rituximab) may be administered in combination with an effective amount of one or more additional therapeutic agents. Suitable additional therapeutic agents include, for example, an anti-neoplastic agent, a chemotherapeutic agent, a growth inhibitory agent, a cytotoxic agent, a radiotherapy, or combinations thereof.

In some instances, the methods further involve administering to the patient an effective amount of one or more additional therapeutic agents. In some instances, the additional therapeutic agent is selected from the group consisting of a cytotoxic agent, a chemotherapeutic agent, a growth-inhibitory agent, a radiation therapy agent, an anti-angiogenic agent, and combinations thereof. In some instances, an anti-CD20 antibody (e.g., obinutuzumab or rituximab) may be administered in conjunction with a chemotherapy or chemotherapeutic agent. In some instances, an anti-CD20 antibody (e.g., obinutuzumab or rituximab) may be administered in conjunction with a radiation therapy agent. In some instances, an anti-CD20 antibody (e.g., obinutuzumab or rituximab) may be administered in conjunction with a targeted therapy or targeted therapeutic agent. In some instances, an anti-CD20 antibody (e.g., obinutuzumab or rituximab) may be administered in conjunction with an immunotherapy or immunotherapeutic agent, for example a monoclonal antibody. In some instances, the additional therapeutic agent is an agonist directed against an activating co-stimulatory molecule. In some instances, the additional therapeutic agent is an antagonist directed against an inhibitory co-stimulatory molecule.

Such combination therapies noted above encompass combined administration (where two or more therapeutic agents are included in the same or separate formulations), and separate administration, in which case, administration of an anti-CD20 antibody (e.g., obinutuzumab or rituximab) can occur prior to, simultaneously, and/or following, administration of the additional therapeutic agent or agents. In one instance, administration of an anti-CD20 antibody (e.g., obinutuzumab or rituximab) and administration of an additional therapeutic agent occur within about one month, or within about one, two or three weeks, or within about one, two, three, four, five, or six days, of each other.

In some instances, an anti-CD20 antibody (e.g., obinutuzumab or rituximab) may be administered in conjunction with a CHOP chemotherapy or with variants of a CHOP chemotherapy (e.g., a CHOEP chemotherapy, a CHOP-14 chemotherapy, or an ACVBP chemotherapy.

### V. Pharmaceutical Compositions and Formulations

Pharmaceutical compositions and formulations as described herein can be prepared by mixing the active ingredient(s) (e.g., an anti-CD20 antibody) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®; Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases. It is understood that any of the above pharmaceutical compositions or formulations may include an immunoconjugate described herein in place of, or in addition to, an anti-CD20 antibody.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The compositions and formulations herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, for example, by filtration through sterile filtration membranes.

### VI. Articles of Manufacture and Kits

In another aspect of the invention, an article of manufacture or kit containing materials useful for the treatment, prevention, and/or diagnosis of patients is provided.

In some instances, such articles of manufacture or kits can be used to identify a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who may benefit from a treatment comprising an anti-CD20 antibody (e.g., obinutuzumab or rituximab). Such articles of manufacture or kits may include (a) reagents for determining a macrophage biomarker in a sample from the individual and (b) instructions for using the reagents to identify a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who may benefit from a treatment comprising an anti-CD20 antibody (e.g., obinutuzumab or rituximab). Such articles of manufacture or kits may include (a) reagents for determining a Th2 biomarker in a sample from the individual and (b) instructions for using the reagents to identify a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who may benefit from a treatment comprising an anti-CD20 antibody (e.g., obinutuzumab or rituximab).

In some instances, such articles of manufacture or kits include an anti-CD20 antibody (e.g., obinutuzumab or rituximab) for treating a patient with a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)) who may benefit from a treatment comprising an anti-CD20 antibody (e.g., obinutuzumab or rituximab). In some instances, the article of manufacture or kit includes (a) an anti-CD20 antibody (e.g., obinutuzumab or rituximab) and (b) a package insert including instructions for administration of the anti-CD20 antibody (e.g., obinutuzumab or rituximab) to a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) or a marginal zone lymphoma (e.g., an extranodal, nodal, or splenic marginal zone lymphoma)), wherein, prior to treatment, the macrophage biomarker in a sample from the patient has been determined to be above a reference macrophage biomarker amount or level. In some instances, the article of manufacture or kit includes (a) an anti-CD20 antibody (e.g., obinutuzumab or rituximab) and (b) a package insert including instructions for administration of the anti-CD20 antibody (e.g., obinutuzumab or rituximab) to a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) ora marginal zone lymphoma (e.g., an extranodal, nodal, or splenic marginal zone lymphoma)), wherein, prior to treatment, the Th2 biomarker in a sample from the patient has been determined to be above a reference Th2 biomarker amount or level.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, florescent, or radioisotope label.

In some instances, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of embodiments. In one instance, the article of manufacture or kit includes a container, a label on said container, and a composition contained within said container, wherein the composition includes one or more polynucleotides that hybridize to a complement of a gene described herein under stringent conditions, and the label on said container indicates that the composition can be used to evaluate the presence or level of the gene described herein in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

For protein-based articles of manufacture or kits, the article of manufacture or kit can include, for example, an antibody that binds any of the genes described herein. The article of manufacture or kit can further include components necessary for detecting the detectable label (e.g., an enzyme or a substrate). In some instances, the antibody is conjugated to an enzyme (e.g., horseradish peroxidase (HRP)). In some instances, the antibody is conjugated to a fluorophore. The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### VII. EXAMPLES

The following is an example of the methods of the invention. It is understood that various other embodiments may be practiced, given the general description provided above.

### Example 1. Marker Gene-Derived Macrophage Biomarker-Defined Subsets of DLBCL Patients aAre MoreResponsive to Anti-CD20 Antibody Treatment

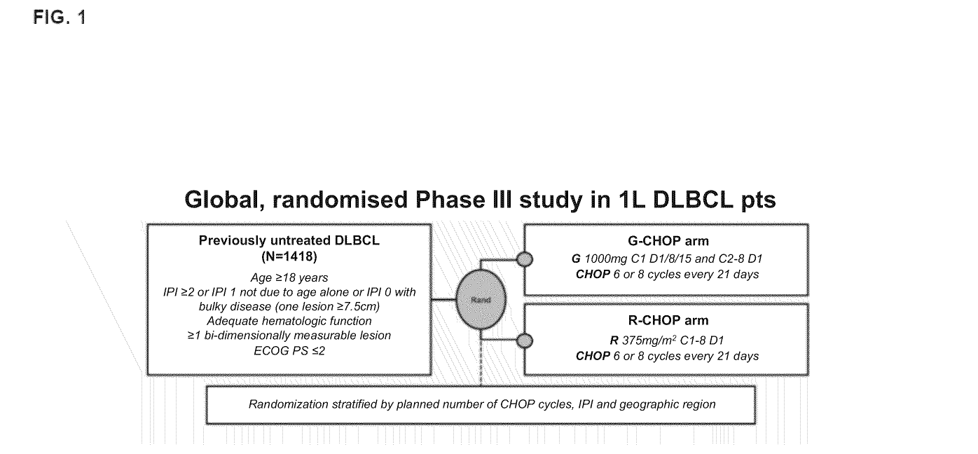

GOYA trial (Clinical Trial ID No. NCT01287741) is a large, open-label, randomized, Phase III clinical trial that compared the efficacy and safety of obinutuzumab in combination with cyclophosphamide, doxorubicin, vincristine, and prednisolone or prednisone (CHOP) chemotherapy versus rituximab 30 (MabThera/Rituxan) with CHOP in previously untreated participants with cluster of differentiation 20 (CD20)-positive diffuse large B-cell lymphoma (DLBCL) (FIG. 1). MAIN clinical trial (Clinical Trial ID No. NCT00486759) is a multicenter, randomized, double-blind, placebo-controlled Phase III trial that compared the efficacy of bevacizumab in combination with rituximab and CHOP (R-CHOP+Bevacizumab) versus rituximab and CHOP (R-CHOP) in previously untreated patients with CD20-positive DLBCL. The objective of this analysis was to assess clinical outcomes in subgroups defined by a macrophage biomarker that dichotomized patients into a high and low macrophage biomarker subset.

### Data set

Biopsy samples, clinical outcomes (overall survival (OS), progression-free survival (PFS)), and high-through transcriptome sequencing data were available for 553 patients from the GOYA trial (NCT01287741; ITT=1418; obinutuzumab (G)-CHOP versus rituximab (R)-CHOP). Data cut-off occurred on January 31, 2018 with a median follow-up of 3.9 years.

Biopsy samples, clinical outcomes (OS and PFS), and high-through transcriptome sequencing data were available for patients from the MAIN clinical trial (Clinical Trial ID No. NCT00486759).

### RNA-Seq Processing

Applying high-throughput transcriptome sequencing quality control was performed. Adapters were trimmed using ea-utils function fastq-mcf (parameters: --max-ns 4 --qual-mean 25 -H -p 5 -q 7 -I 25 -I 25). Trimmed reads were aligned to human genome reference GRCh38 using GSNAP version 2013-10-10 (parameters: -M 2 -n 10 -B 2 -i 1 -N 1 -w 200000 -E 1 --pairmax-rna=200000 --clip-overlap). Leveraging RNA-Seq pipeline scripts, exonic gene counts were determined for each sample.

Genes and samples were filtered using an internal RNA-Seq processing package, rnaseqTools (parameters: default). To account for sequencing variability, counts were normalized to transcripts per million (TPM). From 553 samples from the GOYA trial, a total of 538 passed quality control and were used in downstream analyses. An additional 66 samples from R-CHOP treated patients from the MAIN clinical trial were combined with the 538 samples of the GOYA trial and used in a separate downstream analysis.

### Cell Composition Estimation

Multiple bulk RNA-Seq deconvolution approaches were applied to the final gene by sample count matrix in order to estimate the cellular composition of a sample. In particular, a marker gene (xCell) based approach (see, e.g., Aran et al. Genome Biol. 18(1): 220 (2017)) was used to estimate the cellular composition of a bulk RNA-Seq patient sample. The marker gene method identified the relative composition of numerous cell types, including macrophages, M1 macrophages, M2 macrophages, mast cells, and memory B cells, within/across samples. In particular, the expression of genes within a signature, e.g., M1 macrophages are estimated and statistically tested for enrichment within a sample.

The marker gene approach, xCell, was used to build signatures derived from a compendium of six sequencing and microarray sources: FANTOM5 project (see, e.g., Lizio M, et al. Genome Biol. 16: 22 (2015)), the ENCODE project (see, e.g., ENCODE Project Consortium. Nature. 489(7414): 57-74 (2012)), the Blueprint project (see, e.g., Fernández et al. Cell Syst. 3(5): 491-495.e5 (2016)), the IRIS project (see, e.g., Abbas et al. Genes Immun. 6(4): 319-31 (2005)), the Novershtern et al. study (Novershtern et al. Cell. 144(2): 296-309 (2011)), and the Human Primary Cells Atlas (HPCA) (see, e.g., Uhlén. Science. 347(6220): 1260419 (2015)). Details of genes included in the various signatures are listed in Table 1. The R package immunedeconv (see, e.g., Sturm et al. Bioinformatics. 35(14): i436-i445 (2019)), which serves as a unified interface to multiple immune deconvolution methods, was used to apply xCell.

The estimated percentage of M1 macrophages present in the patient samples as determined by xCell had a range from 0% to about 15.477%, a first quartile of about 2.896%, a median of about 5.171%, and a third quartile of about 7.951%.

### Statistical Analysis

### Biomarker Evaluable Population Comparison to ITT

Initial analysis was performed to ensure the biomarker evaluable population was characteristic of the ITT population. Baseline clinical variables were determined to be similar to the ITT population of the GOYA trial (Table 7).

TABLE 7
GOYA BEP versus ITT

| n (%) | GOYA BEP | | GOYA ITT | |
|---|---|---|---|---|
| Number of evaluable | 499 | | 1418 | |
| patients | | | | |
| Median age (range), years | 64 | (18-86) | 62 | (18-86) |

| | | |
|---|---|---|
| ECOG PS | 445 (89)/53 (11) | 1231 (87)/186 (13) |
| 0-1/2-3 | | |

IPI score

| | | |
|---|---|---|
| Low/Low-intermediate | 102 (20)/171 (34) | 283 (20)/502 (35) |
| High intermediate/High | 146 (29)/80 (16) | 413 (29)/220 (16) |

LDH

| | | |
|---|---|---|
| Elevated/normal | 283 (57)/215 (43) | 816 (58)/597 (42) |

Arm

| G-CHOP (6 cycles/8 cycles) | 171 (34)/76 (15) | | 523 (37)/183 (13) | |
|---|---|---|---|---|
| R-CHOP (6 cycles/8 cycles) | 193 (39)/59 (12) | | 526 (37)/186 (13) | |
| BBMI, number of evaluable | 497 | | 1408 | |
| patients | | | | |
| Mean (SD), kg/m² | 26 | (5) | 25 | (5) |
| COO, number of evaluable | 482 | | 933 | |
| patients | | | | |
| GCB | 272 | (56) | 540 | (58) |

| | | |
|---|---|---|
| ABC/unclassified | 126 (26)/84 (17) | 234 (25)/159 (17) |

ABC, activated B-cell-like;
BBMI, baseline body mass index;
BEP, biomarker evaluable population;
GCB, germinal center B-cell-like;
ITT, intent-to-treat;
LDH, lactate dehydrogenase

### Survival Analysis

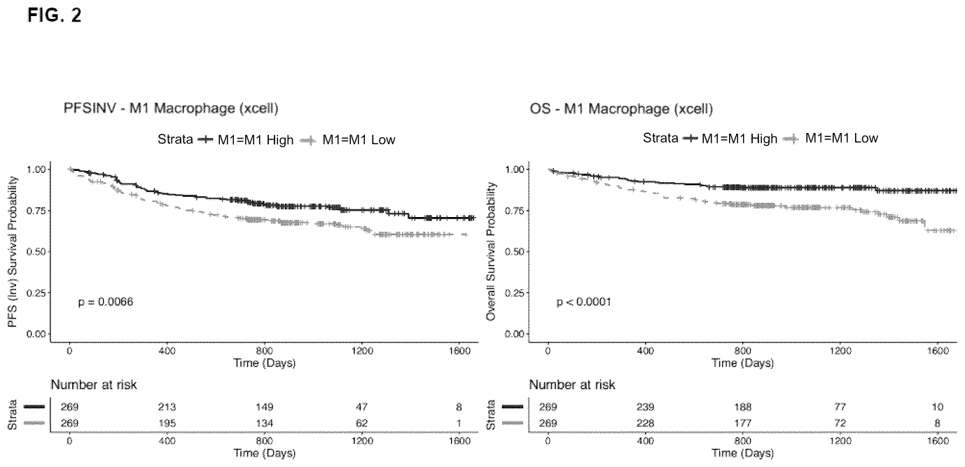

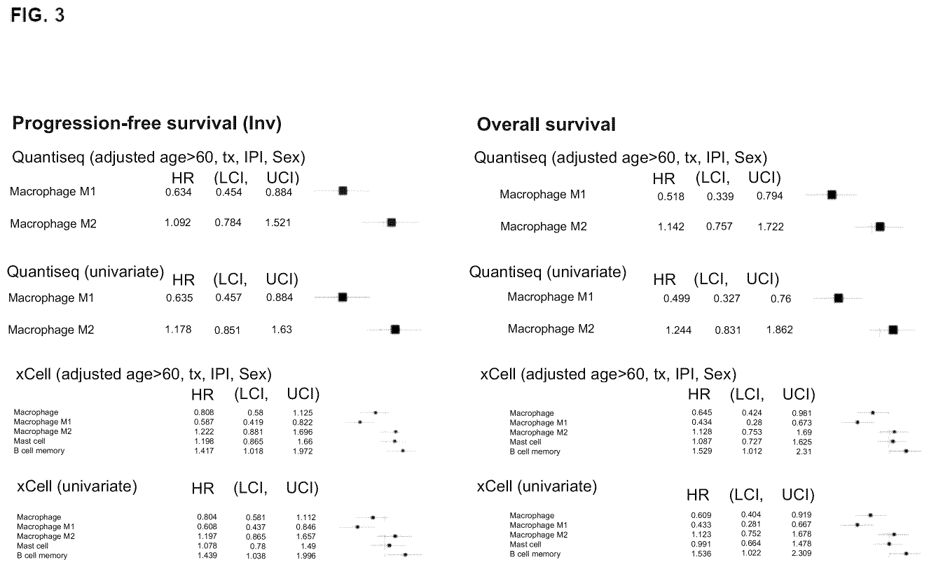

Following estimation of gene signature values employing xCell to our bulk RNA-Seq patient samples from the GOYA trial, a median cutoff (5.171%) was applied to dichotomize patients into high and low biomarker subsets. Kaplan-Meier curves of the associated progression-free survival and overall survival for M1 signature as estimated by xCell are plotted in FIG. 2. Forest plots of hazard ratios and 95% confidence intervals are presented in FIG. 3 on PFS and OS for macrophages, M1 macrophages, M2 macrophages, mast cells, and memory B cells. Multivariate models are adjusted for age (>60), treatment (obinutuzumab versus rituximab), international prognostic index (IPI), and sex. High-biomarker patient subsets dichotomized by the macrophage biomarkers, macrophages and M1 macrophages, had improved OS with treatment including an anti-CD20 antibody, and high-biomarker patient subset dichotomized by the macrophage biomarker, M1 macrophages, had improved PFS with treatment including an anti-CD20 antibody.

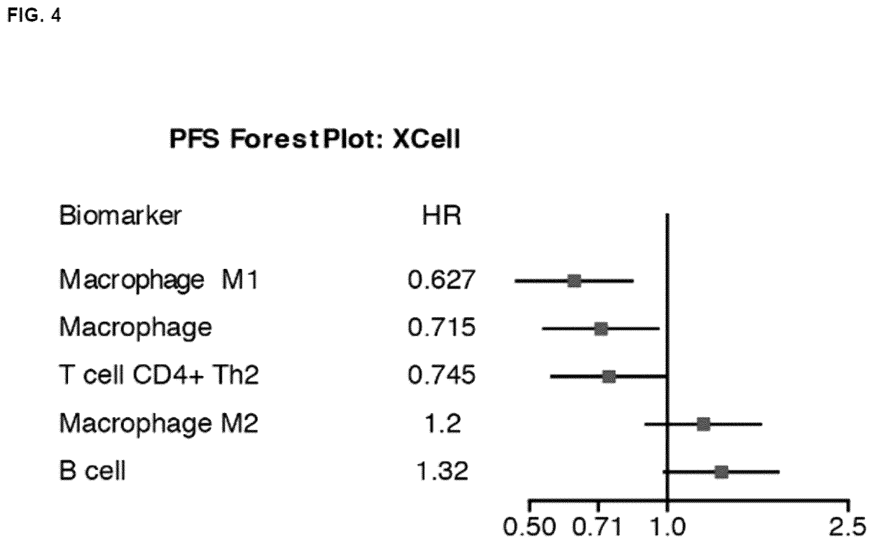

A similar analysis was performed on the combined data of the GOYA and MAIN trials. Of the infiltrating cell types analyzed, the M1 macrophage signature quantified by xCell was most strongly associated with lower risk of progression (PFS: HR, 0.627; 95% CI: 0.465-0.844; 24-month PFS: 80% [M1 high] vs 70% [M1 Low]; FIG. 4) and improved overall survival (OS: HR, 0.527; 95% CI: 0.365-0.762). This prognostic trend was stronger amongst G-treated patients than R-treated patients, consistent with the previous finding that G exhibits higher ADCC versus R (Mossner et al. Blood. 115(22): 4393-402 (2010)). Patients with PFS >24 months had significantly higher levels of M1 macrophage scores tha patients with PFS <24 months. Despite the correlation with delayed disease progression, there was no differential enrichment of M1 macrophages in patients with complete response versus non-responders at end of treatment, or depending on International Prognostic Index. M1 scores did not significantly differ depending on cell of origin, although there was a trend for higher M1 macrophage scores in germinal center B-cell DLBCL. Aside from M1 macrophages, CD4+Th2 cells showed the strongest prognostic trend in DLBCL (PFS; HR, 0.745; 95% CI: 0.553-1.000; FIG. 4). In contrast to M1 macrophages, patients with M2 macrophage infiltration tended to have shorter PFS and OS although relatively low levels were observed for these signatures (FIG. 4). This suggests that lymphoma-infiltrating macrophages more commonly resemble the classically activated M1 polarization phenotype and are linked to prolonged PFS, while alternatively activated M2 macrophages, although their frequency is lower in DLBCL, are associated with shorter PFS. Consistent with previous work showing that programmed death-ligand 1 (PD-L1) levels correlate with a macrophage signature in DLBCL (McCord et al. Blood Adv. 3(4): 531-540 (2019)), M1, but not M2, macrophage infiltration correlated with PD-L1 mRNA expression.

### Example 2. Deconvolution-Derived Macrophage Biomarker-Defined Subset of DLBCL Patients is More Responsive to Anti-CD20 Antibody Treatment

The GOYA trial (Clinical Trial ID No. NCT01287741) and MAIN clinical trial (Clinical Trial ID No. NCT00486759) data was collected and processed as described above in Example 1.

### Cell Composition Estimation

Multiple bulk RNA-Seq deconvolution approaches were applied to the final gene by sample count matrix in order to estimate the cellular composition of a sample. In particular, a deconvolution (quanTlseq) based approach (see, e.g., Finotello et al. Genome Med. 11(1): 34 (2019)) was used to estimate the cellular composition of a bulk RNA-Seq patient sample. The deconvolution method identified the relative composition of numerous cell types, including M1 macrophages and M2 macrophages, within/across samples. In particular, the deconvolution based approach articulates the problem as a system of equations solved using constrained least squares for a set of signatures.

The deconvolution based approach leveraged blood and tumor samples using simulated, flow cytometry, and immunohistochemistry data to apply quanTlseq. Genes used in the deconvolution signature methodology are listed in Table 3 and Table 4. The R package immunedeconv (see, e.g., Sturm et al. Bioinformatics. 35(14): i436-i445 (2019)), which serves as a unified interface to multiple immune deconvolution methods, was used to apply quanTlseq.

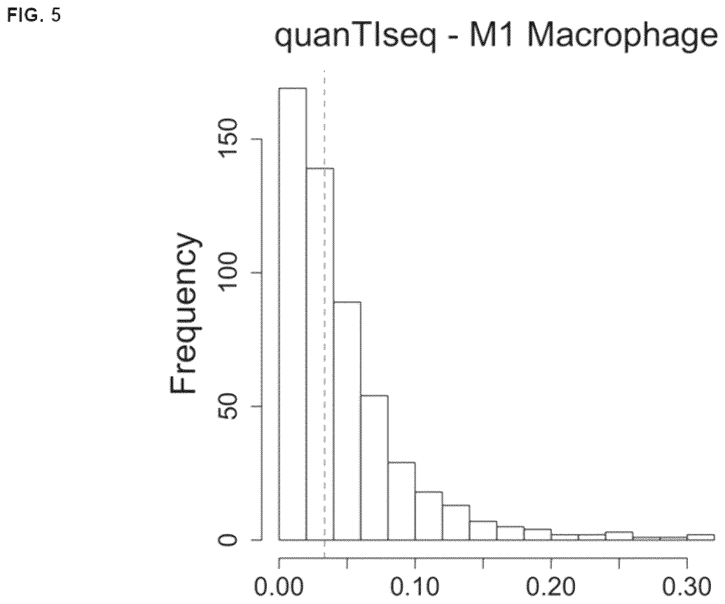

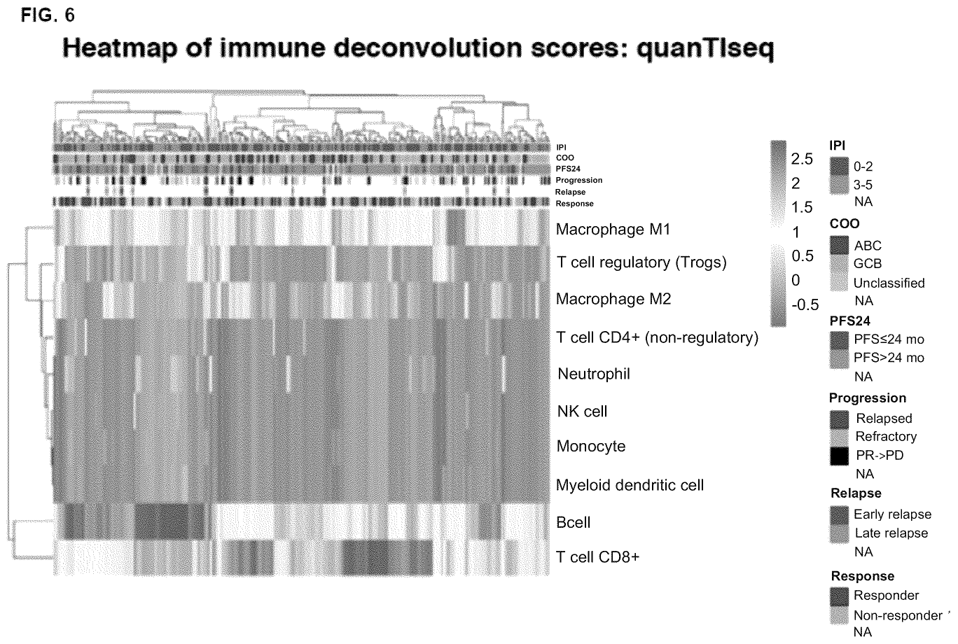

The estimated percentage of M1 macrophages present in the GOYA patient samples as determined by quanTlseq had a range from 0% to about 30.678%, a first quartile of about 1.664%, a median of about 3.346%, a mean of about 4.738% and a third quartile of about 6.178% (FIG. 5). The extent of lymphoma microenvironment heterogeneity highlighted by the deconvolution analyses was consistent with previous studies (FIG. 6).

### Statistical Analysis

### Biomarker Evaluable Population Comparison to ITT

Initial analysis was performed to ensure the biomarker evaluable population was characteristic of the ITT population. Baseline clinical variables were determined to be similar to the ITT population for the GOYA trial (Table 7).

### Survival Analysis

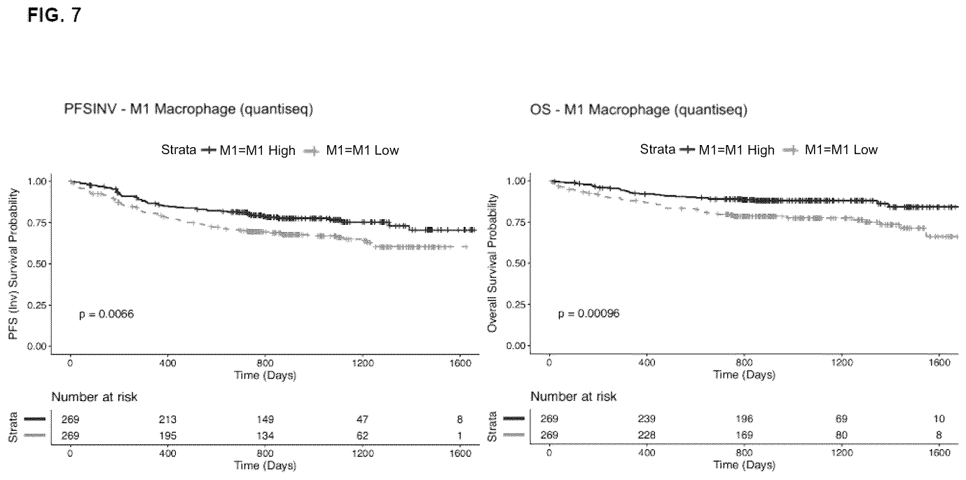

Following estimation of gene signature values employing quanTlseq to our bulk RNA-Seq patient samples from the GOYA trial, a median cutoff (3.346%) was applied to dichotomize patients into high and low biomarker subsets. Kaplan-Meier curves of the associated progression-free survival and overall survival for M1 signature as estimated by quanTlseq are plotted in FIG. 7. Forest plots of hazard ratios and 95% confidence intervals for PFS and OS are presented in FIG. 3 for M1 macrophages and M2 macrophages. Multivariate models are adjusted for age (>60), treatment (obinutuzumab versus rituximab), IPI, and sex. High biomarker patient subsets dichotomized by the macrophage biomarker, M1 macrophages, had improved OS and PFS with treatment including an anti-CD20 antibody.

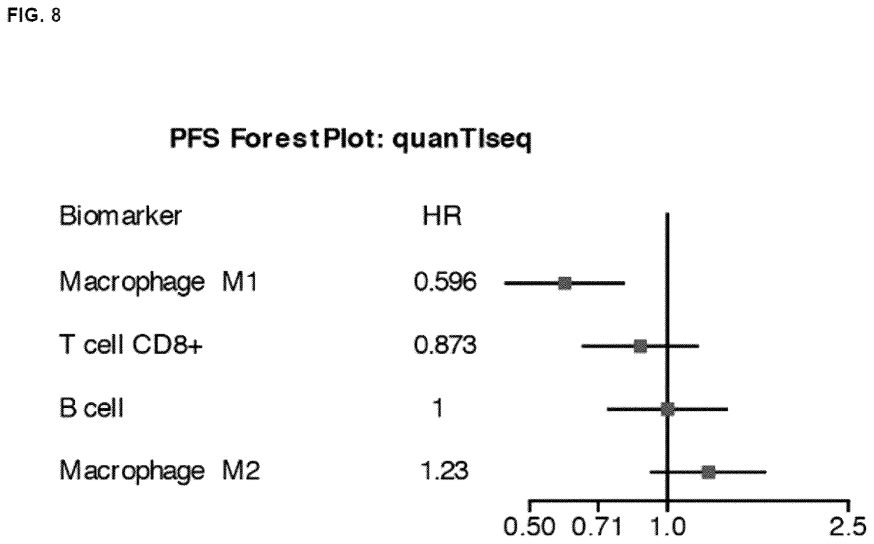

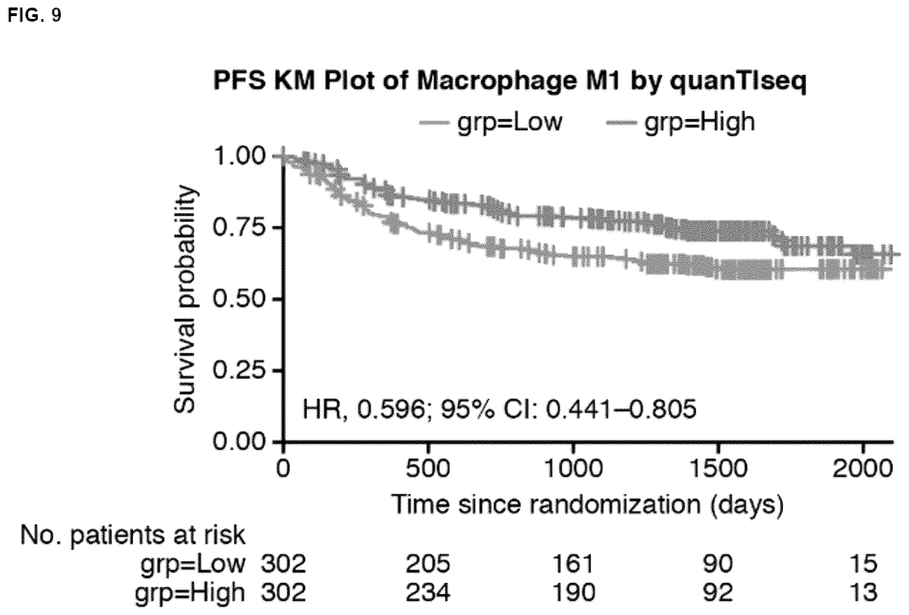

A similar analysis was performed on the combined data of the GOYA and MAIN trials. Of the infiltrating cell types analyzed, the M1 macrophage signature quantified by quanTlseq was most strongly associated with lower risk of progression (PFS: HR, 0.596; 95% CI: 0.441-0.805; 24-month PFS: 82% [M1 high] vs 68% [M1 low]; FIGS. 8 and 9) and improved overall survival (OS: HR, 0.465; 95% CI: 0.318-0.679). This prognostic trend was stronger amongst G-treated patients than R-treated patients, consistent with the previous finding that G exhibits higher ADCC versus R (Mossner et al. Blood. 115(22): 4393-402 (2010)).

Patients with PFS >24 months had significantly higher levels of M1 macrophage scores tha patients with PFS <24 months. Despite the correlation with delayed disease progression, there was no differential enrichment of M1 macrophages in patients with complete response versus non-responders at end of treatment, or depending on International Prognostic Index. M1 scores did not significantly differ depending on cell of origin, although there was a trend for higher M1 macrophage scores in germinal center B-cell DLBCL. In contrast to M1 macrophages, patients with M2 macrophage infiltration tended to have shorter PFS and OS although relatively low levels were observed for these signatures (FIG. 8). This suggests that lymphoma-infiltrating macrophages more commonly resemble the classically activated M1 polarization phenotype and are linked to prolonged PFS, while alternatively activated M2 macrophages, although their frequency is lower in DLBCL, are associated with shorter PFS. Consistent with previous work showing that programmed death-ligand 1 (PD-L1) levels correlate with a macrophage signature in DLBCL (McCord et al. Blood Adv. 3(4): 531-540 (2019)), M1, but not M2, macrophage infiltration correlated with PD-L1 mRNA expression. M1 enrichment 5 was highly correlated with CD8+ T cell signatures (including central and effector memory CD8+ T cells) in DLBCL.

### VIII. Other Embodiments

Some embodiments of the technology described herein can be defined according to any of the following numbered embodiments:

1. A method of identifying, diagnosing, and/or predicting whether a patient having a lymphoma may benefit from a treatment comprising an anti-CD20 antibody, the method comprising measuring a macrophage biomarker in a sample from the patient, wherein an amount or level of the macrophage biomarker in the sample that is above a reference macrophage biomarker amount or level identifies, diagnoses, and/or predicts the patient as one who may benefit from a treatment comprising an anti-CD20 antibody.

2. A method of selecting a therapy for a patient having a lymphoma, the method comprising measuring a macrophage biomarker in a sample from the patient, wherein an amount or level of the macrophage biomarker in the sample that is above a reference macrophage biomarker amount or level identifies the patient as one who may benefit from a treatment comprising an anti-CD20 antibody.

3. The method of embodiment 1 or 2, wherein the patient has a macrophage biomarker in the sample that is above a reference macrophage biomarker amount or level, and the method further comprises administering to the patient an effective amount of an anti-CD20 antibody.

4. A method of treating a patient having a lymphoma, the method comprising:

(a) measuring a macrophage biomarker in a sample from the patient, wherein the amount or level of the macrophage biomarker in the sample is above a reference macrophage biomarker amount or level, and

(b) administering an effective amount of an anti-CD20 antibody to the patient based on the macrophage biomarker measured in step (a).

5. A method of treating a patient having a lymphoma, the method comprising administering to the patient an effective amount of an anti-CD20 antibody, wherein prior to treatment the amount or level of a macrophage biomarker in a sample from the patient has been determined to be above a reference macrophage biomarker amount or level.

6. A method of treating a patient having a lymphoma and having an amount or level of a macrophage biomarker in a sample from the patient that is above a reference macrophage biomarker amount or level comprising administering to the patient an effective amount of an anti-CD20 antibody.

7. The method of any one of embodiments 1-6, wherein the reference macrophage biomarker amount or level is a pre-assigned macrophage biomarker amount or level.

8. The method of any one of embodiments 1-7, wherein the reference macrophage biomarker amount or level is an amount or level of a macrophage biomarker in a reference population.

9. The method of embodiment 8, wherein the amount or level of the macrophage biomarker in a reference population is a median amount or level of the macrophage biomarker of the reference population.

10. The method of embodiment 8, wherein the reference macrophage biomarker amount or level is an amount or level of a macrophage biomarker that is at the 25th percentile of the reference population.

11. The method of embodiment 8, wherein the reference macrophage biomarker amount or level is an amount or level of a macrophage biomarker that is at the 50th percentile of the reference population.

12. The method of embodiment 8, wherein the reference macrophage biomarker amount or level is an amount or level of a macrophage biomarker that is at the 75th percentile of the reference population.

13. The method of any one of embodiments 8-12, wherein the reference population is a population of patients having the lymphoma.

14. The method of embodiment 13, wherein the population of patients having the lymphoma was previously treated with an anti-CD20 antibody.

15. The method of embodiment 14, wherein the reference macrophage biomarker amount or level is the amount or level of macrophage biomarker of the reference population prior to initiating treatment with the anti-CD20 antibody.

16. The method of embodiment 14 or 15, wherein the reference macrophage biomarker amount or level significantly separates the reference population into a first set of patients who have benefitted from the treatment with the anti-CD20 antibody and a second set of patients who have not benefitted from the treatment with the anti-CD20 antibody.

17. The method of any one of embodiments 1-16, wherein the reference macrophage biomarker amount or level is an amount of macrophages as measured by gene expression.

18. The method of embodiment 17, wherein the amount of macrophages is between about 0% to about 30.7%.

19. The method of any one of embodiments 1-3, wherein the benefit is an extension of progression-free survival (PFS).

20. The method of any one of embodiments 1-3, wherein the benefit is an increase in overall survival (OS).

21. The method of any one of embodiments 4-6, further comprising achieving an improvement of PFS or OS.

22. The method of any one of embodiments 1-21, wherein the macrophage biomarker is an average of M1 macrophage gene signature set scores of one or more M1 macrophage gene signature sets.

23. The method of embodiment 22, wherein each M1 macrophage gene signature set score is an average of the expression level of one or more genes of an M1 macrophage gene signature set.

24. The method of embodiment 23, wherein each M1 macrophage gene signature set score is an verage of the normalized expression level of one or more genes of an M1 macrophage gene signature set.

25. The method of embodiment 23 or 24, wherein the one or more M1 macrophage gene signature sets are:

(a) ACP2, ABCD1, C1QA, FDX1, CCL22, CD163, SCAMP2, ADAMDEC1, ARL8B, and HAMP;

(b) ACP2, ABCD1, FDX1, CCL8, CCL22, CD163, ADAMDEC1, TREM2, and HAMP;

(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;

(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB31L1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;

(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or

(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

26. The method of any one of embodiments 1-21, wherein the macrophage biomarker is a gene expression value.

27. The method of embodiment 26, wherein the gene expression value is a median gene expression value.

28. The method of embodiment 26 or 27, wherein the gene expression value is measured using a gene signature matrix.

29. The method of embodiment 28, wherein the gene signature matrix comprises the following genes:

(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1,

SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or (b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

30. The method of embodiment 29, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

31. The method of any one of embodiments 28-30, wherein the gene signature matrix is used to determine a number of M1 macrophages.

32. The method of any one of embodiments 1-31, wherein the macrophage biomarker is an amount of M1 macrophages.

33. The method of embodiment 32, wherein the amount of M1 macrophages is measured directly or indirectly.

34. The method of embodiment 33, wherein the amount of M1 macrophages is measured directly using flow cytometry, spatial transcriptomics, spatial proteomics, or combination thereof.

35. The method of embodiment 33, wherein the amount of M1 macrophages is measured indirectly using nucleic acid or protein.

36. The method of embodiment 35, wherein the nucleic acid is measured using RNA-seq, RT-qPCR, qPCR, multiplex qPCR or RT-qPCR, microarray analysis, SAGE, MassARRAY technique, ISH, or a combination thereof.

37. The method of embodiment 36, wherein the amount of M1 macrophages is measured using a marker gene approach or a deconvolution approach.

38. The method of embodiment 37, wherein the marker gene approach uses xCell.

39. The method of embodiment 37, wherein the deconvolution approach uses quanTlseq.

40. The method of any one of embodiments 1-21, wherein the macrophage biomarker in the sample from the patient is measured using nucleic acid or protein.

41. The method of embodiment 40, wherein the macrophage biomarker in the sample from the patient is determined using a nucleic acid expression level.

42. The method of embodiment 41, wherein the nucleic acid expression level is determined by RNA-seq, RT-qPCR, qPCR, multiplex qPCR or RT-qPCR, microarray analysis, SAGE, MassARRAY technique, ISH, or a combination thereof.

43. The method of embodiment 41 or 42, wherein the nucleic acid expression level is an mRNA expression level.

44. The method of embodiment 43, wherein the mRNA expression level is determined by RNA-seq.

45. The method of any one of embodiments 1-44, wherein the sample is a tissue sample, tumor sample, whole blood sample, a plasma sample, a serum sample, or a combination thereof.

46. The method of embodiment 45, wherein the sample is a tissue sample.

47. The method of embodiment 46, wherein the tissue sample is a tumor tissue sample.

48. The method of embodiment 47, wherein the tumor tissue sample contains tumor cells, tumor-infiltrating immune cells, stromal cells, normal adjacent tissue (NAT) cells, or a combination thereof.

49. The method of embodiment 47 or 48, wherein the tumor tissue sample is a biopsy.

50. The method of any one of embodiments 45-49, wherein the sample is an archival sample, a fresh sample, or a frozen sample.

51. The method of any one of embodiments 1-50, wherein the lymphoma is an indolent lymphoma.

52. The method of any one of embodiments 1-51, wherein the lymphoma is a B-cell lymphoma.

53. The method of embodiment 52, wherein the B-cell lymphoma is a germinal center derived B-cell lymphoma.

54. The method of embodiment 52 or 53, wherein the B-cell lymphoma is a non-Hodgkin lymphoma (NHL).

55. The method of any one of embodiments 1-54, wherein the lymphoma is a diffuse large B-cell lymphoma (DLBCL), a follicular lymphoma (FL), a chronic lymphocytic leukemia (CLL), or a marginal zone lymphoma (MZL).

56. The method of embodiment 55, wherein the lymphoma is a DLBCL.

57. The method of embodiment 56, wherein the DLBCL is a germinal-center B-cell-like (GCB) or activated B-cell-like (ABC) cell-of-origin subgroup of DLBCL.

58. The method of any one of embodiments 1-57, wherein the lymphoma is a CD20-positive lymphoma.

59. The method of any one of embodiments 1-58, wherein the anti-CD20 antibody is a type I anti-CDantibody or a type II anti-CD20 antibody.

60. The method of embodiment 59, wherein the anti-CD20 antibody is a type II anti-CD20 antibody.

61. The method of embodiment 60, wherein the type II anti-CD20 antibody comprises the following complementarity determining regions (CDRs):

(a) a CDR-H1 with an amino acid sequence of SEQ ID NO: 27;

(b) a CDR-H2 with an amino acid sequence of SEQ ID NO: 28;

(c) a CDR-H3 with an amino acid sequence of SEQ ID NO: 3;

(d) a CDR-L1 with an amino acid sequence of SEQ ID NO: 4;

(e) a CDR-L2 with an amino acid sequence of SEQ ID NO: 5; and

(f) a CDR-L3 with an amino acid sequence of SEQ ID NO: 6.

62. The method of embodiment 61, wherein the type II anti-CD20 antibody comprises a VH domain comprising an amino acid sequence of SEQ ID NO: 7 and a VL domain comprising an amino acid sequence of SEQ ID NO: 8.

63. The method of embodiment 62, wherein the type II anti-CD20 antibody is obinutuzumab.

64. The method of embodiment 59, wherein the anti-CD20 antibody is a type I anti-CD20 antibody.

65. The method of embodiment 64, wherein the type I anti-CD20 antibody comprises the following CDRs:

(a) a CDR-H1 with an amino acid sequence of SEQ ID NO: 11;

(b) a CDR-H2 with an amino acid sequence of SEQ ID NO: 12;

(c) a CDR-H3 with an amino acid sequence of SEQ ID NO: 13;

(d) a CDR-L1 with an amino acid sequence of SEQ ID NO: 14;

(e) a CDR-L2 with an amino acid sequence of SEQ ID NO: 15; and

(f) a CDR-L3 with an amino acid sequence of SEQ ID NO: 16.

66. The method of embodiment 65, wherein the type I anti-CD20 antibody comprises a VH domain comprising an amino acid sequence of SEQ ID NO: 25 and a VL domain comprising an amino acid sequence of SEQ ID NO: 26.

67. The method of embodiment 66, wherein the type I anti-CD20 antibody is rituximab.

68. The method of any one of embodiments 3-67, further comprising administering to the patient an effective amount of an additional therapeutic agent.

69. The method of embodiment 68, wherein the additional therapeutic agent is one or more of a chemotherapeutic agent, an anti-neoplastic agent, a growth inhibitory agent, an anti-angiogenic agent, a radiation therapy, a cytotoxic agent, or a combination thereof.

70. The method of embodiment 69, wherein the additional therapeutic agent is a chemotherapeutic agent.

71. The method of embodiment 69 or 70, wherein the chemotherapeutic agent is cyclophosphamide, doxorubicin, vincristine, or prednisone.

72. The method of embodiments 69 or 70, wherein the chemotherapeutic agent is cyclophosphamide, doxorubicin, vincristine, and prednisone.

73. The method of any one of embodiments 1-72, wherein the patient has not been previously treated for the lymphoma.

74. The method of any one of embodiments 1-73, wherein the patient has not been previously administered an anti-CD20 antibody.

75. Use of an anti-CD20 antibody for treating a patient having an amount or level of a macrophage biomarker in a sample from the patient that is above a reference macrophage biomarker amount or level in the manufacture of a medicament for the treatment of a lymphoma.

76. The use of embodiment 75, wherein the reference macrophage biomarker amount or level is a pre-assigned macrophage biomarker amount or level.

77. The use of embodiment 75 or 76, wherein the reference macrophage biomarker amount or level is an amount or level of a macrophage biomarker in a reference population.

78. The use of embodiment 77, wherein the amount or level of the macrophage biomarker in a reference population is a median amount or level of the macrophage biomarker of the reference population.

79. The use of embodiment 77, wherein the reference macrophage biomarker amount or level is an amount or level of a macrophage biomarker that is at the 25th percentile of the reference population.

80. The use of embodiment 77, wherein the reference macrophage biomarker amount or level is an amount or level of a macrophage biomarker that is at the 50th percentile of the reference population.

81. The use of embodiment 77, wherein the reference macrophage biomarker amount or level is an amount or level of a macrophage biomarker that is at the 75th percentile of the reference population.

82. The use of any one of embodiments 77-81, wherein the reference population is a population of patients having the lymphoma.

83. The use of embodiment 82, wherein the population of patients having the lymphoma was previously treated with an anti-CD20 antibody.

84. The use of embodiment 83, wherein the reference macrophage biomarker amount or level is the amount or level of macrophage biomarker of the reference population prior to initiating treatment with the anti-CD20 antibody.

85. The use of embodiment 83 or 84, wherein the reference macrophage biomarker amount or level significantly separates the reference population into a first set of patients who have benefitted from the treatment with the anti-CD20 antibody and a second set of patients who have not benefitted from the treatment with the anti-CD20 antibody.

86. The use of any one of embodiments 75-85, wherein the reference macrophage biomarker amount or level is an amount of macrophages as measured by gene expression.

87. The use of embodiment 86, wherein the amount of macrophages is between about 0% to about 30.7%.

88. The use of embodiment 75, wherein the treatment achieves an improvement of PFS or OS.

89. The use of any one of embodiments 75-88, wherein the macrophage biomarker is an average of M1 macrophage gene signature set scores of one or more M1 macrophage gene signature sets.

90. The use of embodiment 89, wherein each M1 macrophage gene signature set score is an average of the expression level of one or more genes of an M1 macrophage gene signature set.

91. The use of embodiment 90, wherein each M1 macrophage gene signature set score is an average of the normalized expression level of one or more genes of an M1 macrophage gene signature set.

92. The use of embodiment 90 or 91, wherein the one or more M1 macrophage gene signature sets are:

(a) ACP2, ABCD1, C1QA, FDX1, CCL22, CD163, SCAMP2, ADAMDEC1, ARL8B, and HAMP;

(b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;

(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;

(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or

(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

93. The use of any one of embodiments 75-88, wherein the macrophage biomarker is a gene expression value.

94. The use of embodiment 93, wherein the gene expression value is a median gene expression value.

95. The use of embodiment 93 or 94, wherein the gene expression value is measured using a gene signature matrix.

96. The use of embodiment 95, wherein the gene signature matrix comprises the following genes:

(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or

(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

97. The use of embodiment 96, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

98. The use of any one of embodiments 95-97, wherein the gene signature matrix is used to determine a number of M1 macrophages.

99. The use of any one of embodiments 75-98, wherein the macrophage biomarker is an amount of M1 macrophages.

100. The use of embodiment 99, wherein the amount of M1 macrophages is measured directly or indirectly.

101. The use of embodiment 100, wherein the amount of M1 macrophages is measured directly using flow cytometry, spatial transcriptomics, spatial proteomics, or combination thereof.

102. The use of embodiment 100, wherein the amount of M1 macrophages is measured indirectly using nucleic acid or protein.

103. The use of embodiment 102, wherein the nucleic acid is measured using RNA-seq, RT-qPCR, qPCR, multiplex qPCR or RT-qPCR, microarray analysis, SAGE, MassARRAY technique, ISH, or a combination thereof.

104. The use of embodiment 103, wherein the amount of M1 macrophages is measured using a marker gene approach or a deconvolution approach.

105. The use of embodiment 104, wherein the marker gene approach uses xCell.

106. The use of embodiment 104, wherein the deconvolution approach uses quanTlseq.

107. The use of any one of embodiments 75-88, wherein the macrophage biomarker in the sample from the patient is measured using nucleic acid or protein.

108. The use of embodiment 107, wherein the macrophage biomarker in the sample from the patient is determined using a nucleic acid expression level.

109. The use of embodiment 108, wherein the nucleic acid expression level is determined by RNA-seq, RT-qPCR, qPCR, multiplex qPCR or RT-qPCR, microarray analysis, SAGE, MassARRAY technique, ISH, or a combination thereof.

110. The use of embodiment 108 or 109, wherein the nucleic acid expression level is an mRNA expression level.

111. The use of embodiment 110, wherein the mRNA expression level is determined by RNA-seq.

112. The use of any one of embodiments 75-111, wherein the sample is a tissue sample, tumor sample, whole blood sample, a plasma sample, a serum sample, or a combination thereof.

113. The use of embodiment 112, wherein the sample is a tissue sample.

114. The use of embodiment 113, wherein the tissue sample is a tumor tissue sample.

115. The use of embodiment 114, wherein the tumor tissue sample contains tumor cells, tumor-infiltrating immune cells, stromal cells, NAT cells, or a combination thereof.

116. The use of embodiment 114 or 115, wherein the tumor tissue sample is a biopsy.

117. The use of any one of embodiments 112-116, wherein the sample is an archival sample, a fresh sample, or a frozen sample.

118. The use of any one of embodiments 75-117, wherein the lymphoma is an indolent lymphoma.

119. The use of any one of embodiments 75-118, wherein the lymphoma is a B-cell lymphoma.

120. The use of embodiment 118 or 119, wherein the B-cell lymphoma is an NHL.

121. The use of any one of embodiments 75-120, wherein the lymphoma is a DLBCL, an FL, a CLL, or an MZL.

122. The use of embodiment 121, wherein the lymphoma is a DLBCL.

123. The use of embodiment 122, wherein the DLBCL is a GCB or ABC cell-of-origin subgroup of DLBCL.

124. The use of any one of embodiments 75-123, wherein the lymphoma is a CD20-positive lymphoma.

125. The use of any one of embodiments 75-124, wherein the anti-CD20 antibody is a type I anti-CD20 antibody or a type II anti-CD20 antibody.

126. The use of embodiment 125, wherein the anti-CD20 antibody is a type II anti-CD20 antibody.

127. The use of embodiment 126, wherein the type II anti-CD20 antibody comprises the following CDRs:

(a) a CDR-H1 with an amino acid sequence of SEQ ID NO: 27;

(b) a CDR-H2 with an amino acid sequence of SEQ ID NO: 28;

(c) a CDR-H3 with an amino acid sequence of SEQ ID NO: 3;

(d) a CDR-L1 with an amino acid sequence of SEQ ID NO: 4;

(e) a CDR-L2 with an amino acid sequence of SEQ ID NO: 5; and

(f) a CDR-L3 with an amino acid sequence of SEQ ID NO: 6.

128. The use of embodiment 127, wherein the type II anti-CD20 antibody comprises a VH domain comprising an amino acid sequence of SEQ ID NO: 7 and a VL domain comprising an amino acid sequence of SEQ ID NO: 8.

129. The use of embodiment 128, wherein the type II anti-CD20 antibody is obinutuzumab.

130. The use of embodiment 125, wherein the anti-CD20 antibody is a type I anti-CD20 antibody.

131. The use of embodiment 130, wherein the type I anti-CD20 antibody comprises the following CDRs:

(a) a CDR-H1 with an amino acid sequence of SEQ ID NO: 11;

(b) a CDR-H2 with an amino acid sequence of SEQ ID NO: 12;

(c) a CDR-H3 with an amino acid sequence of SEQ ID NO: 13;

(d) a CDR-L1 with an amino acid sequence of SEQ ID NO: 14;

(e) a CDR-L2 with an amino acid sequence of SEQ ID NO: 15; and

(f) a CDR-L3 with an amino acid sequence of SEQ ID NO: 16.

132. The use of embodiment 131, wherein the type I anti-CD20 antibody comprises a VH domain comprising an amino acid sequence of SEQ ID NO: 25 and a VL domain comprising an amino acid sequence of SEQ ID NO: 26.

133. The use of embodiment 132, wherein the type I anti-CD20 antibody is rituximab.

134. The use of any one of embodiments 75-133, wherein the medicament is to be administered to the patient in combination with an effective amount of an additional therapeutic agent.

135. The use of embodiment 134, wherein the additional therapeutic agent is one or more of a chemotherapeutic agent, an anti-neoplastic agent, a growth inhibitory agent, an anti-angiogenic agent, a radiation therapy, a cytotoxic agent, or a combination thereof.

136. The use of embodiment 135, wherein the additional therapeutic agent is a chemotherapeutic agent.

137. The use of embodiment 135 or 136, wherein the chemotherapeutic agent is cyclophosphamide, doxorubicin, vincristine, or prednisone.

138. The use of embodiment 135 or 136, wherein the chemotherapeutic agent is cyclophosphamide, doxorubicin, vincristine, and prednisone.

139. The use of any one of embodiments 75-138, wherein the patient has not been previously treated for the lymphoma.

140. The use of any one of embodiments 75-139, wherein the patient has not been previously administered an anti-CD20 antibody.

141. An anti-CD20 antibody for use in the treatment of a patient having a lymphoma and having an amount or level of a macrophage biomarker in a sample from the patient that is above a reference macrophage biomarker amount or level.

142. The anti-CD20 antibody for use of embodiment 141, wherein the reference macrophage biomarker amount or level is a pre-assigned macrophage biomarker amount or level.

143. The anti-CD20 antibody for use of embodiment 141 or 142, wherein the reference macrophage biomarker amount or level is an amount or level of a macrophage biomarker in a reference population.

144. The anti-CD20 antibody for use of embodiment 143, wherein the amount or level of the macrophage biomarker in a reference population is a median amount or level of the macrophage biomarker of the reference population.

145. The anti-CD20 antibody for use of embodiment 143, wherein the reference macrophage biomarker amount or level is an amount or level of a macrophage biomarker that is at the 25th percentile of the reference population.

146. The anti-CD20 antibody for use of embodiment 143, wherein the reference macrophage biomarker amount or level is an amount or level of a macrophage biomarker that is at the 50th percentile of the reference population.

147. The anti-CD20 antibody for use of embodiment 143, wherein the reference macrophage biomarker amount or level is an amount or level of a macrophage biomarker that is at the 75th percentile of the reference population.

148. The anti-CD20 antibody for use of any one of embodiments 143-147, wherein the reference population is a population of patients having the lymphoma.

149. The anti-CD20 antibody for use of embodiment 148, wherein the population of patients having the lymphoma was previously treated with an anti-CD20 antibody.

150. The anti-CD20 antibody for use of embodiment 149, wherein the reference macrophage biomarker amount or level is the amount or level of macrophage biomarker of the reference population prior to initiating treatment with the anti-CD20 antibody.

151. The anti-CD20 antibody for use of embodiment 149 or 150, wherein the reference macrophage biomarker amount or level significantly separates the reference population into a first set of patients who have benefitted from the treatment with the anti-CD20 antibody and a second set of patients who have not benefitted from the treatment with the anti-CD20 antibody.

152. The anti-CD20 antibody for use of any one of embodiments 141-151, wherein the reference macrophage biomarker amount or level is an amount of macrophages as measured by gene expression.

153. The anti-CD20 antibody for use of embodiment 152, wherein the amount of macrophages is between about 0% to about 30.7%.

154. The anti-CD20 antibody for use of embodiment 141, wherein the treatment achieves an improvement of PFS or OS.

155. The anti-CD20 antibody for use of any one of embodiments 141-154, wherein the macrophage biomarker is an average of M1 macrophage gene signature set scores of one or more M1 macrophage gene signature sets.

156. The anti-CD20 antibody for use of embodiment 155, wherein each M1 macrophage gene signature set score is an average of the expression level of one or more genes of an M1 macrophage gene signature set.

157. The anti-CD20 antibody for use of embodiment 156, wherein each M1 macrophage gene signature set score is an average of the normalized expression level of one or more genes of an M1 macrophage gene signature set.

158. The anti-CD20 antibody for use of embodiment 156 or 157, wherein the one or more M1 macrophage gene signature sets are:

(a) ACP2, ABCD1, C1QA, FDX1, CCL22, CD163, SCAMP2, ADAMDEC1, ARL8B, and HAMP;

(b) ACP2, ABCD1, FDX1, CCL8, CCL22, CD163, ADAMDEC1, TREM2, and HAMP;

(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB31L1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or

(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

159. The anti-CD20 antibody for use of any one of embodiments 141-154, wherein the macrophage biomarker is a gene expression value.

160. The anti-CD20 antibody for use of embodiment 159, wherein the gene expression value is a median gene expression value.

161. The anti-CD20 antibody for use of embodiment 159 or 160, wherein the gene expression value is measured using a gene signature matrix.

162. The anti-CD20 antibody for use of embodiment 161, wherein the gene signature matrix comprises the following genes:

(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1 PRS, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or

(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

163. The anti-CD20 antibody for use of embodiment 162, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

164. The anti-CD20 antibody for use of any one of embodiments 161-163, wherein the gene signature matrix is used to determine a number of M1 macrophages.

165. The anti-CD20 antibody for use of any one of embodiments 141-164, wherein the macrophage biomarker is an amount of M1 macrophages.

166. The anti-CD20 antibody for use of embodiment 165, wherein the amount of M1 macrophages is measured directly or indirectly.

167. The anti-CD20 antibody for use of embodiment 166, wherein the amount of M1 macrophages is measured directly using flow cytometry, spatial transcriptomics, spatial proteomics, or combination thereof.

168. The anti-CD20 antibody for use of embodiment 166, wherein the amount of M1 macrophages is measured indirectly using nucleic acid or protein.

169. The anti-CD20 antibody for use of embodiment 168, wherein the nucleic acid is measured using RNA-seq, RT-qPCR, qPCR, multiplex qPCR or RT-qPCR, microarray analysis, SAGE, MassARRAY technique, ISH, or a combination thereof.

170. The anti-CD20 antibody for use of embodiment 169, wherein the amount of M1 macrophages is measured using a marker gene approach or a deconvolution approach.

171. The anti-CD20 antibody for use of embodiment 170, wherein the marker gene approach uses xCell.

172. The anti-CD20 antibody for use of embodiment 170, wherein the deconvolution approach uses quanTlseq.

173. The anti-CD20 antibody for use of any one of embodiments 141-154, wherein the macrophage biomarker in the sample from the patient is measured using nucleic acid or protein.

174. The anti-CD20 antibody for use of embodiment 173, wherein the macrophage biomarker in the sample from the patient is determined using a nucleic acid expression level.

175. The anti-CD20 antibody for use of embodiment 174, wherein the nucleic acid expression level is determined by RNA-seq, RT-qPCR, qPCR, multiplex qPCR or RT-qPCR, microarray analysis, SAGE, MassARRAY technique, ISH, or a combination thereof.

176. The anti-CD20 antibody for use of embodiment 174 or 175, wherein the nucleic acid expression level is an mRNA expression level.

177. The anti-CD20 antibody for use of embodiment 176, wherein the mRNA expression level is determined by RNA-seq.

178. The anti-CD20 antibody for use of any one of embodiments 141-177, wherein the sample is a tissue sample, tumor sample, whole blood sample, a plasma sample, a serum sample, or a combination thereof.

179. The anti-CD20 antibody for use of embodiment 178, wherein the sample is a tissue sample.

180. The anti-CD20 antibody for use of embodiment 179, wherein the tissue sample is a tumor tissue sample.

181. The anti-CD20 antibody for use of embodiment 180, wherein the tumor tissue sample contains tumor cells, tumor-infiltrating immune cells, stromal cells, NAT cells, or a combination thereof.

182. The anti-CD20 antibody for use of embodiment 180 or 181, wherein the tumor tissue sample is a biopsy.

183. The anti-CD20 antibody for use of any one of embodiments 178-182, wherein the sample is an archival sample, a fresh sample, or a frozen sample.

184. The anti-CD20 antibody for use of any one of embodiments 141-183, wherein the lymphoma is an indolent lymphoma.

185. The anti-CD20 antibody for use of any one of embodiments 141-184, wherein the lymphoma is a B-cell lymphoma.

186. The anti-CD20 antibody for use of embodiment 184 or 185, wherein the B-cell lymphoma is an NHL.

187. The anti-CD20 antibody for use of any one of embodiments 141-186, wherein the lymphoma is a DLBCL, an FL, a CLL, or an MZL.

188. The anti-CD20 antibody for use of embodiment 187, wherein the lymphoma is a DLBCL.

189. The anti-CD20 antibody for use of embodiment 188, wherein the DLBCL is a GCB or ABC cell-of-origin subgroup of DLBCL.

190. The anti-CD20 antibody for use of any one of embodiments 141-189, wherein the lymphoma is a CD20-positive lymphoma.

191. The anti-CD20 antibody for use of any one of embodiments 141-190, wherein the anti-CD20 antibody is a type I anti-CD20 antibody or a type II anti-CD20 antibody.

192. The anti-CD20 antibody for use of embodiment 191, wherein the anti-CD20 antibody is a type II anti-CD20 antibody.

193. The anti-CD20 antibody for use of embodiment 192, wherein the type II anti-CD20 antibody comprises the following CDRs:

(a) a CDR-H1 with an amino acid sequence of SEQ ID NO: 27;

(b) a CDR-H2 with an amino acid sequence of SEQ ID NO: 28;

(c) a CDR-H3 with an amino acid sequence of SEQ ID NO: 3;

(d) a CDR-L1 with an amino acid sequence of SEQ ID NO: 4;

(e) a CDR-L2 with an amino acid sequence of SEQ ID NO: 5; and

(f) a CDR-L3 with an amino acid sequence of SEQ ID NO: 6.

194. The anti-CD20 antibody for use of embodiment 193, wherein the type II anti-CD20 antibody comprises a VH domain comprising an amino acid sequence of SEQ ID NO: 7 and a VL domain comprising an amino acid sequence of SEQ ID NO: 8.

195. The anti-CD20 antibody for use of embodiment 194, wherein the type II anti-CD20 antibody is obinutuzumab.

196. The anti-CD20 antibody for use of embodiment 191, wherein the anti-CD20 antibody is a type I anti-CD20 antibody.

197. The anti-CD20 antibody for use of embodiment 196, wherein the type I anti-CD20 antibody comprises the following CDRs:

(a) a CDR-H1 with an amino acid sequence of SEQ ID NO: 11;

(b) a CDR-H2 with an amino acid sequence of SEQ ID NO: 12;

(c) a CDR-H3 with an amino acid sequence of SEQ ID NO: 13;

(d) a CDR-L1 with an amino acid sequence of SEQ ID NO: 14;

(e) a CDR-L2 with an amino acid sequence of SEQ ID NO: 15; and

(f) a CDR-L3 with an amino acid sequence of SEQ ID NO: 16.

198. The anti-CD20 antibody for use of embodiment 197, wherein the type I anti-CD20 antibody comprises a VH domain comprising an amino acid sequence of SEQ ID NO: 25 and a VL domain comprising an amino acid sequence of SEQ ID NO: 26.

199. The anti-CD20 antibody for use of embodiment 198, wherein the type I anti-CD20 antibody is rituximab.

200. The anti-CD20 antibody for use of any one of embodiments 141-199, wherein the treatment further comprises use of an effective amount of an additional therapeutic agent.

201. The anti-CD20 antibody for use of embodiment 200, wherein the additional therapeutic agent is one or more of a chemotherapeutic agent, an anti-neoplastic agent, a growth inhibitory agent, an anti-angiogenic agent, a radiation therapy, a cytotoxic agent, or a combination thereof.

202. The anti-CD20 antibody for use of embodiment 201, wherein the additional therapeutic agent is a chemotherapeutic agent.

203. The anti-CD20 antibody for use of embodiment 201 or 202, wherein the chemotherapeutic agent is cyclophosphamide, doxorubicin, vincristine, or prednisone.

204. The anti-CD20 antibody for use of embodiment 201 or 202, wherein the chemotherapeutic agent is cyclophosphamide, doxorubicin, vincristine, and prednisone.

205. The anti-CD20 antibody for use of any one of embodiments 141-204, wherein the patient has not been previously treated for the lymphoma.

206. The anti-CD20 antibody for use of any one of embodiments 141-205, wherein the patient has not been previously administered an anti-CD20 antibody.

207. A method of identifying, diagnosing, and/or predicting whether a patient having a lymphoma may benefit from a treatment comprising an anti-CD20 antibody, the method comprising measuring a Th2 biomarker in a sample from the patient, wherein an amount or level of the Th2 biomarker in the sample that is above a reference Th2 biomarker amount or level identifies, diagnoses, and/or predicts the patient as one who may benefit from a treatment comprising an anti-CD20 antibody.

208. A method of selecting a therapy for a patient having a lymphoma, the method comprising measuring a Th2 biomarker in a sample from the patient, wherein an amount or level of the Th2 biomarker in the sample that is above a reference Th2 biomarker amount or level identifies the patient as one who may benefit from a treatment comprising an anti-CD20 antibody.

209. The method of embodiment 207 or 208, wherein the patient has a Th2 biomarker in the sample that is above a reference Th2 biomarker amount or level, and the method further comprises administering to the patient an effective amount of an anti-CD20 antibody.

210. A method of treating a patient having a lymphoma, the method comprising:

(a) measuring a Th2 biomarker in a sample from the patient, wherein the amount or level of the Th2 biomarker in the sample is above a reference Th2 biomarker amount or level, and

(b) administering an effective amount of an anti-CD20 antibody to the patient based on the Th2 biomarker measured in step (a).

211. A method of treating a patient having a lymphoma, the method comprising administering to the patient an effective amount of an anti-CD20 antibody, wherein prior to treatment the amount or level of a Th2 biomarker in a sample from the patient has been determined to be above a reference Th2 biomarker amount or level.

212. A method of treating a patient having a lymphoma and having an amount or level of a Th2 biomarker in a sample from the patient that is above a reference Th2 biomarker amount or level comprising administering to the patient an effective amount of an anti-CD20 antibody.

213. The method of any one of embodiments 207-212, wherein the reference Th2 biomarker amount or level is a pre-assigned Th2 biomarker amount or level.

214. The method of any one of embodiments 207-213, wherein the reference Th2 biomarker amount or level is an amount or level of a Th2 biomarker in a reference population.

215. The method of embodiment 214, wherein the amount or level of the Th2 biomarker in a reference population is a median amount or level of the Th2 biomarker of the reference population.

216. The method of embodiment 214, wherein the reference Th2 biomarker amount or level is an amount or level of a Th2 biomarker that is at the 25th percentile of the reference population.

217. The method of embodiment 214, wherein the reference Th2 biomarker amount or level is an amount or level of a Th2 biomarker that is at the 50th percentile of the reference population.

218. The method of embodiment 214, wherein the reference Th2 biomarker amount or level is an amount or level of a Th2 biomarker that is at the 75th percentile of the reference population.

219. The method of any one of embodiments 214-218, wherein the reference population is a population of patients having the lymphoma.

220. The method of embodiment 219, wherein the population of patients having the lymphoma was previously treated with an anti-CD20 antibody.

221. The method of embodiment 220, wherein the reference Th2 biomarker amount or level is the amount or level of Th2 biomarker of the reference population prior to initiating treatment with the anti-CD20 antibody.

222. The method of embodiment 220 or 221, wherein the reference Th2 biomarker amount or level significantly separates the reference population into a first set of patients who have benefitted from the treatment with the anti-CD20 antibody and a second set of patients who have not benefitted from the treatment with the anti-CD20 antibody.

223. The method of any one of embodiments 207-222, wherein the reference Th2 biomarker amount or level is an amount of Th2 cells as measured by gene expression.

224. The method of any one of embodiments 207-209, wherein the benefit is an extension of PFS.

225. The method of any one of embodiments 207-209, wherein the benefit is an increase in OS.

226. The method of any one of embodiments 210-212, further comprising achieving an improvement of PFS or OS.

227. The method of any one of embodiments 207-226, wherein the Th2 biomarker is an amount of Th2 cells.

228. The method of embodiment 227, wherein the amount of Th2 cells is measured directly or indirectly.

229. The method of embodiment 228, wherein the amount of Th2 cells is measured directly using flow cytometry, spatial transcriptomics, spatial proteomics, or combination thereof.

230. The method of embodiment 228, wherein the amount of Th2 cells is measured indirectly using nucleic acid or protein.

231. The method of embodiment 230, wherein the nucleic acid is measured using RNA-seq, RT-qPCR, qPCR, multiplex qPCR or RT-qPCR, microarray analysis, SAGE, MassARRAY technique, ISH, or a combination thereof.

232. The method of embodiment 231, wherein the amount of Th2 cells is measured using a marker gene approach or a deconvolution approach.

233. The method of embodiment 232, wherein the marker gene approach uses xCell.

234. The method of embodiment 232, wherein the deconvolution approach uses quanTlseq.

235. The method of any one of embodiments 207-226, wherein the Th2 biomarker in the sample from the patient is measured using nucleic acid or protein.

236. The method of embodiment 235, wherein the Th2 biomarker in the sample from the patient is determined using a nucleic acid expression level.

237. The method of embodiment 236, wherein the nucleic acid expression level is determined by RNA-seq, RT-qPCR, qPCR, multiplex qPCR or RT-qPCR, microarray analysis, SAGE, MassARRAY technique, ISH, or a combination thereof.

238. The method of embodiment 236 or 237, wherein the nucleic acid expression level is an mRNA expression level.

239. The method of embodiment 238, wherein the mRNA expression level is determined by RNA-seq.

240. The method of any one of embodiments 207-239, wherein the sample is a tissue sample, tumor sample, whole blood sample, a plasma sample, a serum sample, or a combination thereof.

241. The method of embodiment 240, wherein the sample is a tissue sample.

242. The method of embodiment 241, wherein the tissue sample is a tumor tissue sample.

243. The method of embodiment 242, wherein the tumor tissue sample contains tumor cells, tumor-infiltrating immune cells, stromal cells, NAT cells, or a combination thereof.

244. The method of embodiment 242 or 243, wherein the tumor tissue sample is a biopsy.

245. The method of any one of embodiments 240-244, wherein the sample is an archival sample, a fresh sample, or a frozen sample.

246. The method of any one of embodiments 207-245, wherein the lymphoma is an indolent lymphoma.

247. The method of any one of embodiments 207-246, wherein the lymphoma is a B-cell lymphoma.

248. The method of embodiment 247, wherein the B-cell lymphoma is a germinal center derived B-cell lymphoma.

249. The method of embodiment 247 or 248, wherein the B-cell lymphoma is an NHL.

250. The method of any one of embodiments 207-249, wherein the lymphoma is a DLBCL, an FL, a CLL, or an MZL.

251. The method of embodiment 250, wherein the lymphoma is a DLBCL.

252. The method of embodiment 251, wherein the DLBCL is a GCB or ABC cell-of-origin subgroup of DLBCL.

253. The method of any one of embodiments 207-252, wherein the lymphoma is a CD20-positive lymphoma.

254. The method of any one of embodiments 207-253, wherein the anti-CD20 antibody is a type I anti-CD20 antibody or a type II anti-CD20 antibody.

255. The method of embodiment 254, wherein the anti-CD20 antibody is a type II anti-CD20 antibody.

256. The method of embodiment 255, wherein the type II anti-CD20 antibody comprises the following CDRs:

(a) a CDR-H1 with an amino acid sequence of SEQ ID NO: 27;

(b) a CDR-H2 with an amino acid sequence of SEQ ID NO: 28;

(c) a CDR-H3 with an amino acid sequence of SEQ ID NO: 3;

(d) a CDR-L1 with an amino acid sequence of SEQ ID NO: 4;

(e) a CDR-L2 with an amino acid sequence of SEQ ID NO: 5; and

(f) a CDR-L3 with an amino acid sequence of SEQ ID NO: 6.

257. The method of embodiment 256, wherein the type II anti-CD20 antibody comprises a VH domain comprising an amino acid sequence of SEQ ID NO: 7 and a VL domain comprising an amino acid sequence of SEQ ID NO: 8.

258. The method of embodiment 257, wherein the type II anti-CD20 antibody is obinutuzumab.

259. The method of embodiment 254, wherein the anti-CD20 antibody is a type I anti-CD20 antibody.

260. The method of embodiment 259, wherein the type I anti-CD20 antibody comprises the following CDRs:

(a) a CDR-H1 with an amino acid sequence of SEQ ID NO: 11;

(b) a CDR-H2 with an amino acid sequence of SEQ ID NO: 12;

(c) a CDR-H3 with an amino acid sequence of SEQ ID NO: 13;

(d) a CDR-L1 with an amino acid sequence of SEQ ID NO: 14;

(e) a CDR-L2 with an amino acid sequence of SEQ ID NO: 15; and

(f) a CDR-L3 with an amino acid sequence of SEQ ID NO: 16.

261. The method of embodiment 260, wherein the type I anti-CD20 antibody comprises a VH domain comprising an amino acid sequence of SEQ ID NO: 25 and a VL domain comprising an amino acid sequence of SEQ ID NO: 26.

262. The method of embodiment 261, wherein the type I anti-CD20 antibody is rituximab.

263. The method of any one of embodiments 209-262, further comprising administering to the patient an effective amount of an additional therapeutic agent.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-4. (canceled)

5. A method of treating a patient having a lymphoma, the method comprising administering to the patient an effective amount of an anti-CD20 antibody, wherein prior to treatment the amount or level of a macrophage biomarker in a sample from the patient has been determined to be above a reference macrophage biomarker amount or level.

6. (canceled)

7. The method of claim 5, wherein the reference macrophage biomarker amount or level is a pre-assigned macrophage biomarker amount or level.

8. The method of claim 5, wherein the reference macrophage biomarker amount or level is:
(a) a median amount or level of the macrophage biomarker in a reference population; or
(b) an amount or level of a macrophage biomarker that is at the 25^{th} percentile, 50^{th} percentile, or 75^{th} percentile of a reference population.

9-12. (cancelled)

13. The method of claim 8, wherein the reference population is a population of patients having the lymphoma.

14. The method of claim 13, wherein the population of patients having the lymphoma was previously treated with an anti-CD20 antibody, and the reference macrophage biomarker amount or level is the amount or level of macrophage biomarker of the reference population prior to initiating treatment with the anti-CD20 antibody.

15. (canceled)

16. The method of claim 14, wherein the reference macrophage biomarker amount or level significantly separates the reference population into a first set of patients who have benefitted from the treatment with the anti-CD20 antibody and a second set of patients who have not benefitted from the treatment with the anti-CD20 antibody.

17-20. (canceled)

21. The method of claim 5, further comprising achieving an improvement of progression-free survival (PFS) or overall survival (OS).

22. The method of claim 5, wherein the macrophage biomarker is an average of M1 macrophage gene signature set scores of one or more M1 macrophage gene signature sets, wherein:
(a) each M1 macrophage gene signature set score is an average of the expression level of one or more genes of an M1 macrophage gene signature set; or
(b) each M1 macrophage gene signature set score is an average of the normalized expression level of one or more genes of an M1 macrophage gene signature set.

23-24. (canceled)

25. The method of claim 22, wherein the one or more M1 macrophage gene signature sets are:
(a) ACP2, ABCD1, C1QA, FDX1, CCL22, CD163, SCAMP2, ADAMDEC1, ARL8B, and HAMP;
(b) ACP2, ABCD1, FDX1, CCL8, CCL22, CD163, ADAMDEC1, TREM2, and HAMP;
(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;
(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB31L1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;
(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB31L1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or
(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

26. The method of claim 5, wherein the macrophage biomarker is a median gene expression value or a gene expression value measured using a gene signature matrix.

27-28. (canceled)

29. The method of claim 26, wherein the gene signature matrix comprises the following genes:
(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or
(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

30. (canceled)

31. The method of claim 26, wherein the gene signature matrix is used to determine a number of M1 macrophages.

32. The method of claim 5, wherein the macrophage biomarker is an amount of M1 macrophages, and wherein:
(a) the amount of M1 macrophages is measured directly using flow cytometry, spatial transcriptomics, spatial proteomics, or combination thereof; or
(b) the amount of M1 macrophages is measured indirectly using nucleic acid or protein.

33-35. (canceled)

36. The method of claim 32, wherein the nucleic acid is measured using RNA-seq, RT-qPCR, qPCR, multiplex qPCR or RT-qPCR, microarray analysis, SAGE, MassARRAY technique, ISH, or a combination thereof.

37. The method of claim 36, wherein the amount of M1 macrophages is measured using a marker gene approach or a deconvolution approach.

38-39. (canceled)

40. The method of claim 5, wherein the macrophage biomarker in the sample from the patient is measured using nucleic acid or protein.

41. The method of claim 40, wherein the macrophage biomarker in the sample from the patient is determined using a nucleic acid expression level, and the nucleic acid expression level is an mRNA expression level.

42-43. (canceled)

44. The method of claim 41, wherein the mRNA expression level is determined by RNA-seq.

45. The method of claim 5, wherein the sample is a tissue sample, tumor sample, whole blood sample, a plasma sample, a serum sample, or a combination thereof.

46-50. (canceled)

51. The method of claim 5, wherein the lymphoma is an indolent lymphoma, a B-cell lymphoma, a diffuse large B-cell lymphoma (DLBCL), a follicular lymphoma (FL), a chronic lymphocytic leukemia (CLL), or a marginal zone lymphoma (MZL).

52. (canceled)

53. The method of claim 51, wherein the B-cell lymphoma is a germinal center derived B-cell lymphoma or a non-Hodgkin lymphoma (NHL).

54-56. (canceled)

57. The method of claim 51, wherein the DLBCL is a germinal-center B-cell-like (GCB) or activated B-cell-like (ABC) cell-of-origin subgroup of DLBCL.

58. The method of claim 5, wherein the lymphoma is a CD20-positive lymphoma.

59. The method of claim 5, wherein the anti-CD20 antibody is a type I anti-CD20 antibody or a type II anti-CD20 antibody.

60. (canceled)

61. The method of claim 59, wherein the anti-CD20 antibody is a type II anti-CD20 antibody comprising the following complementarity determining regions (CDRs):
(a) a CDR-H1 with an amino acid sequence of SEQ ID NO: 27;
(b) a CDR-H2 with an amino acid sequence of SEQ ID NO: 28;
(c) a CDR-H3 with an amino acid sequence of SEQ ID NO: 3;
(d) a CDR-L1 with an amino acid sequence of SEQ ID NO: 4;
(e) a CDR-L2 with an amino acid sequence of SEQ ID NO: 5; and
(f) a CDR-L3 with an amino acid sequence of SEQ ID NO: 6.

62. The method of claim 61, wherein the type II anti-CD20 antibody comprises a VH domain comprising an amino acid sequence of SEQ ID NO: 7 and a VL domain comprising an amino acid sequence of SEQ ID NO: 8.

63. The method of claim 62, wherein the type II anti-CD20 antibody is obinutuzumab.

64. (canceled)

65. The method of claim 59, wherein the anti-CD20 antibody is a type I anti-CD20 antibody comprising the following CDRs:
(a) a CDR-H1 with an amino acid sequence of SEQ ID NO: 11;
(b) a CDR-H2 with an amino acid sequence of SEQ ID NO: 12;
(c) a CDR-H3 with an amino acid sequence of SEQ ID NO: 13;
(d) a CDR-L1 with an amino acid sequence of SEQ ID NO: 14;
(e) a CDR-L2 with an amino acid sequence of SEQ ID NO: 15; and
(f) a CDR-L3 with an amino acid sequence of SEQ ID NO: 16.

66. The method of claim 65, wherein the type I anti-CD20 antibody comprises a VH domain comprising an amino acid sequence of SEQ ID NO: 25 and a VL domain comprising an amino acid sequence of SEQ ID NO: 26.

67. The method of claim 66, wherein the type I anti-CD20 antibody is rituximab.

68. The method of claim 5, further comprising administering to the patient an effective amount of an additional therapeutic agent.

69. The method of claim 68, wherein the additional therapeutic agent is one or more of a chemotherapeutic agent, an anti-neoplastic agent, a growth inhibitory agent, an anti-angiogenic agent, a radiation therapy, a cytotoxic agent, or a combination thereof.

70. (canceled)

71. The method of claim 69, wherein:
(a) the chemotherapeutic agent is cyclophosphamide, doxorubicin, vincristine, or prednisone; or
(b) the chemotherapeutic agent is cyclophosphamide, doxorubicin, vincristine, and prednisone.

72. (canceled)

73. The method of claim 5, wherein the patient has not been previously treated for the lymphoma.

74. The method of claim 5, wherein the patient has not been previously administered an anti-CD20 antibody.

75-210. (canceled)

211. A method of treating a patient having a lymphoma, the method comprising administering to the patient an effective amount of an anti-CD20 antibody, wherein prior to treatment the amount or level of a Th2 biomarker in a sample from the patient has been determined to be above a reference Th2 biomarker amount or level.

212-263. (canceled)


SEQUENCE LISTING (28 sequences)

SEQ ID NO: 1 - protein, 6 aa, synthetic construct
GYAFSY

SEQ ID NO: 2 - protein, 8 aa, synthetic construct
FPGDGDTD

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 115 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 9 - protein, 448 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYNMH

SEQ ID NO: 12 - protein, 17 aa, synthetic construct
AIYPGNGDTSYNQKFKG

SEQ ID NO: 13 - protein, 12 aa, synthetic construct
STYYGGDWYFNV

SEQ ID NO: 14 - protein, 10 aa, synthetic construct
RASSSVSYIH

SEQ ID NO: 15 - protein, 7 aa, synthetic construct
ATSNLAS

SEQ ID NO: 16 - protein, 9 aa, synthetic construct
QQWTSNPPT

SEQ ID NO: 17 - protein, 23 aa, synthetic construct
QIVLSQSPAILSASPGEKVTMTC

SEQ ID NO: 18 - protein, 15 aa, synthetic construct
WFQQKPGSSPKPWIY

SEQ ID NO: 19 - protein, 32 aa, synthetic construct
GVPVRFSGSGSGTSYSLTISRVEAEDAATYYC

SEQ ID NO: 20 - protein, 10 aa, synthetic construct
FGGGTKLEIK

SEQ ID NO: 21 - protein, 30 aa, synthetic construct
QVQLQQPGAELVKPGASVKMSCKASGYTFT

SEQ ID NO: 22 - protein, 14 aa, synthetic construct
WVKQTPGRGLEWIG

SEQ ID NO: 23 - protein, 32 aa, synthetic construct
KATLTADKSSSTAYMQLSSLTSEDSAVYYCAR

SEQ ID NO: 24 - protein, 11 aa, synthetic construct
WGAGTTVTVSA

SEQ ID NO: 25 - protein, 121 aa, synthetic construct [sequence omitted]

SEQ ID NO: 26 - protein, 106 aa, synthetic construct [sequence omitted]

SEQ ID NO: 27 - protein, 5 aa, synthetic construct
YSWIN

SEQ ID NO: 28 - protein, 16 aa, synthetic construct
RIFPGDGDTDYNGKFK